SEQUENCE LISTING (56 sequences)

SEQ ID NO: 1 - DNA, 4107 bp, Streptococcus pyogenes
  CDS (1)..(4107)
atggataagaaatactcaataggcttagatatcggcacaaatagcgtcggatgggcggtgatcactgatgattataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 2 - protein, 1368 aa, Streptococcus pyogenes
MDKKYSIGLDIGTNSVGWAVITDDYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 3 - DNA, 2211 bp, Artificial Sequence
  AAV Capsid Ortholog sequence
  CDS (1)..(2211)
atgtcttttgttgaccaccctccagattggttggaatcgatcggcgacggctttcgtgaatttctcggccttgaggcgggtcccccgaaacccaaggccaatcaacagaagcaagataacgctcgaggtcttgtgcttcctgggtacaagtatcttggtcctgggaacggccttgataagggcgatcctgtcaattttgctgacgaggttgcccgagagcacgacctctcctaccagaaacagcttgaggcgggcgataacccttacctcaagtacaaccacgcggacgcagagtttcaggagaaactcgcttctgacacttcttttggaggaaaccttgggaaggctgttttccaggctaaaaagaggcttctcgaacctcttggcctggttgagacgccggataaaacggcgcctgcggcaaaaaagaggcctctagagcagagtcctcaagagccagactcctcgagcggatttggcaaaaagggcaaacagcctgccagaaagagactcaattttgacgacgaacctggagccggagacgggcctcccccagaaggaccatcttccggagctatgtctactgagactgaaatgcgtgcagcagctggcggaaatggtggcgatgcgggacaaggtgccgagggagtgggtaatgcctccggtgattggcattgcgattccacttggtcagagagccacgtcaccaccacctcaacccgcacctgggtcctgccgacctacaacaaccacctgtacctgcggctcggcacgagcaacgccagcgacaccttcaacggattctccaccccctggggatactttgacttcaaccgcttccactgccacttctcgccaagagactggcaaaggctcatcaacaaccactgggggctgcgccccaaaagcatgcaagtccgcatcttcaacatccaagttaaggaggtcacgacgtctaacggagagacgaccgtatcaaacaacctcaccagcacggtccagatctttgcggacagcacgtacgagctcccgtacgtgatggatgcgggtcaggagggcagcttgccccctttccccaacgacgtgttcatggtgccccagtacgggtactgcggactggtaaccggaggcagctctcaaaaccagacagacagaaacgccttctactgtctggagtactttcccagccagatgctgagaacaggaaacaactttgagatggtgtacaagtttgagaacgtgcccttccactccatgtacgctcacagccagagcctggataggctgatgaacccgctgctggaccagtacctgtgggaactccagtctaccacatctggaggaaatctcaaccagggcacgtcagccacaaactttgccaagctgaccaaaacaaacttttctggctaccgcaaaaactggcttccgggacccatgatgaagcagcagagattctccaagactgccaatcaaaactacaagattccccaagggggaaataacagtctgctccattatgagaccagaactaccctcgacggaagatggagcaattttgccccgggaacggccatggcaaccgcggccaacgatgccaccgacttctcccaggcccagctcatctttgcggggcccaacatcaccggcaataccacgacagacgccaacaacctgatgttcacttcagaagatgaactcagggccaccaacccccgggacactgacctgtttggccacctggcaaccaaccagcaaaacgccaccaccgttcctaccgcagacgacgtggacggagtcggcgtgtacccgggaatggtgtggcaggacagagacatttattaccaagggcccatctgggccaagattccccatacggatggacactttcacccatctcccctcattggcggatttggactgaaaagcccgcctccacaaatattcatcaaaaacacccctgtacccgccaatcccgcgacgaccttctctccggccagaatcaacagcttcatcacccagtacagcaccggacaggtggctgtcaaaatagaatgggaaatccagaaggagcggtccaagagatggaacccagaggtccagttcacgtccaactacggagcacaggactcgcttctctgggctcccgacaacgccggagcctacaaagagcccagggccattggatcccgatacctcaccaaccacctctag

SEQ ID NO: 4 - protein, 736 aa, Artificial Sequence
  Synthetic Construct
MSFVDHPPDWLESIGDGFREFLGLEAGPPKPKANQQKQDNARGLVLPGYKYLGPGNGLDKGDPVNFADEVAREHDLSYQKQLEAGDNPYLKYNHADAEFQEKLASDTSFGGNLGKAVFQAKKRLLEPLGLVETPDKTAPAAKKRPLEQSPQEPDSSSGFGKKGKQPARKRLNFDDEPGAGDGPPPEGPSSGAMSTETEMRAAAGGNGGDAGQGAEGVGNASGDWHCDSTWSESHVTTTSTRTWVLPTYNNHLYLRLGTSNASDTFNGFSTPWGYFDFNRFHCHFSPRDWQRLINNHWGLRPKSMQVRIFNIQVKEVTTSNGETTVSNNLTSTVQIFADSTYELPYVMDAGQEGSLPPFPNDVFMVPQYGYCGLVTGGSSQNQTDRNAFYCLEYFPSQMLRTGNNFEMVYKFENVPFHSMYAHSQSLDRLMNPLLDQYLWELQSTTSGGNLNQGTSATNFAKLTKTNFSGYRKNWLPGPMMKQQRFSKTANQNYKIPQGGNNSLLHYETRTTLDGRWSNFAPGTAMATAANDATDFSQAQLIFAGPNITGNTTTDANNLMFTSEDELRATNPRDTDLFGHLATNQQNATTVPTADDVDGVGVYPGMVWQDRDIYYQGPIWAKIPHTDGHFHPSPLIGGFGLKSPPPQIFIKNTPVPANPATTFSPARINSFITQYSTGQVAVKIEWEIQKERSKRWNPEVQFTSNYGAQDSLLWAPDNAGAYKEPRAIGSRYLTNHL

SEQ ID NO: 5 - DNA, 2211 bp, Artificial Sequence
  AAV Capsid Ortholog sequence
  CDS (1)..(2211)
atgtcttttgttgaccaccctccagattggttggaatcgatcggcgacggctttcgtgaatttctcggccttgaggcgggtcccccgaaacccaaggccaatcaacagaagcaagataacgctcgaggtcttgtgcttcctgggtacaagtatcttggtcctgggaacggccttgataagggcgatcctgtcaattttgctgacgaggttgcccgagagcacgacctctcctaccagaaacagcttgaggcgggcgataacccttacctcaagtacaaccacgcggacgcagagtttcaggagaaactcgcttctgacacttcttttgggggaaaccttgggaaggctgttttccaggctaaaaagaggattctcgaacctcttggcctggttgagacgccggataaaacggcgcctgcggcaaaaaagaggcctctagagcagagtcctcaagagccagactcctcgagcggagttggcaagaaaggcaaacagcctgccagaaagagactcaactttgacgacgaacctggagccggagacgggcctcccccagaaggaccatcttccggagctatgtctactgagactgaaatgcgtgcagcagctggcggaaatggtggcgatgcgggacaaggtgccgagggagtgggtaatgcctccggtgattggcattgcgattccacttggtcagagagccacgtcaccaccacctcaacccgcacctgggtcctgccgacctacaacaaccacctgtacctgcggctcggctcgagcaacgccagcgacaccttcaacggattctccaccccctggggatactttgactttaaccgcttccactgccacttctcgccaagagactggcaaaggctcatcaacaaccactggggactgcgccccaaaagcatgcaagtccgcatcttcaacatccaagttaaggaggtcacgacgtctaacggggagacgaccgtatccaacaacctcaccagcacggtccagatctttgcggacagcacgtacgagctcccgtacgtgatggatgcaggtcaggagggcagcttgcctcctttccccaacgacgtgttcatggtgcctcagtacgggtactgcggactggtaaccggaggcagctctcaaaaccagacagacagaaatgccttctactgtctggagtactttcccagccagatgctgagaaccggaaacaactttgagatggtgtacaagtttgaaaacgtgcccttccactccatgtacgctcacagccagagcctggataggctgatgaacccgctgctggaccagtacctgtgggagctccagtctaccacctctggaggaactctcaaccagggcaattcagccaccaactttgccaagctgaccaaaacaaacttttctggctaccgcaaaaactggctcccggggcccatgatgaagcagcagagattctccaagactgccagtcaaaactacaagattccccagggaagaaacaacagtctgctccattatgagaccagaactaccctcgacggaagatggagcaattttgccccgggaacggccatggcaaccgcagccaacgacgccaccgacttctctcaggcccagctcatctttgcggggcccaacatcaccggcaacaccaccacagatgccaataacctgatgttcacttcagaagatgaacttagggccaccaacccccgggacactgacctgtttggccacctggcaaccaaccagcaaaacgccaccaccgttcctaccgtagacgacgtggacggagtcggcgtgtacccgggaatggtgtggcaggacagagacatttactaccaagggcccatttgggccaaaattccacacacggatggacactttcacccgtctcctctcattggcggatttggactgaaaagcccgcctccacaaatattcatcaaaaacactcctgtacccgccaatcccgcaacgaccttctctccggccagaatcaacagcttcatcacccagtacagcaccggacaggtggctgtcaaaatagaatgggaaatccagaaggagcggtccaagagatggaacccagaggtccagttcacgtccaactacggagcacaggactcgcttctctgggctcccgacaacgccggagcctacaaagagcccagggccattggatcccgatacctcaccaaccacctctag

SEQ ID NO: 6 - protein, 736 aa, Artificial Sequence
  Synthetic Construct
MSFVDHPPDWLESIGDGFREFLGLEAGPPKPKANQQKQDNARGLVLPGYKYLGPGNGLDKGDPVNFADEVAREHDLSYQKQLEAGDNPYLKYNHADAEFQEKLASDTSFGGNLGKAVFQAKKRILEPLGLVETPDKTAPAAKKRPLEQSPQEPDSSSGVGKKGKQPARKRLNFDDEPGAGDGPPPEGPSSGAMSTETEMRAAAGGNGGDAGQGAEGVGNASGDWHCDSTWSESHVTTTSTRTWVLPTYNNHLYLRLGSSNASDTFNGFSTPWGYFDFNRFHCHFSPRDWQRLINNHWGLRPKSMQVRIFNIQVKEVTTSNGETTVSNNLTSTVQIFADSTYELPYVMDAGQEGSLPPFPNDVFMVPQYGYCGLVTGGSSQNQTDRNAFYCLEYFPSQMLRTGNNFEMVYKFENVPFHSMYAHSQSLDRLMNPLLDQYLWELQSTTSGGTLNQGNSATNFAKLTKTNFSGYRKNWLPGPMMKQQRFSKTASQNYKIPQGRNNSLLHYETRTTLDGRWSNFAPGTAMATAANDATDFSQAQLIFAGPNITGNTTTDANNLMFTSEDELRATNPRDTDLFGHLATNQQNATTVPTVDDVDGVGVYPGMVWQDRDIYYQGPIWAKIPHTDGHFHPSPLIGGFGLKSPPPQIFIKNTPVPANPATTFSPARINSFITQYSTGQVAVKIEWEIQKERSKRWNPEVQFTSNYGAQDSLLWAPDNAGAYKEPRAIGSRYLTNHL

SEQ ID NO: 7 - DNA, 2151 bp, Artificial Sequence
  AAV Capsid Ortholog sequence
  CDS (1)..(2151)
atgtcgttctttgattatctaggtaaaaaatacgctcaaggagccgcggattggtgggacgcgaaagcaggaccacctcctccgaaaaaggccaaagggaaaggcttcgtctttcccggacacaactacctaggtcccggcaacgacctagacaacggtaaacccgttgacgcggacgacgctgccgccaaaaagcacgacgaagcttacgacgagcagctcaaacagggcgacaacccgtacgtgaaatataatcacgcggacgcccagctgcaagaagacctcaaagacgtcaatacctttgaggccaatctcgccaaaggagtcttccaggccaagaaacgggtcctcgaaccgtttggtctggtagaagaactggccgcaaagaagcgacctcactctgagatcgaatcgcccgattccagctcgggaatcggcaagaagggagaccaacccgcacgacgtcggttggactttgaccaggaagacagacaacaagccgctcaagacggagccaccggtaccggaagtctcgagccgtcctcaaatttgggatctgatacgatgtctacaggcggtggcgctccaatggcaggagataaccaaggcgccgatggagtgggtaatgcctcaggaaattggcattgcgataccacatggatgggagaccgagtggtcacccgctccactcgcacctggttgctgcccagctacaacaaccacgtgtacagaagaatcaacgccggatcaaacagcaacgccttcttcggcttcagcaccccgtgggggtactttgactacaacagattccactgccacttttctccacgagactggcaacgactcgtcaacaacaactggggcattcgacccaagcacctcaagttcaaactcttcaacatacaagttaaagaatacacggtccaggagggcggaggcgagaccgtcgccaataacctcacaagcaccgtgcaggtctttacggactcggactatcagctaccgtacgtgctaggttcggcgcaagagggtgccatgccaccgttccccgccgacgtgtttatgttgcctcaatacgcgtactgcactcggcagaacggaaccaacccgacgcccagaagcgccttttactgtctggaatactttcccagtcagatgctgagaaccggcaacaactttgacttctcgtacgagttcgagaacgtgcccttccacagtatgtgggctcacaaccagactcttgatcgtctgatgaatcctctggtggatcaatacttgtggagactacaaaatgtagctaacgtaagcggaacaaagaccaatcagttcagccagaacgtggccggcgacctacaactgtttactcggaactggctacccgggcccgcttacagaacgcagtcttggtcgaccaacacggctcagaacgcgactcagaccaacatcccgaatcaggaaaacgtctactttctggaaggaagaactaacaccatcgcacccggcatggccatggggcccacaaccgctacacactttcaatccaacggagtgatattcgccaagaacgaatcgggtactccggcaactggtgccaacattaaccagttgaacatcacacgggaatcagaagtcaactcgaccaatcctctgctgtcaagaccacccctggctattaccaacaacgagcagaccgcagctgccacgccagacagagccaactggaatctggctcccgtcatgcccggacaagtctggatggacaaagacatctacctgcaaggccagatctgggccaagatcccacacacggacggacactttcatccgtctcctctcatgggaggattcggactgaaacaccctccacctcaaatcttcatcaagaacactcccgtacccggatcacccgctcccaactttaccgtggacaaggtcaacaagttcatcaaccaatactctaccggtcaagtaaccgtggagattgaatgggaactgcagaaggaatcgtccaagagatggaatccagagatccaatacacggacgacgcatccgccaccgacggatctatcctgcactttgctcccaacgacgaaggattgtacacccagttccgatctatcgggactcggtacctcacccgccctctgtaa

SEQ ID NO: 8 - protein, 716 aa, Artificial Sequence
  Synthetic Construct
MSFFDYLGKKYAQGAADWWDAKAGPPPPKKAKGKGFVFPGHNYLGPGNDLDNGKPVDADDAAAKKHDEAYDEQLKQGDNPYVKYNHADAQLQEDLKDVNTFEANLAKGVFQAKKRVLEPFGLVEELAAKKRPHSEIESPDSSSGIGKKGDQPARRRLDFDQEDRQQAAQDGATGTGSLEPSSNLGSDTMSTGGGAPMAGDNQGADGVGNASGNWHCDTTWMGDRVVTRSTRTWLLPSYNNHVYRRINAGSNSNAFFGFSTPWGYFDYNRFHCHFSPRDWQRLVNNNWGIRPKHLKFKLFNIQVKEYTVQEGGGETVANNLTSTVQVFTDSDYQLPYVLGSAQEGAMPPFPADVFMLPQYAYCTRQNGTNPTPRSAFYCLEYFPSQMLRTGNNFDFSYEFENVPFHSMWAHNQTLDRLMNPLVDQYLWRLQNVANVSGTKTNQFSQNVAGDLQLFTRNWLPGPAYRTQSWSTNTAQNATQTNIPNQENVYFLEGRTNTIAPGMAMGPTTATHFQSNGVIFAKNESGTPATGANINQLNITRESEVNSTNPLLSRPPLAITNNEQTAAATPDRANWNLAPVMPGQVWMDKDIYLQGQIWAKIPHTDGHFHPSPLMGGFGLKHPPPQIFIKNTPVPGSPAPNFTVDKVNKFINQYSTGQVTVEIEWELQKESSKRWNPEIQYTDDASATDGSILHFAPNDEGLYTQFRSIGTRYLTRPL

SEQ ID NO: 9 - DNA, 2157 bp, Artificial Sequence
  AAV Capsid Ortholog sequence
  CDS (1)..(2157)
atgtcttctttgtttaaagagtacctgcagagtaccggactggtgggtatccagtcgggagcgccgaagccaaaggccggccagcagaagcaggatacagggtcctttgagtggaaaaagaaggaggacacagccagagggctggtccttcccggttacaagtacctcgggcccttcaacggtctagagcggggcgagcctgtgaatgccgcggacgccgcggcccagcgacacgaccgacagtacgatcgtattctacaacaagggggtaatccatacctcacgtacaaccacgccgaccgagagttccaggaggagctccagtctgacgagtcttttgggggaaatcttggcaaggctgtgtttcaggccaagaagcgaatcctagagcccctcggtctggttgaagaggatccctctcagccagattcgtcctcggacaacacggcgccacccgtgaaaaagtcccgtctcgaagaagcccagcccattcaaagtccagacgtttccagcagcactggcggaggaattgccgacgtcatgtctggagatgctgaaatggctgcagtgggcgggggagcaccgggcgtcgacggccagggtgccgagggagtgggtacttcctcgggtaattggcattgcgattcccagtggtcagaaggacacgtcagaaccaccagcaccagaacctgggtgttgcccagctacaacaaccacctgtataaacggcttggaagtagcgcacaatccaatacctacaacggattctccaccccctggggatacctcgacttcaatagatggcactgtcacttcagtcctcggaactggcaacgtctcatcaacaacaactggggcatcagaccaaaaagacttaatgttaaattgttcaacatacaagtcaaagaggtcacgacggaaggggggacgacgaccgtcgccaataaccttaccagcacgattcaggtgtttgcggacaacgcgtacgaactcccgtatgttgtcgacgcgggtcacgagggggcattgccgccgttcccaaacgacgtgtttatgattccccaatacggatactgcgggctggtgtctggtcaaagtcaggctcagtcggacttgtgttcgttttattgcctggagtattttccatcccagatgctgagaacaggaaacaattttgaaatgaatttcaggtttgaagacgttccatttcactccatgtacgcccacagccagtctctagacaggctcatgaatcctttaattgatcagtatctgtggagtctaaaacaaacaggaaacccgactggatcaacaacgagagacttaaaatttatcaagaacaaagttcccaattttgcacattatggaaaaaattggcttcctggaccttttattagacaacaggggtggacaacacaaaatattaataatagtgttgttaattttaatgacatgctgggaaaaaattcgacatttactttggacactagatggagttcattagcgcctggtccgtgtatgggggatgacggacgaactccatccaccaccaagttctcaaatgctcagctcatgtttggatctggaacacaacccaccgaaggcggtgaagatgctgtacatattacatccgagtcggaggtcaaggcaaccaacccaactgcaatcgatgaatacggacgagtggccgataatacgcaaaatgcaacaaccgccccaaccacagtgggaaatgctgcaatgggggccatgcctgggatggtgtggcaagatagggatatctatcttcaaggacccatctggggaaaaataccccatacagacggacattttcatccgtctcctctcatgggggggtttggatacagaaaaccccctccgcaaatttttattaaaaacacccccgttcctggaaatccggcaactacattttctccaaatagaataaacaatttcattactcaatactcaacgggacaagtgaccgttactattgactgggagctgcaaaaggaaaactcaaagagatggaacccagaagtacaatttacatcaaattttggcacggtcgattcactaaactgggcaccggacaacgcgggaaactacaaagaaccaagggtggtggggtctcgattccttacgcatatactataa

SEQ ID NO: 10 - protein, 718 aa, Artificial Sequence
  Synthetic Construct
MSSLFKEYLQSTGLVGIQSGAPKPKAGQQKQDTGSFEWKKKEDTARGLVLPGYKYLGPFNGLERGEPVNAADAAAQRHDRQYDRILQQGGNPYLTYNHADREFQEELQSDESFGGNLGKAVFQAKKRILEPLGLVEEDPSQPDSSSDNTAPPVKKSRLEEAQPIQSPDVSSSTGGGIADVMSGDAEMAAVGGGAPGVDGQGAEGVGTSSGNWHCDSQWSEGHVRTTSTRTWVLPSYNNHLYKRLGSSAQSNTYNGFSTPWGYLDFNRWHCHFSPRNWQRLINNNWGIRPKRLNVKLFNIQVKEVTTEGGTTTVANNLTSTIQVFADNAYELPYVVDAGHEGALPPFPNDVFMIPQYGYCGLVSGQSQAQSDLCSFYCLEYFPSQMLRTGNNFEMNFRFEDVPFHSMYAHSQSLDRLMNPLIDQYLWSLKQTGNPTGSTTRDLKFIKNKVPNFAHYGKNWLPGPFIRQQGWTTQNINNSVVNFNDMLGKNSTFTLDTRWSSLAPGPCMGDDGRTPSTTKFSNAQLMFGSGTQPTEGGEDAVHITSESEVKATNPTAIDEYGRVADNTQNATTAPTTVGNAAMGAMPGMVWQDRDIYLQGPIWGKIPHTDGHFHPSPLMGGFGYRKPPPQIFIKNTPVPGNPATTFSPNRINNFITQYSTGQVTVTIDWELQKENSKRWNPEVQFTSNFGTVDSLNWAPDNAGNYKEPRVVGSRFLTHIL

SEQ ID NO: 11 - DNA, 2208 bp, Artificial Sequence
  AAV Capsid Ortholog sequence
  CDS (1)..(2208)
atgtcttttttagaagcgtttgaggactggtacgaaaaagcagcggctacttggcgacatctcgaacctggcgccccccgtcctaaagccaaccagcagcatcaggaacagcatcatccggttcctgctgaagagcctaaagatcaagctcgaggccttgtgctacctggctacaagtacctcggcccctttaacggccttgataaaggtgatccggttaatcaagcggacgctgcggcgcttgaacacgataaagcgtacaaccaccttctcgaagaaggacaaaacccgtacctcgtgtataacgaggctgataagcatttccaggaaactctacagggggatacgagcttcggcggtaacctcggcaaagcagtcttccaaggcaagaaacgagtcctagagccctttggactcgttgacgaggagcctcacgaactggctccacctccaaaaaagaagacaaaacaggaatacaaagacactctagaggctattcctcctcctagcagagaggaacagactcctcagaagggatcttcagcatccaaaaacggagcctcatcttcagcagcagcagcagcccccagtaatttgggatctggtatcatggcagaaggcggtggcgcaccaatgggcgacaatcaacagggtgccgatggagtgggtaatgcctcgggaaattggcattgcgattcccactggatgggcgacaaagtcgtcacccgcaccaccagaacctgggtcctgcccacctacaacaaccacctctaccaagccatcgacgagaatactactctcggatccgccaacaacttctacggattcagcaccccatgggggtactttgactttaaccgcttccactgccacttctccccacgtgactggcaaagactcatcaacaacaactggggcatccgacccagaagcctcagattcaagctgttcaacatccaagtcaaggaggtcacgacgacgtcgggcgaaacgaccatcgccaataaccttaccagcacggttcaggtttttgcggatgaagaaatgcacctcccgtacgtcttagggagtgcccacgacgggtgctttcctccatttccaccagacatttacatgctgccccaatacgggtactgcactctggactatcagggactcaccacagacaggagcgccttctactgtctggagtacttcccttctcagatgctgagaacgggcaacaactttgagcttacttacaactttgagaagctgcctttccacagcatgtggatgcacaaccagtccctggacaggatcatgaatcctctcatcgatcagtacctgtatcgcttcgcctccaagactggcaacacatggacataccaaaaaggcagcaaagacgacaagaccgctcaagccagaaactggcttcctggaccctccatcagaaatcagcctctgtactcgagtggtaatcaaaccaacctctcgggatacgatatcgctccaaaaacagaagtcaacgagaaacagatgactgtgtttcctggactttctatggccaccgaaaacgagaggagcggaaccatcaaagagcagccaacgtccaatctgctcatctttgccaacaaagcggatgcggctacctctcagaccacttctagcgtgggcagtacagcgacactcctggtcaccaacgaaaacgaagtcaagactgtcaatccctccgccaccgaagcgtggggttcggctgcggccaacaaacagacatctgctaccgcctctcaaagcgtggctattcacgctcaaggcgtggtgccaggcatggtctggcaagacagagacatctactttcagggccccatctgggccaagatcccgcatacagatggacactttcacccatctcctctcatgggtggattcggactcaaacatcctcctccacaaattttcattaaaaacactcctgtcccctccaatcctgctaccacgtacaccaccgccaagtacaactccttcatcacccagtactccaccggtcaagtgactgtggagattgactgggaactccagaaggagacctccaaaaagtggaaccccgagatccagttcacctccaattacaacgggacagacacgctcggattcgctccagatgccgctggagactacaccgaaccacgtgccatcggaacccgataccttacccgacccctttaa

SEQ ID NO: 12 - protein, 735 aa, Artificial Sequence
  Synthetic Construct
MSFLEAFEDWYEKAAATWRHLEPGAPRPKANQQHQEQHHPVPAEEPKDQARGLVLPGYKYLGPFNGLDKGDPVNQADAAALEHDKAYNHLLEEGQNPYLVYNEADKHFQETLQGDTSFGGNLGKAVFQGKKRVLEPFGLVDEEPHELAPPPKKKTKQEYKDTLEAIPPPSREEQTPQKGSSASKNGASSSAAAAAPSNLGSGIMAEGGGAPMGDNQQGADGVGNASGNWHCDSHWMGDKVVTRTTRTWVLPTYNNHLYQAIDENTTLGSANNFYGFSTPWGYFDFNRFHCHFSPRDWQRLINNNWGIRPRSLRFKLFNIQVKEVTTTSGETTIANNLTSTVQVFADEEMHLPYVLGSAHDGCFPPFPPDIYMLPQYGYCTLDYQGLTTDRSAFYCLEYFPSQMLRTGNNFELTYNFEKLPFHSMWMHNQSLDRIMNPLIDQYLYRFASKTGNTWTYQKGSKDDKTAQARNWLPGPSIRNQPLYSSGNQTNLSGYDIAPKTEVNEKQMTVFPGLSMATENERSGTIKEQPTSNLLIFANKADAATSQTTSSVGSTATLLVTNENEVKTVNPSATEAWGSAAANKQTSATASQSVAIHAQGVVPGMVWQDRDIYFQGPIWAKIPHTDGHFHPSPLMGGFGLKHPPPQIFIKNTPVPSNPATTYTTAKYNSFITQYSTGQVTVEIDWELQKETSKKWNPEIQFTSNYNGTDTLGFAPDAAGDYTEPRAIGTRYLTRPL

SEQ ID NO: 13 - DNA, 2181 bp, Artificial Sequence
  AAV Capsid Ortholog sequence
  CDS (1)..(2181)
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcacaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgcacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactattggggcttcagaccccggtctctcagagtcaaaatcttcaacatccaagtcaaagaggtcacggtgcaggactccaccaccaccatcgccaacaacctcacctccaccgtccaagtgtttacggacgacgactaccaactcccgtacgtcgtcggcaacgggaccgagggatgcctgccggccttccccccgcaggtctttacgctgccgcagtacggctacgcgacgctgaaccgagacaacggagacaacccgacagagcggagcagcttcttttgcctagagtactttcccagcaagatgctgaggacgggcaacaactttgagtttacctacagctttgaagaggtgcccttccactgcagcttcgccccgagccagaacctctttaagctggccaacccgctggtggaccagtacctgtaccgcttcgtgagcacctcggccacgggcgccatccagttccaaaagaacctggcgggcagatacgccaacacctacaaaaactggttcccggggcccatgggccgaacccagggctggaacacgagctctggcagcagcaccaacagagtcagcgtcaacaacttttccgtctcaaaccggatgaacctggagggggccagctaccaagtgaacccccagcccaacgggatgacaaacacgctccaaggcagcaaccgctacgcgctggaaaacaccatgatcttcaacgctcaaaacgccacgccgggaactacctcggtgtacccagaggacaatctactgctgaccagcgagagcgagactcagcccgtcaaccgggtggcttacaacacgggcggtcagatggccaccaacgcccagaacgccaccacggctcccacggtcgggacctacaacctccaggaagtgcttcctggcagcgtatggatggagagggacgtgtacctccaaggacccatctgggccaagatcccagagacgggggcgcactttcacccctctccggccatgggcggattcggactcaaacacccgccgcccatgatgctcatcaaaaacacgccggtgcccggcaacatcaccagcttctcggacgtgcccgtcagcagcttcatcacccagtacagcaccgggcaggtcaccgtggagatggaatgggagctcaaaaaggaaaactccaagaggtggaacccagagatccagtacaccaacaactacaacgacccccagtttgtggactttgctccagacggctccggcgaatacagaaccaccagagccatcggaacccgatacctcacccgacccctttaa

SEQ ID NO: 14 - protein, 726 aa, Artificial Sequence
  Synthetic Construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNGDNPTERSSFFCLEYFPSKMLRTGNNFEFTYSFEEVPFHCSFAPSQNLFKLANPLVDQYLYRFVSTSATGAIQFQKNLAGRYANTYKNWFPGPMGRTQGWNTSSGSSTNRVSVNNFSVSNRMNLEGASYQVNPQPNGMTNTLQGSNRYALENTMIFNAQNATPGTTSVYPEDNLLLTSESETQPVNRVAYNTGGQMATNAQNATTAPTVGTYNLQEVLPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDGSGEYRTTRAIGTRYLTRPL

SEQ ID NO: 15 - DNA, 2151 bp, Artificial Sequence
  AAV Capsid Ortholog sequence
  CDS (1)..(2151)
atgtcgtttgttgatcaccctccagattggcttgaggagattggtgagggtctaaaggagtttttgggactcgaacctggcccacccaaaccgaagcccaaccagcagaagcaagacgacgcccgtggtcttgtactgcctggatataattacctgggacccggaaacggtctcgaccgcggagaacctgtcaaccgggctgacgaggtcgcgcgagagcacgacatctcgtacaacgagcagctccaggcgggagacaacccctacctcaagtacaaccacgcggacgccgagtttcaggagaagctcgcggacgacacctccttcgggggcaacctcggcaaggcagtctttcaggccaaaaaaagggttctcgaaccttttggcctggttgaggagcctgttaagacggctgctaaaggcgagcggatagacgaccactatcccaaaaagaagaaggctcggatcgaagagaccgaagctggaaccagcggagcccagcagctgcagatcccagcccaaccagcctcaagtttgggagctgatacaatgtctgcgggaggtggcagcccactgggcgacaataaccaaggcgccgatggagtgggcaatgcctcgggagattggcattgcgattccacgtggatgggggaccgagtcatcaccaagtccacccgaacctgggtgctgcccagctacaacaaccatcagtaccttgagatccacagcggttccgtcgacggaagcaacgctaacgcttattttggatacagcaccccctgggggtactttgacttcaaccgcttccacagccactggagcccccgagactggcagcgactcgtcaacaactactggggattcagaccccggtccctcaaggtcaagatctttaacatccaagtcaaggaagtcacgacgcaggacggcaccaccaccatcgccaacaacctcacctccaccgtccaagtgtttacggacaacgactaccagctaccgtacgtcatcggcaacggaacggaggggtgcctgccggccttccctccgcaggtctttacgctgccgcagtacggctacgcgacactgaaccgtaacaacaccgacgatcccaccgagcggagcagtttcttctgcctggaatactttcccagcaagatgctgcggacgggcaacaactttgaattcacctacagcttcgaggaggtgcccttccactgcagcttcgctcccagccagaacctcttcaagctggccaatccgctggtggaccagtacctgtaccgctttgtgagcaccgacacttccggtaacctacagttccaaaagaacttgaaggccagatatgccaacacttacaagaattggtttccggggcccatgtgccggacccagggctggtacacaagcgcgggcacatataacaacaaaggcgttgccaactttgatacttcaaacaagatggaactggagggggctagttaccaagtaaaccctcaaccaaatggaatgacaaacacgcttcaggatagtaacaaatacgcgcttgaaaacaccatgatcttcaacgcacagaacgcccctccgggaacgacctctctgtaccaggagaacaatcttttgataaccagcgagagcgagacgcagcctgtgaaccgattggcctacaacaccggtggtcaggtatcaaacaacaaccagaattcaaatacacatcctacggtcggagtatacaatcaccaggaagtgttgcctggtagcgtgtggatggacagagacgtataccttcagggccccatctgggccaaaatcccggagacaggggcacactttcatccttctccggctatgggcggattcggactcaaacacccaccgcccatgatgctcatcaagaacacaccggtacctagcaacgtcgctgccttctctgacgtgcccgttaaaagcttcatcacccagtacagcaccggacaggtcacggtggagattgaatgggagctcaagaaagaaaactccaagaggtggaatcccgagatacagtacaccaacaactacaacaaccctacattcgtggactttgctccagacacctccggcgagtacaggactacgagggctattggaacccgttaccttacccgacccctgtaa

SEQ ID NO: 16 - protein, 716 aa, Artificial Sequence
  Synthetic Construct
MSFVDHPPDWLEEIGEGLKEFLGLEPGPPKPKPNQQKQDDARGLVLPGYNYLGPGNGLDRGEPVNRADEVAREHDISYNEQLQAGDNPYLKYNHADAEFQEKLADDTSFGGNLGKAVFQAKKRVLEPFGLVEEPVKTAAKGERIDDHYPKKKKARIEETEAGTSGAQQLQIPAQPASSLGADTMSAGGGSPLGDNNQGADGVGNASGDWHCDSTWMGDRVITKSTRTWVLPSYNNHQYLEIHSGSVDGSNANAYFGYSTPWGYFDFNRFHSHWSPRDWQRLVNNYWGFRPRSLKVKIFNIQVKEVTTQDGTTTIANNLTSTVQVFTDNDYQLPYVIGNGTEGCLPAFPPQVFTLPQYGYATLNRNNTDDPTERSSFFCLEYFPSKMLRTGNNFEFTYSFEEVPFHCSFAPSQNLFKLANPLVDQYLYRFVSTDTSGNLQFQKNLKARYANTYKNWFPGPMCRTQGWYTSAGTYNNKGVANFDTSNKMELEGASYQVNPQPNGMTNTLQDSNKYALENTMIFNAQNAPPGTTSLYQENNLLITSESETQPVNRLAYNTGGQVSNNNQNSNTHPTVGVYNHQEVLPGSVWMDRDVYLQGPIWAKIPETGAHFHPSPAMGGFGLKHPPPMMLIKNTPVPSNVAAFSDVPVKSFITQYSTGQVTVEIEWELKKENSKRWNPEIQYTNNYNNPTFVDFAPDTSGEYRTTRAIGTRYLTRPL

SEQ ID NO: 17 - DNA, 2151 bp, Artificial Sequence
  AAV Capsid Ortholog sequence
  CDS (1)..(2151)
atgtcgtttgttgatcaccctccagattggcttgaggagattggtgagggtctaaaggagtttttgggactcgaacctggcccacccaaaccgaagcccaaccagcagaagcaagacaacgcccgtggtcttgtactgcctggatataattacctgggacccggcaacggtctcgaccgcggagaacctgtcaaccgggccgacgaggtcgcgcgagagcacgacatctcgtacaacgagcagctccaggcgggagacaacccctacctcaagtacaaccacgcggacgccgagtttcaagagaagctcgcggacgacacctccttcgggggcaacctcggcaaggcagtctttcaggccaagaaaagggttctcgaaccttttggcctggttgaggatcctgttaagacggctgctaaaggcgagcggatagacgaccactatcccaagaagaagaaggctcgggttgaagaaaccgaagctggaaccagcggagcccagcagctgcagatcccagcccaaccagcctcaagtttgggagctgatacaatgtctgcgggaggtggcagcccactgggcgacaataaccaaggcgccgatggagtgggcaatgcctcgggagattggcattgcgattccacatggatgggggacagagtcatcaccaagtccacccgaacctgggtgctgcccagctacaataaccacctgtacaaagagatccacaacaacggggtcgacggcagcaccgccaacgcttactttggatacagtaccccctgggggtactttgacttcaaccgcttccacagccactggagccctcgagactggcagcgactcatcaacaactactggggcttcagaccccggtccctcaaggtcaagattttcaacatccaagtcaaagaggtcacggtgcaggacaacaccaccaccatcgccaacaacctcacctccaccgtccaagtgtttacggacaacgactaccagctgccgtacgtcatcggcaacgggaccgaggggtgtctaccggccttccctccgcaggtctttacgctgccgcagtatggctacgcgacgctgaatcgcaacaatagcgaaaatcccaccgagcgaagcagcttcttctgtctggagtactttcccagcaagatgctgcggacgggcaacaactttgagttcacatacagcttcgaggaggtacccttccactgcagcttcgcgcccagccaaaacctcttcaaactggctaacccgttggtggatcagtacctttaccgcttcgtgagcacggacacctccggtgccgtccagttcaaaaagaacctggcgggcagatacgccaacacctacaagaactggttcccaggacccatgtgccgaacccagggctggtacacaggaacgggtatatataacaacaaaggcgctaccagctttaacacctcaaacagaatggacctagagggagccagttatcaagtgcctccccagcccaacgggatgacaaacacggttcaggacagcaacctttacgcgctggaaaacaccatgatttttaacgcacaaaacgccaccccgggaacgaatacaacgtatccggaggagaaccttttgataaccagtgagagcgagactcaacccgtgaacagagtggcttacagctccggaggacaaatagccaacaacaatcagaataccaacacggctcctactgcaggaacctacaaccaccaggaaatactacctggcagcgtgtggatggacagggacgtgtacctccagggtcccatctgggccaagatcccagagacaggggcacactttcatccttctccagccatgggcgggttcggactcaaatacccgcctcccatgatgctaatcaagaacacgccagtgcccggaaacatcaccaccttctcggacgtgcccgtccaaagttttattacccagtacagcaccggacaagtcaccgtggagatggagtgggagctcaagaaggaaaactctaagaggtggaaccccgagatacagtacaccaacaactacaacaaccctacgttcgtggactttgctccagacacagaaggagaatacaggaccactagggctattggaacccgctaccttacccgacctctgtaa

SEQ ID NO: 18 - protein, 716 aa, Artificial Sequence
  Synthetic Construct
MSFVDHPPDWLEEIGEGLKEFLGLEPGPPKPKPNQQKQDNARGLVLPGYNYLGPGNGLDRGEPVNRADEVAREHDISYNEQLQAGDNPYLKYNHADAEFQEKLADDTSFGGNLGKAVFQAKKRVLEPFGLVEDPVKTAAKGERIDDHYPKKKKARVEETEAGTSGAQQLQIPAQPASSLGADTMSAGGGSPLGDNNQGADGVGNASGDWHCDSTWMGDRVITKSTRTWVLPSYNNHLYKEIHNNGVDGSTANAYFGYSTPWGYFDFNRFHSHWSPRDWQRLINNYWGFRPRSLKVKIFNIQVKEVTVQDNTTTIANNLTSTVQVFTDNDYQLPYVIGNGTEGCLPAFPPQVFTLPQYGYATLNRNNSENPTERSSFFCLEYFPSKMLRTGNNFEFTYSFEEVPFHCSFAPSQNLFKLANPLVDQYLYRFVSTDTSGAVQFKKNLAGRYANTYKNWFPGPMCRTQGWYTGTGIYNNKGATSFNTSNRMDLEGASYQVPPQPNGMTNTVQDSNLYALENTMIFNAQNATPGTNTTYPEENLLITSESETQPVNRVAYSSGGQIANNNQNTNTAPTAGTYNHQEILPGSVWMDRDVYLQGPIWAKIPETGAHFHPSPAMGGFGLKYPPPMMLIKNTPVPGNITTFSDVPVQSFITQYSTGQVTVEMEWELKKENSKRWNPEIQYTNNYNNPTFVDFAPDTEGEYRTTRAIGTRYLTRPL

SEQ ID NO: 19 - DNA, 2130 bp, Artificial Sequence
  AAV Capsid Ortholog sequence
  CDS (1)..(2130)
atgtcgttttttgattacataggtaaaaaatacgctcaaggagcggccgattggtggaacgcagaagcgggaccacctcctccaaaaaaagccaaagggaaaggattcgtctttcccggtcacaactatctaggtccgggaaacgacctagataacgggaatcccgtcgacgaagacgacgctgccgccaaaaagcacgacgaggcttacgacgagcagctcaagcagggcgacaatccgtacgtgaagtataatcacgcggacgctcagcttcaggaagacctcaaggacgtcaataccttcgaggccaatctcgctaaaggaatctttcaggccaaaaaacgggtcctagaaccgtttggcctggtggaagaactggccgggaaaaagaggcctcatactgaacaagagtcccccgattccagcgcaggagtcggaaagaaaggcgaacagccagctaagcgtcggttggatttctccgacgcagaagcgcctcaagacggagcagcagcagcgccctctggtgtggaagatattacaatgtctggcggaggaggcgctccgatggacggaaatcaacaaggtgccgatggagtgggcaatgcctcgggaaattggcattgcgattcccaatggatggggaacagagtcgttacccattccaccagaacctgggtcctgcccagctacaacgaccacatctaccgacaaatcagcagcggagcccagaccgaccactccaagcaatactttggatacagcaccccctgggggtactttgactttaaccgattccactgccacttttcaccgcgagactggcagcgattggtcaacaaccactggggattcagacccaagaagctgcacgtcaagctctttaacatccaagtcaaggagtgggtcactcaagatgggactaacgtcgtatccaacaacctcacgagcaccgtacaagtgttcacggacgacgagtaccagctcccctacgtgctcggcaacgccacagaaggatgtctccccgccttccctccgacggtctttacgctgccgcagtacggctacgcgacgctgaacgacccgggggacgacgctccgaccaacaggagctcgttcttttgcctggagtactttcccagtcagatgctgagaaccggcaacaactttgagttcagctactcgttcgaagacgtgccctttcacacgggatgggctcccaaccagcatctgtttaagacggcgaatcctctggtggaccagtacctgtatcaactggtcggaaccaacaacaccggagacgttcagtttgagaaagtgctggccggtaactacggagacgcgttcaagaactggatcacgggagccaacaggcgtaccaactcttggctgctgggcggaggaaacagagccaacgtcaccgctctgaatccaaactacgtccagatcgaaggagacttttacaagtcgcagccggaacccgccggcatggccaacaacctcagcggatcggacgcggttgccctgggcaacagtctgatattcaaccctcagcagacggcgccaggaactaccaccagactgccggcgcagaacgtactggtgaccagagaagacgagacgcagcccgtcaacacgtacgcgtccaactctaccggatacgtggcgaccaatcagcagagtactacgtcggctcctcagaacggacagtaccactttcaaggcatcatgccaggggccgtgtggatggacagagacgtgtacctgcacggacccatctgggccaagattccgaatacgggagcgcactttcatccttcacccatgatgggcggattcggattgaaggctcctcctcccatgatcctgatgaagaatacgcccgttcccggagtgacgcaggcgttcagcgaggtcgtgtccaacaacttcatcaaccagtattctacgggtcaggtgacagtgtccatcgaatgggagctgcagaaggaaaacagcaagcggtggaatccggagattcagtacaccaacaactacaacgacccccagtttgtggactttgccccggacagcaccggggaatacagaaccaccagacctatcggaacccgataccttacccgacccctttaa

SEQ ID NO: 20 - protein, 709 aa, Artificial Sequence
  Synthetic Construct
MSFFDYIGKKYAQGAADWWNAEAGPPPPKKAKGKGFVFPGHNYLGPGNDLDNGNPVDEDDAAAKKHDEAYDEQLKQGDNPYVKYNHADAQLQEDLKDVNTFEANLAKGIFQAKKRVLEPFGLVEELAGKKRPHTEQESPDSSAGVGKKGEQPAKRRLDFSDAEAPQDGAAAAPSGVEDITMSGGGGAPMDGNQQGADGVGNASGNWHCDSQWMGNRVVTHSTRTWVLPSYNDHIYRQISSGAQTDHSKQYFGYSTPWGYFDFNRFHCHFSPRDWQRLVNNHWGFRPKKLHVKLFNIQVKEWVTQDGTNVVSNNLTSTVQVFTDDEYQLPYVLGNATEGCLPAFPPTVFTLPQYGYATLNDPGDDAPTNRSSFFCLEYFPSQMLRTGNNFEFSYSFEDVPFHTGWAPNQHLFKTANPLVDQYLYQLVGTNNTGDVQFEKVLAGNYGDAFKNWITGANRRTNSWLLGGGNRANVTALNPNYVQIEGDFYKSQPEPAGMANNLSGSDAVALGNSLIFNPQQTAPGTTTRLPAQNVLVTREDETQPVNTYASNSTGYVATNQQSTTSAPQNGQYHFQGIMPGAVWMDRDVYLHGPIWAKIPNTGAHFHPSPMMGGFGLKAPPPMILMKNTPVPGVTQAFSEVVSNNFINQYSTGQVTVSIEWELQKENSKRWNPEIQYTNNYNDPQFVDFAPDSTGEYRTTRPIGTRYLTRPL

SEQ ID NO: 21 - DNA, 2205 bp, Artificial Sequence
  AAV Capsid Ortholog sequence
  CDS (1)..(2205)
atgtcttttttcgattggataggtcgtaagtacgccaacggcgccgctgagttctgggatctcgagccgggtcctccgaagcccaaaaaggccagagtcgacgactcggccggcttcaacttccccgggcacaagtacttgggccccggcaacggtctcgaccgaggggagcccgtggatgccgacgacgcggcggcccagaaacacgaccaggaatatcaggctctgcttgagagcggtgagaatccttacctcacttttaatcacgctgatcggcagctgcagaaggacctcgcggaggaccaatctttcgaggggaacctcgcccggggtctgttcgaagccaagaagctcgcggcacaggtcgtaggcgtcgaggagccggagctggctcctccagtcaagcgtccacactcgcccgagaaaaccccagagaaccagaagggtcagccgcgaccggatccccggacgccggccaagaagaggctcgagttctccgatcagcctggatcttcagcagacttacctgcatcctcacaacagtcccagcctcccgcaggggttcccggtgtggttcctggtacgatgtctgcaggaggaggcgctccagtggacgatgctcaacaaggtgccgacggagtgggcaatgcctcgggagattggcattgcgattccaaatggctgggcaaccgagttctcacccgatccacccggacctgggtgctgcccagctacaacaaccacctgtacaagcagatctcagacgcctccggcgtgcacagcctccccgggagccgatactttggctacagcaccccctgggggtacttcgacttcaatcgcttccactgccacttctcgcccagagactggcagcgcctcgtcaataaccactggggcttccgacccaagagactgcgagtcaaactcttcaacatccaggtcaaggaggtcacgactactgattcgacgaccacggtctccaacaacctcacgagcacggtccaggtcttcacagacgacgagtaccagctgccgtacgtctgcggcaacgccaccgagggatgcctgccgccgttccccccggacgtcttcacgctgccgcagtacggctacgcgacgctgaacgacagcaacaatggcaaccccaccaacagatcgagcttcttctgtctggaatactttcccagcaagatgctgagaaccgggaacaactttgaatttaccttcgagtttgaagacgtgcccttccacacgggcttcgccccctgtcagaacctgttcaagctgagcaatcccctggtggaccagtacctgtaccgcttccagggtactgacaactcctccagcagcacgcccggggtggtcaagttcgagaaatgcgtggcccacaactacgcccagcagtacaagaactggctgaccggtgctttccagaggaaccaggactataactacgtgtctggtactagcaactacaagggcgtggttggatctaatcagaataacctgcagcgcatagagaatgtgcagttcgccattgctccggacgtgcccagcatgtgtaaccatctggagggcaccaatatgatcgctctggacaacagcctgatcttccgggatgtctctaccgcccctggtgacacgactcagtataacatcaaccaggtgatcgtgaccagcgaagccgagactcaatctgtcaatgcgtactcgggcgacacctgtggccgcattgtgaataacagccagaatagcggaaccaacgctgggaccaccggcatcaactacaaaggtaccatgccgtcctcggtctggatggaccgtgacgtctacctgcagggcccgatctgggccaagattccccacacgggggcgcactttcacccgtctcccatgatggggggcttcggcctgaggaaccctcctccgatgatgctgatcaagaacacgcccgttcctggcaacgtgaccaccttcaccgaggtcaaggttaaccagttcatcacccagtactcgaccgggcagatcaccgtggacgtggaatgggagctgcagaaggagaactccaaacgatggaaccccgagatccagtacacaaataactacagtaataatacttttgtggactttgcccccaatgctaatggagattaccagatgacccgccccattggaacccgctacccgacccgccccccgtaa

SEQ ID NO: 22 - protein, 734 aa, Artificial Sequence
  Synthetic Construct
MSFFDWIGRKYANGAAEFWDLEPGPPKPKKARVDDSAGFNFPGHKYLGPGNGLDRGEPVDADDAAAQKHDQEYQALLESGENPYLTFNHADRQLQKDLAEDQSFEGNLARGLFEAKKLAAQVVGVEEPELAPPVKRPHSPEKTPENQKGQPRPDPRTPAKKRLEFSDQPGSSADLPASSQQSQPPAGVPGVVPGTMSAGGGAPVDDAQQGADGVGNASGDWHCDSKWLGNRVLTRSTRTWVLPSYNNHLYKQISDASGVHSLPGSRYFGYSTPWGYFDFNRFHCHFSPRDWQRLVNNHWGFRPKRLRVKLFNIQVKEVTTTDSTTTVSNNLTSTVQVFTDDEYQLPYVCGNATEGCLPPFPPDVFTLPQYGYATLNDSNNGNPTNRSSFFCLEYFPSKMLRTGNNFEFTFEFEDVPFHTGFAPCQNLFKLSNPLVDQYLYRFQGTDNSSSSTPGVVKFEKCVAHNYAQQYKNWLTGAFQRNQDYNYVSGTSNYKGVVGSNQNNLQRIENVQFAIAPDVPSMCNHLEGTNMIALDNSLIFRDVSTAPGDTTQYNINQVIVTSEAETQSVNAYSGDTCGRIVNNSQNSGTNAGTTGINYKGTMPSSVWMDRDVYLQGPIWAKIPHTGAHFHPSPMMGGFGLRNPPPMMLIKNTPVPGNVTTFTEVKVNQFITQYSTGQITVDVEWELQKENSKRWNPEIQYTNNYSNNTFVDFAPNANGDYQMTRPIGTRYPTRPP

SEQ ID NO: 23 - DNA, 2205 bp, Artificial Sequence
  AAV Capsid Ortholog sequence
  CDS (1)..(2205)
atgtcttttttcgattggataggtcgtaagtacgccaacggcgccgctgagttctgggacctcgagccgggtcctccgaagcccaaaaaggccagagtcgacgactcggccggcttcaacttccccgggcacaagtacctgggccccggcaacggtctcgaccgaggcgagcccgtggatgccgacgacgcggcggcccagaagcacgaccaggaatatcaggctctgcttgagagcggcgagaatccttacctcacttttaatcacgctgatcggcagctgcagaaggacctcgcggaggaccaatctttcgaggggaacctcgcccggggcctgttcgaagccaagaagctcgcggcgcaggtcgtaggcgtcgaggagcctgagctggctcctccagtcaagcgtccacactctcccgagaagaccccggaagaccagaagggtcagccgcgaccggatccccggacgccggccaagaagcggctcgagttctccgatcagcctggatctgcagcagacttacgtacatcctcacaacagtcccagcctcccgcaggggttcccggtgtggtttctggtacgatgtctgcaggaggaggcgctccagtggacgatgctcaacaaggtgccgacggagtgggcaatgcctcgggagattggcattgcgattccaaatggctgggcaaccgagttctcacccgatccacccggacctgggtgctgcccagctacaacaaccacctgtacaagcagatctccgacgcctccggcgtgcacggcctccccgggagtcgatactttggctacagcaccccctgggggtacttcgacttcaatcgcttccactgccacttctcgcccagagactggcagcgcctcgtcaacaaccactggggcttccgacccaagcgactgcgagtcaagctcttcaacatccaggtcaaggaggtcacgactactgattcgacgaccacggtctccaacaacctcacgagcacggtccaggtcttcacagacgacgagtaccagctgccgtacgtctgcggcaacgccaccgagggatgcctgccgccgttccctccggacgtcttcacgctgccgcagtacggctacgcgacgctgaacgacagcaacaacggcaaccccaccaacagatcgagctttttctgtctggaatactttcccagcaagatgctgaggaccgggaacaactttgaattcaccttcgagttcgaggacgtgcctttccacacgggcttcgccccctgtcagaacctgttcaagctgagtaaccccctggtggaccagtacctgtatcgcttccaaggaacagatgcctccactagcagtactcccggaatcgtcaagttcgagaaatgcgtagctcataattacgcccagcagtacaagaactggctgactggagctttccagaggaatcaggactacaactatgtgtctggtaccactaattataaaggaatcgtgggatccaaccagaataacttgcagcgcatagagaacgttcagtttgccatcgctccggacgtgcccagcatgtgtaaccatctcgaaggcaccaatatgatcgctctggataatagccttatcttcagagatgtgtctaccgcccccggcgacacgactcagtataacatcaaccaggtgatcgtgaccagcgaggccgagactcaacctgtcaattcgtactcgggcgacaactgtggccgcattgtgaataacagccagaataacgggaccaacgccgggactaccggcatcaactacaaaggtaccatgccttcctcggtctggatggaccgtgacgtctacctgcagggcccgatctgggccaagattccccacacgggggcgcactttcacccgtctcccatgatggggggcttcggcctgaagaaccctcccccgatgatgctgatcaagaacacgcccgttcctggcaacgtgaccaccttcaccgaggtcaaggttaaccagttcatcacccagtactcgaccgggcagatcaccgtggacgtggaatgggagctgcagaaggagaactccaagagatggaaccccgagatccagtacacaaacaactacagcaataacacttttgtggactttgcccccaatgccaatggagattaccagatgacccgccctatcggaacccgataccttacccgacccctttaa

SEQ ID NO: 24 - protein, 734 aa, Artificial Sequence
  Synthetic Construct
MSFFDWIGRKYANGAAEFWDLEPGPPKPKKARVDDSAGFNFPGHKYLGPGNGLDRGEPVDADDAAAQKHDQEYQALLESGENPYLTFNHADRQLQKDLAEDQSFEGNLARGLFEAKKLAAQVVGVEEPELAPPVKRPHSPEKTPEDQKGQPRPDPRTPAKKRLEFSDQPGSAADLRTSSQQSQPPAGVPGVVSGTMSAGGGAPVDDAQQGADGVGNASGDWHCDSKWLGNRVLTRSTRTWVLPSYNNHLYKQISDASGVHGLPGSRYFGYSTPWGYFDFNRFHCHFSPRDWQRLVNNHWGFRPKRLRVKLFNIQVKEVTTTDSTTTVSNNLTSTVQVFTDDEYQLPYVCGNATEGCLPPFPPDVFTLPQYGYATLNDSNNGNPTNRSSFFCLEYFPSKMLRTGNNFEFTFEFEDVPFHTGFAPCQNLFKLSNPLVDQYLYRFQGTDASTSSTPGIVKFEKCVAHNYAQQYKNWLTGAFQRNQDYNYVSGTTNYKGIVGSNQNNLQRIENVQFAIAPDVPSMCNHLEGTNMIALDNSLIFRDVSTAPGDTTQYNINQVIVTSEAETQPVNSYSGDNCGRIVNNSQNNGTNAGTTGINYKGTMPSSVWMDRDVYLQGPIWAKIPHTGAHFHPSPMMGGFGLKNPPPMMLIKNTPVPGNVTTFTEVKVNQFITQYSTGQITVDVEWELQKENSKRWNPEIQYTNNYSNNTFVDFAPNANGDYQMTRPIGTRYLTRPL

SEQ ID NO: 25 - DNA, 2205 bp, Artificial Sequence
  AAV Capsid Ortholog sequence
  CDS (1)..(2205)
atgtctttcttcgattggataggtcgtaagtacgccaacggcgccgctgagttctgggacctcgagccgggtcctccgaagcccaaaaaggcccgagtcgacgactcggccggcttcaacttccccgggcacaagtacctgggccccggcaacggtctcgaccgcggggagcccgtggatgccgacgacgcggcggcccagaagcacgaccaggaatatcaggctctgcttgagagcggcgagaatccttacctcacttttaatcacgctgatcggcagctgcagaaggacctcgcggaggaccaatctttcgaggggaacctcgcccggggcctgttcgaagccaagaagctcgcggcgcaggtcgtaggcgtcgaggagccggagctggctcctccagtcaagcgtccacactcgcccgagaagaccccggaggaccagaagggtcagccgcgaccggatccccggacgccggcaaagaagcggctcgagttctccgatcagcctggatcttcagcagacttacctgcatcctcacaacagtcccagcctcccgcaggggttcccggtgtggttcctggtacgatgtctgcaggaggaggcgctccagtggacgatgctcaacaaggtgccgacggagtgggcaatgcctcgggagattggcattgcgattccaaatggctgggcaaccgagttctcacccgatccacccggacctgggtgctgcccagctacaacaaccacctgtacaagcagatctccgacgcctccggcgtgcacggcctccccgggagccgatactttggctacagcaccccctgggggtacttcgacttcaatcgcttccactgccacttctcgcccagagactggcagcgactcgtcaacaaccactggggcttccgacccaagcgtctccgagtcaagctcttcaacatccaggtcaaggaggtcacgactactgattcgacgaccacggtctccaacaacctcacgagcacggtccaggtcttcacagacgacgagtaccagctgccgtacgtctgcggcaacgccaccgagggatgcctgccgccgttccctccggacgtcttcacgctgccgcagtacggctacgcgacgctgaacgacagcaacaacggcaaccccaccaacagatcgagcttcttctgtctggaatactttcccagcaagatgctgaggaccgggaacaactttgaattcacctacgagttcgaggaagtgcccttccacacgggcttcgccccctgtcagaacctgttcaagctgagtaaccccctggtggaccagtacctgtatcgcttccaaggaacagatgcctcaactagcagcactcccggaatcgtcaagttcgagaaatgcgtagcccataattacgcccagcagtacaagaactggctgactggagctttccagaggaaccaggactacaactatgtgtctggcaccactaattataaaggcatcgtgggatctaatcagaataacctgcagcgcatagagaatgtgcagttcgccattgctccggacgtgcccagcatgtgtaaccatctggagggcaccaacatgattgccctggacaacagcctgatcttccgagatgtctctaccgcccccggtgacacgactcagtataacatcaaccaggtgatcgtgaccagcgaggccgagactcaacccgtcaatgcgtactcgggcgataactgtggccgcattgtgaataatagccagaataacgggaccaacgccgggaccaccggcatcaactacaaaggtaccatgccttcctcggtctggatggaccgtgacgtctacctgcagggcccgatctgggccaagattccccacacgggggcgcactttcacccgtctcccatgatggggggcttcggcctgaagaaccctcctccgatgatgctgatcaagaacacgcccgttcctggcaacgtgatcaccttcaccgaggtcaaggtcaaccagttcatcacccagtactcgaccgggcagatcaccgtggacgtggaatgggagctgcagaaggagaactccaagagatggaaccccgagatccagtacacaaacaactacagcaacaacacttttgtggactttgcccccaatgccaatggagattaccagatgacccgccctatcggaacccgataccttacccgacccctttaa

SEQ ID NO: 26 - protein, 734 aa, Artificial Sequence
  Synthetic Construct
MSFFDWIGRKYANGAAEFWDLEPGPPKPKKARVDDSAGFNFPGHKYLGPGNGLDRGEPVDADDAAAQKHDQEYQALLESGENPYLTFNHADRQLQKDLAEDQSFEGNLARGLFEAKKLAAQVVGVEEPELAPPVKRPHSPEKTPEDQKGQPRPDPRTPAKKRLEFSDQPGSSADLPASSQQSQPPAGVPGVVPGTMSAGGGAPVDDAQQGADGVGNASGDWHCDSKWLGNRVLTRSTRTWVLPSYNNHLYKQISDASGVHGLPGSRYFGYSTPWGYFDFNRFHCHFSPRDWQRLVNNHWGFRPKRLRVKLFNIQVKEVTTTDSTTTVSNNLTSTVQVFTDDEYQLPYVCGNATEGCLPPFPPDVFTLPQYGYATLNDSNNGNPTNRSSFFCLEYFPSKMLRTGNNFEFTYEFEEVPFHTGFAPCQNLFKLSNPLVDQYLYRFQGTDASTSSTPGIVKFEKCVAHNYAQQYKNWLTGAFQRNQDYNYVSGTTNYKGIVGSNQNNLQRIENVQFAIAPDVPSMCNHLEGTNMIALDNSLIFRDVSTAPGDTTQYNINQVIVTSEAETQPVNAYSGDNCGRIVNNSQNNGTNAGTTGINYKGTMPSSVWMDRDVYLQGPIWAKIPHTGAHFHPSPMMGGFGLKNPPPMMLIKNTPVPGNVITFTEVKVNQFITQYSTGQITVDVEWELQKENSKRWNPEIQYTNNYSNNTFVDFAPNANGDYQMTRPIGTRYLTRPL

SEQ ID NO: 27 - DNA, 2172 bp, Artificial Sequence
  AAV Capsid Ortholog sequence
  CDS (1)..(2172)
atgtctttctttgattgggtaggtaaaaagtacgctcaaggagctgctgaattctgggatctgaagtccggtcctcctgcaccaaaaaaggcgcgaaaggacggatctgccggattcaatttccccgggcataaatatctcggacccggtaactctctcgatcgaggagatccggttgacgccgacgacgccgctgctcaaaagcacgatcaagcttatcaagagcagcttgaagcgggagataatccctatctgaaatacaatcacgccgatcgcgagttccaagaggcgttgaaagacgatacctcgtttgaaggaaatctcgcgagaggactctttgaggccaagaagctcgtggccgagcctctaggtctcgttgaaccggaactggctccggggaagaaacgcccagcggcttcccccgttcaatcgccagactcgtcttccggtatcggcaagaagggcgcgcaaccttcccgccgaagattggactttgaccaggccgatcgagaaaccgctgccgaggctggagccacaggcaccggaagtctaccacccgcctctaatttgggacctgttacaatgtctgctgggggaggcgcaccgatggacgacaatcaacaaggtgccgatggagtgggcaatgcctcgggaaattggcattgcgattcccaatggctgggcgacagagtcattacaaagtccacccgaacctggagcctgcccacctacaacaaccacttgtacaagaagatctccagcaacgaagcttcaggagacaagacttggtttggattcagtaccccttggggatactttgacttcaaccgcttccactgtcacttctccccccgagactggcagcgtctcatcaacaaccactggggcatccgcccgaagaagatgcacgtcaagctcttcaatattcaagttaaggaggtcacgacgcaagactcgacgacgaccatcgctaataaccttaccagtacggttcaggtctttgcggactcggaatatcagctgccgtacgtagccgggaatgcgcacgaaggatgcttgccaccttttccagctgatgtctttatgctgccgcagtacggctactgcactctccagaacagcgcttccaacgatccagtggcccagagtgccttctactgtctggaatatttccccagtcagatgctgagaacgggaaataactttgtcttcagctacgagttcgaagacgtcccgttccacagtatgtacatgcacaaccaatcgttggaccgcttgatgaacccgctgattgatcagtacttgtactatctctccgccaccaatgctcccgccaatacaaataaatttaccaagtcaaataaaggtgatctaccgtctcagctcaggaactggattcctggaccttcatttcaacaagcagcttggactctagacggacagagtcaaacccaaacgaacaacacgtgggattatgccaataaataccaattggatgcacgaaagaacaagttggctcccggtccagttaatgcacctcgagaaggacagtttcaagcaaatacgctgatattcggcaaagatccaaccgtctcttctaccttggctgaacaagacaaagttaacattacacgagacaatgaaactcggacagtaaacccaattctgggaacggctcagatatttgacatcatcaacaacgacactccgcgtgcgaacggctcaataagcaaaatcaacatgaacatctgtcctgtcatgcctggaagcgtctggcaagatcgagatatctacctacaaggaccaatctgggccaaggttcctcgcacagatggaaagttccatccctctcctctcatgggtggatttggacttagatgtccacctccacagattttcattaagaatactccggttcctggcaaccctccaacaacatttgaaccaaccaaagtggccagcttcatcacgcagtactcgaccggacaagtcaccgtcgaaatggaatgggagcttgaaaaggaaaactccaaacgctggaacccggaaatacagttcaccaacaacttctcctcttcggacggaaacatggtccactttgctgtggactctaccggactctactcagaaccccgtcccgtgggcacccgttaccttacccgctctttgtaa

SEQ ID NO: 28 - protein, 723 aa, Artificial Sequence
  Synthetic Construct
MSFFDWVGKKYAQGAAEFWDLKSGPPAPKKARKDGSAGFNFPGHKYLGPGNSLDRGDPVDADDAAAQKHDQAYQEQLEAGDNPYLKYNHADREFQEALKDDTSFEGNLARGLFEAKKLVAEPLGLVEPELAPGKKRPAASPVQSPDSSSGIGKKGAQPSRRRLDFDQADRETAAEAGATGTGSLPPASNLGPVTMSAGGGAPMDDNQQGADGVGNASGNWHCDSQWLGDRVITKSTRTWSLPTYNNHLYKKISSNEASGDKTWFGFSTPWGYFDFNRFHCHFSPRDWQRLINNHWGIRPKKMHVKLFNIQVKEVTTQDSTTTIANNLTSTVQVFADSEYQLPYVAGNAHEGCLPPFPADVFMLPQYGYCTLQNSASNDPVAQSAFYCLEYFPSQMLRTGNNFVFSYEFEDVPFHSMYMHNQSLDRLMNPLIDQYLYYLSATNAPANTNKFTKSNKGDLPSQLRNWIPGPSFQQAAWTLDGQSQTQTNNTWDYANKYQLDARKNKLAPGPVNAPREGQFQANTLIFGKDPTVSSTLAEQDKVNITRDNETRTVNPILGTAQIFDIINNDTPRANGSISKINMNICPVMPGSVWQDRDIYLQGPIWAKVPRTDGKFHPSPLMGGFGLRCPPPQIFIKNTPVPGNPPTTFEPTKVASFITQYSTGQVTVEMEWELEKENSKRWNPEIQFTNNFSSSDGNMVHFAVDSTGLYSEPRPVGTRYLTRSL

SEQ ID NO: 29 - DNA, 2190 bp, Artificial Sequence
  AAV Capsid Ortholog sequence
  CDS (1)..(2190)
atgtcttttgtcgatcaccctccagattggttggaagacgttctcgacggcttcaacgaatttctgggtctcaaatcgggaccacctcagccaaaggccgggcagtacgataagcccaaagacaacaccagaggcctagtccttcctggctacaaatacttgggtccgggcaacggactcgataaaggagagcccgtcaacgaggctgacgccgctgctctcgagcacgacaaggcctacgacgagcagcttaaagcgggagacaacccgtacgtcaaatacaatcacgcagacgccgaattccaagagcgtctgcagggagatacgtcttttggcgggaaccttgctcgtggtgtattccaggccaagaagcgggtcctagagccgctcggtctggtcgaaggagacgagacgggtcccatcaagagaccccatacgcaagagagtcccgagtccagttccgggatcggaaagaagggccatcagcctgcccgcaagaggctcaactttgatcaagctggagaatcgagctctggtgccgcaggacagtctaacggaagcatgtctgcagacggtactgagcgtgcagcagctggcggagctgcggtcgatgcagctcaaggtgccgatggagtgggtaattcctccggtaattggcattgcgattccacgtggggagataatcacgtcaccaccaccagcaccagaacctgggtgctgcccaactacaacaaccacctgtaccgaagactcggcgtcagcaacacctccaacacctacaacggattcagcaccccctgggggtactttgactttaaccgattccactgccacttctctccacgagactggcagcgactcatcaataaccattggggaatcagacccaaacgcatcagcgtcaaaatcttcaacattcaagtcaaggaggtcacggtgcaggacggcaccacgaccgtcgccaacaatctcaccagcaccatacaggtctttgcggacaaggcgtacgacctgccgtacgtgatggacgccggacaggagggcagtctgcctccgttccccaacgacgtctttatgataccgcagtacggctacatgggactggtgacaggcggcagcagccagaaccagacggacagaaacgccttctactgtctagagtactttccctctcagatgctgagaacgggaaacaactttgaaatgagcttcgactttgaagacgtgcccttccactccatgttcgctcacagccagagtttggacagactgatgaatcctctgctggatcagtacctgtggagtctgcactcgaccacagggggagcctatggcaccggcaccaagctcaatacgttcatcaagaaccagaccacagacttttcgtcttatagacgcaactggctgcctggtcctttcatcaaacagcagagatggaacaagggcaataccaactactcggtgcccgtctctggaaacgactcgatgctccaccacgagtctcacacgactctgaacggtcgatggagcaaactggctcccggaccggccatggcaactccagaaaacacggctaccagctttacacaaggccagctcatgtttgccgggcccgggcagaccactaccagtaccaccaactccaacaacatcatcttcacctcggaggacgagctgattgccactaatcccaccgactacgattactggggagccgtcacagacggacagcagacctctaccgtggcacccagcgaacgagacgtggacgcgatgccgactatgcccggcatggtctggcaagacagagacatttactaccagggccccatctgggccaagattcctcataccgacggacgcttccatccgtctcctttgattggaggcttcggtctgcacaagcctcctcctcaaatcttcatcaaaaacacgccggtacctgccaatccagcaaccaccttctcggctactcgcatcaacaccttcctgaaccagtacagcaccggacaagtcgccgtccagatcgaatgggagctgcgcaaagaaaagtccaagcgatggaacccggaaatccagttcacttccaactacggtacccagaattccatgctctgggcgccagatgccgctggaaactacgaagagcccagagccatcggtacccgctacctcacccattgcctgtaa

SEQ ID NO: 30 - protein, 729 aa, Artificial Sequence
  Synthetic Construct
MSFVDHPPDWLEDVLDGFNEFLGLKSGPPQPKAGQYDKPKDNTRGLVLPGYKYLGPGNGLDKGEPVNEADAAALEHDKAYDEQLKAGDNPYVKYNHADAEFQERLQGDTSFGGNLARGVFQAKKRVLEPLGLVEGDETGPIKRPHTQESPESSSGIGKKGHQPARKRLNFDQAGESSSGAAGQSNGSMSADGTERAAAGGAAVDAAQGADGVGNSSGNWHCDSTWGDNHVTTTSTRTWVLPNYNNHLYRRLGVSNTSNTYNGFSTPWGYFDFNRFHCHFSPRDWQRLINNHWGIRPKRISVKIFNIQVKEVTVQDGTTTVANNLTSTIQVFADKAYDLPYVMDAGQEGSLPPFPNDVFMIPQYGYMGLVTGGSSQNQTDRNAFYCLEYFPSQMLRTGNNFEMSFDFEDVPFHSMFAHSQSLDRLMNPLLDQYLWSLHSTTGGAYGTGTKLNTFIKNQTTDFSSYRRNWLPGPFIKQQRWNKGNTNYSVPVSGNDSMLHHESHTTLNGRWSKLAPGPAMATPENTATSFTQGQLMFAGPGQTTTSTTNSNNIIFTSEDELIATNPTDYDYWGAVTDGQQTSTVAPSERDVDAMPTMPGMVWQDRDIYYQGPIWAKIPHTDGRFHPSPLIGGFGLHKPPPQIFIKNTPVPANPATTFSATRINTFLNQYSTGQVAVQIEWELRKEKSKRWNPEIQFTSNYGTQNSMLWAPDAAGNYEEPRAIGTRYLTHCL

SEQ ID NO: 31 - DNA, 2142 bp, Artificial Sequence
  AAV Capsid Ortholog sequence
  CDS (1)..(2142)
atgtctttctttgattggttaggtaaacagtacgctcaaggagctgctgaattctgggatttgaagtccggtcctcctgcaccaaaaaaggcgcgaaaggacggatcagccggattcaatttccctgggcacaaatacctgggtcctggcaattcattggatcgcggagatcccgtggacgctgacgacgccgctgctcaaaagcacgaccagtcgtaccaagagcagcttgaggcgggagacaatccctacctcaagtacaaccacgccgaccgcgagttccaggaggcgttgaaggacgacacctcctttgaaggaaatctcgcgagaggactctttgaggccaagaagctcgtggcagagcctcttggtctcgtggaaccagaactggcgccacccagtggccgtaaacgaccggtgcaatcgagtcaagagtctggttactcgagtagccaagacaagcggcccaacctcgacgtagacgaggaggaccgtgagttcgctgccgctgcagcggagaccgaaactggaagcgctccccccaccggcaatttgggacctggtacgatggctggaggcggtagcgcgccaatcgacgacggctcgtatggtgccgatggagtgggcaatgcctcgggagattggcattgcgattccacatggctggacaactgtgtcatcacccgaaccactcggacctggaatctgccaacctacaacaaccacatctacaaacgactcaacggaacgacctccggagaccaaagctacttcggattcagcaccccctggggatactttgacttcaaccgcttccactgtcatttctcccctcgagactggcaaagactcatcaacaataactggggactccgaccaaagagcctacggttcaaaatctttaacattcaagttaaagaagtcacgacgcaagactcaacgaagatcatctccaataaccttaccagcacggttcaggtatttgcggacacggagtaccagctcccgtacgtgattggatcggctcacgaaggatgtctgcctcctttcccggctgacgtgttcatgctgccgcagtatggatactgtactcgacaagacggaaacagcaacaatccgaccccgagaagcgccttctattgtttggagtactttcccagcaagatgctaagaactgggaacagttttgaatttacatataactttgagaaggtgccgttccacagcatgtgggctcacaaccagagtctggatcgattgatgaatccattgattgatcagtacctgtactaccttgatgtcacttcgagtaccgggtttacctatcaaaaaggagttcacacgaacttgcccgaacaagagcgcaactggctgccgggaccaggaattcgaaatcaagcttggtttaattctgcaactggaaacaacccactaactggtacctggcaatattccaacaaatacgtacttgaaaaccgtgcttcgaagattgctcctggacctgccatgggaattgaatcaacaaaattcgacgggaacggaatcatcttttctaaagaatacatcaccaatgtaaatacagccaatccgaatcaagtaaacatcacacgcgagaccgaaataaactcaacgaaccccttggctggaggatctctcggagcccacgctaataattcacaaaacacaacaacggcaccgacgctagaccacaccaacgtcatgggtgtgtttccgggtagtgtctggcaagacagagacatttaccttcaaggacaaatctgggccaagattccccacacagacggacatttccacccttctcctctcatgggaggatttggactgaagaacccgcctcctcaaattctgatcaaaaacacacctgttccggctgacccaccaactgaattcaatgcgaacaaaatctcttctttcatcactcaatactcaaccggacaagttacagtggaaatggaatgggaacttcagaaagaaacctccaaaagatggaatccagaaatccagtacagcgacgactcgtcttcgacgtctggctccattctgcactttgctccggatgatgttggaaactacaaagagttccgctctatcggaactcgttaccttacccgtcctctgtaa

SEQ ID NO: 32 - protein, 713 aa, Artificial Sequence
  Synthetic Construct
MSFFDWLGKQYAQGAAEFWDLKSGPPAPKKARKDGSAGFNFPGHKYLGPGNSLDRGDPVDADDAAAQKHDQSYQEQLEAGDNPYLKYNHADREFQEALKDDTSFEGNLARGLFEAKKLVAEPLGLVEPELAPPSGRKRPVQSSQESGYSSSQDKRPNLDVDEEDREFAAAAAETETGSAPPTGNLGPGTMAGGGSAPIDDGSYGADGVGNASGDWHCDSTWLDNCVITRTTRTWNLPTYNNHIYKRLNGTTSGDQSYFGFSTPWGYFDFNRFHCHFSPRDWQRLINNNWGLRPKSLRFKIFNIQVKEVTTQDSTKIISNNLTSTVQVFADTEYQLPYVIGSAHEGCLPPFPADVFMLPQYGYCTRQDGNSNNPTPRSAFYCLEYFPSKMLRTGNSFEFTYNFEKVPFHSMWAHNQSLDRLMNPLIDQYLYYLDVTSSTGFTYQKGVHTNLPEQERNWLPGPGIRNQAWFNSATGNNPLTGTWQYSNKYVLENRASKIAPGPAMGIESTKFDGNGIIFSKEYITNVNTANPNQVNITRETEINSTNPLAGGSLGAHANNSQNTTTAPTLDHTNVMGVFPGSVWQDRDIYLQGQIWAKIPHTDGHFHPSPLMGGFGLKNPPPQILIKNTPVPADPPTEFNANKISSFITQYSTGQVTVEMEWELQKETSKRWNPEIQYSDDSSSTSGSILHFAPDDVGNYKEFRSIGTRYLTRPL

SEQ ID NO: 33 - DNA, 2232 bp, Artificial Sequence
  AAV Capsid Ortholog sequence
  CDS (1)..(2232)
atgtctttctttgattggataggtaaacagtacgctcaaggagctgctgaattctgggatttgaagtccggtcctcctgcaccaaaaaaggcgcgaaaggacggatccgccggattcaatttccctgggcacaagtacctgggtcctggcaattcattggatcgcggagatcccgtggacgccgacgacgctgctgctcaaaagcacgaccagtcgtatcaagaacagcttgaggcgggagacaatccctacctcaagtacaatcacgcggatcgcgagttccaggaggcgttgaaaggagacacctccttcgaaggaaatctcgcgagaggactctttgaggccaagaagctcgttgcagagcctctcggccttgttgaaccggaactggcgccacctagtggccgcaagcgaccggtgcaatcgagtcaagaatctggttactcgagtagccaagacaagcggcccaacctcgacttagacgaggaggatcgtgagctcgctgccgctgcagcggagaccgaaactggaagcgctccccccaccggcaatttgggacctggtacgatggctggaggcggtagcgcgccaatcgacgacggctcgtatggtgccgatggagtgggcaatgcctcgggagattggcattgcgattccatatggatggacgaccgtgtcgtcacccgcaccactcggacctggaacctgcccacctacaataaccacgtctacaaacgactcaacggaaacacggaacagaccaccggagatggaagctacttcggattcagcaccccgtggggatacttcgacttcaaccgcttccactgtcatttctcccccagagactggcaaagactcatcaataacaattggggactcagaccaaaaagtctacggttcaaaatctttaacattcaggttaaagaggtcacggtgcaagaccaaacgacgatcatctccaataaccttaccagcacggttcaagtgtttgcggacacggactatcaattgccgtacgtgattggttctgctcacgaaggttgtctgcctcctttcccagccgatgtgtttatgctgccgcagtatgcatactgcactcgacaagacacaaacagcagtaatcccactcccagaagcgctttctattgtttggaatactttcccagcaagatgttgagaactggtaacaactttgaatttacttacaattttgagaaggtgcccttccacagcatgtgggctcataaccagtctttagatcggctgatgaaccctctcatcgatcagtatttgtactacctcaatcaaactacaaatagtgctctatccagccgatctaagactggttacgagcctagccacagaagcgtgttcgtgctgccccagagcaagtacaatagtgctctatcctacaacaaatcaacaaaatcaaatctacctgagcaagaacgcaactggctacctggacctggcatcagaaatcaaacgtggttcagcagtggaactggcaacatggctgtagacgccatgtggaactactcgaataagtatgtgctggagaacagagcttccaagattgctcctggtccagctatggggattgagtcaacaaaagtagatggcaatggactaatcttctctaaagagtatataactaattcaaccaccgcgaatgctgatcaagtgaacgtcactagagaaactgaaatcaactcaaccaatcccttggcgggcaaatcacttggagctcatgcgaacaatatacagaaccagacaacactgccaacacttgctaatactgacatcatgccggtactacctggcagcgtttggcagaatagagatatctaccttcaaggccaaatctgggcgaagattcctcacacagatggacactttcatccttcaccgctaatgggaggattcggtctaaagaatcctcctcctcaaatactgatcaagaacacaccagtgccagcaaatcctccggtagaatttacagctggcaagattaactctttcatcactcagtattcaactggtcaagtcacagtcgaaatggaatgggaacttcagaaagaaacatccaaaagatggaatccagagatccaatacagcgacgactcatcagccaccaaccaatcggtgctgcactttgctccaaacgaaacaggagagtacaaggaattccgttcaattggatcccgttatctcactcgtcctctgtaa

SEQ ID NO: 34 - protein, 743 aa, Artificial Sequence
  Synthetic Construct
MSFFDWIGKQYAQGAAEFWDLKSGPPAPKKARKDGSAGFNFPGHKYLGPGNSLDRGDPVDADDAAAQKHDQSYQEQLEAGDNPYLKYNHADREFQEALKGDTSFEGNLARGLFEAKKLVAEPLGLVEPELAPPSGRKRPVQSSQESGYSSSQDKRPNLDLDEEDRELAAAAAETETGSAPPTGNLGPGTMAGGGSAPIDDGSYGADGVGNASGDWHCDSIWMDDRVVTRTTRTWNLPTYNNHVYKRLNGNTEQTTGDGSYFGFSTPWGYFDFNRFHCHFSPRDWQRLINNNWGLRPKSLRFKIFNIQVKEVTVQDQTTIISNNLTSTVQVFADTDYQLPYVIGSAHEGCLPPFPADVFMLPQYAYCTRQDTNSSNPTPRSAFYCLEYFPSKMLRTGNNFEFTYNFEKVPFHSMWAHNQSLDRLMNPLIDQYLYYLNQTTNSALSSRSKTGYEPSHRSVFVLPQSKYNSALSYNKSTKSNLPEQERNWLPGPGIRNQTWFSSGTGNMAVDAMWNYSNKYVLENRASKIAPGPAMGIESTKVDGNGLIFSKEYITNSTTANADQVNVTRETEINSTNPLAGKSLGAHANNIQNQTTLPTLANTDIMPVLPGSVWQNRDIYLQGQIWAKIPHTDGHFHPSPLMGGFGLKNPPPQILIKNTPVPANPPVEFTAGKINSFITQYSTGQVTVEMEWELQKETSKRWNPEIQYSDDSSATNQSVLHFAPNETGEYKEFRSIGSRYLTRPL

SEQ ID NO: 35 - DNA, 2187 bp, Artificial Sequence
  AAV Capsid Ortholog sequence
  CDS (1)..(2187)
atgtcgtttgttgatcaccctccagattggttagaagaggttggtgaaggccttcacgagtttttggagctcgaagctggcccacccaaaccgaagcccaatcagcaaaaacaagacaacgcccgtggtcttgtactgcctggatataattatctgggacccttcaacggactcgacaagggagagcccgtcaaccgagcggacgctgttgcgcgagagcacgatatctcgtacaacgagcagctccaggcgggagacaacccctacctcaagtacaaccacgcggacgccgagtttcaggagaagctcgccgacgacacctcctttgggggcaacctcggaaaggcaatctttcaggccaagaagagggttctcgaaccttttggcctggttgaagagcctgttaagacggctccggcaaagaagaggcctgtagagcagtctcctgcggaaccgggcccatcctctggcatcggaaaggcgggcgatcagcctgcgagaaagcgactaaactttggtcagactggagacgccgactccgaacccgacccccagcctctcggagaagcaccagcagccaatcccaatttgggttctgctacaatgtctggaggcggaggcgcgccgatggcagatgatcaacagggtgccgatggagtgggtacttcctcaggaaatcggcattgcgattccacatggctggacaagcgaatcattaccaagtccacccgaacctggagcctgcccacctacaacaaccatctctacaacaagatcatgtccaccaacgccaatggagacagcgcttggtttggcttcagcactccctggggatactttgacttcaaccgcttccactgccacttctcgccccgagactggcaacgactcatcaacaacaactggggcatccggcccaagaaaatgcacgtcaacctcttcaatatccaagtcaaggaagtcacgacgcaagactcgacgacgaccaccaccaataaccttaccagcacgattcaggtgtttgcggacacggagtaccagctgccgtacgtagccagcaacgcccacgagggctgtctcccgccctttccggcggacgtcttcatgcttccgcagtacggctactgcacgctgcaacgcgagaactccaacgatcctgtggcccagagctcgttctactgcctggagtacttccccagccagatgctgaggaccggaaacaactactccatcagctacacgttcgaagacgtgcccttccacagcatgtacatgcacaaccagagcctggaccggctgatgaacccgctcatcgaccagtacttgtggtacctgaataccaccgtatcgaccaacaacacaaacaccttctccaagtcgaccaaaggcgacctggcacgccagaaccgaaactggctgcctggacccgccttccagacggtggccttcaacctgaacggacaaggcaactttggatcgaccaatcactggctttacttgaacaagtacacaatgaatggaaaaagcgtggctatcggccccggccccgtcgtggctcccaagagtacaaccttccaggccgacggcatgatcttcgccaagagcgcagatacggcgacctctgctgcccctcaaaatgaggtcaacatcaccagcgaggcggagacgtccaccgtcaatcccatgatgggcaccaatcctctcattatcaattcgagcaatactccatctacctccgctcccacgatgtcaaatgtatccgttaatcccgtcatgcctggctccgtttggcaagacagagacatctaccttcaaggcccgatctgggccaagattccgcgtaccgacggaaccttccacccctcgcctctgatgggaggattcgggctgcgtcgtcctcctcctcaaatcttcatcaagaacacgcccgttccgtccaatcctccaaccacgttcaatcccgccaagatcaactccttcatcacccaatactcgaccggacaggtcaccgttgagatggaatgggagctcgagaaggaaaacagcaagaggtggaacccggaaatacagttcaccaacaatttccaggtttcggacgggaacatggttcccttctgcgtggactcacgtggtgtctactctgaaccccgccccatcggaacccgctaccttacccgaaccctctaa

SEQ ID NO: 36 - protein, 728 aa, Artificial Sequence
  Synthetic Construct
MSFVDHPPDWLEEVGEGLHEFLELEAGPPKPKPNQQKQDNARGLVLPGYNYLGPFNGLDKGEPVNRADAVAREHDISYNEQLQAGDNPYLKYNHADAEFQEKLADDTSFGGNLGKAIFQAKKRVLEPFGLVEEPVKTAPAKKRPVEQSPAEPGPSSGIGKAGDQPARKRLNFGQTGDADSEPDPQPLGEAPAANPNLGSATMSGGGGAPMADDQQGADGVGTSSGNRHCDSTWLDKRIITKSTRTWSLPTYNNHLYNKIMSTNANGDSAWFGFSTPWGYFDFNRFHCHFSPRDWQRLINNNWGIRPKKMHVNLFNIQVKEVTTQDSTTTTTNNLTSTIQVFADTEYQLPYVASNAHEGCLPPFPADVFMLPQYGYCTLQRENSNDPVAQSSFYCLEYFPSQMLRTGNNYSISYTFEDVPFHSMYMHNQSLDRLMNPLIDQYLWYLNTTVSTNNTNTFSKSTKGDLARQNRNWLPGPAFQTVAFNLNGQGNFGSTNHWLYLNKYTMNGKSVAIGPGPVVAPKSTTFQADGMIFAKSADTATSAAPQNEVNITSEAETSTVNPMMGTNPLIINSSNTPSTSAPTMSNVSVNPVMPGSVWQDRDIYLQGPIWAKIPRTDGTFHPSPLMGGFGLRRPPPQIFIKNTPVPSNPPTTFNPAKINSFITQYSTGQVTVEMEWELEKENSKRWNPEIQFTNNFQVSDGNMVPFCVDSRGVYSEPRPIGTRYLTRTL

SEQ ID NO: 37 - DNA, 2175 bp, Artificial Sequence
  AAV Capsid Ortholog sequence
  CDS (1)..(2175)
atgtcgtttgtcgatcaccccccagactggctcgaggagatcggagaggggctctccgagttcatcggactagaggcgggaccgccgaagcctaagccgggctaccaggaccgcgcgcgcggcctcgtcgtccccggctacaagtacctcgggcccttcaacggactcgacaggggcgagcccgtcaacgcggccgacgcggcggccaagaagcacgacgaggagtacgaccggctcctcaaggcgggggacaacccgtacctcgcctacaaccacgcggacgccgagttccagaaggacctcagcggcgactccagcctagccggcaacgccgcgaacgcgctcttccaggccaagaagagggtcctcgagcccttcggactggtcgagggagagccggagcccaagaagacgccttccgtcaagaggccgcacgcatcgccggactcgtcgagcggcgtcgggaagaagggcgaccagcccgcgcggaagaggctcgactttgggacggagcccgcgggtcaagacggagcgggacgagcggcccaggcagccggagatatggcatctgctgaggtggctgcgggtggtggcggaccagtgggcgacgatgcacaaggtgccgatggagtgggtaatgcctcaggaaattggcattacgattccgtttggatggacggcgctgtcatcaccaagtccacccgaacctggagcctgcccgcctacaacaaccacctctaccgccagatccagtccagcggcaccggagacggcacgtactttggttacagcacgccttggggatacttcgatttcaatcgattccactgccacttttctcccagggactggcagcggctcatcaacaaccactggggcatccgccccaagcggctccactttaagctcttcaacatccaggtcaaggaggtcacgacgaccgacggcaccacgaccatcgccaacaacctcaccagcaccatacaggtctttgcggacacggagtaccagctcccgtacgtgctcggcaacgcccacgagggctgcctgccgccgttcccggcggacgtcttcatgctgccgcagtacgcgtacctgacgctcaacgccaaccccacgaaccagggcgcggccgcgctcagcctgccgcagagcgccttctactgcctggagtactttccgagccagatgctgaggaccgggaacaacttctcgttcagctacgagttcgagaagctgcccttccactcgatgttcatgcacagccagagcctggaccggctcatgaacccgctcatcgaccagtacctctggtacctcaacgccacgaccggcaacaacctctccttcaacaaggccggcgccaagaacttccccgagtacttccgcaactggctgcccgggcccggccagcgcgtgcagcagtggagcacgatcggaacccagaacaacgcgcagaccggcacgtgggccagcgccaacaagtggatcctcatgggcaggtccagcaagatggcccccgggctggcccagccggtcaggaacgcccagaccgtcaccaacggctcgcagctcatcttcaacaacgagaccatcaagggctccaccgcgacggcctccaccgtacactcggggctgctcgtcaccaacgagtcggagaccgccccgaccaaccccaactcggcgacaaagtggggcgtcatgacggacaaccagcagaccacctccactacgccgaccgtcagcgacgacctcgaggcgcacgtcttcccgggcatggtctggcaggaccgcgacatctacctgcagggcccgatatgggccaagatcccggagacggacggccacttccacccgtccccgctcatgggcggcttcgggctcaagaacccgccgccgcagatcctcgtcaagaacacgcccgtgcccgccgtgccgcccacgaccttcacaccgcagaaggtcaactccttcatcacgcagtactccaccggccaggtcaccgtcgagatcgagtgggagctgcgcaaggagaagagcaagcgctggaaccccgagatccagtacacctccaacttcgagaactcggccaacgtacagttctccgtcaacggcgacggcgcgtacatcgagccgcgaccgatcggaacccggtacctcacccacaacctgtaa

SEQ ID NO: 38 - protein, 724 aa, Artificial Sequence
  Synthetic Construct
MSFVDHPPDWLEEIGEGLSEFIGLEAGPPKPKPGYQDRARGLVVPGYKYLGPFNGLDRGEPVNAADAAAKKHDEEYDRLLKAGDNPYLAYNHADAEFQKDLSGDSSLAGNAANALFQAKKRVLEPFGLVEGEPEPKKTPSVKRPHASPDSSSGVGKKGDQPARKRLDFGTEPAGQDGAGRAAQAAGDMASAEVAAGGGGPVGDDAQGADGVGNASGNWHYDSVWMDGAVITKSTRTWSLPAYNNHLYRQIQSSGTGDGTYFGYSTPWGYFDFNRFHCHFSPRDWQRLINNHWGIRPKRLHFKLFNIQVKEVTTTDGTTTIANNLTSTIQVFADTEYQLPYVLGNAHEGCLPPFPADVFMLPQYAYLTLNANPTNQGAAALSLPQSAFYCLEYFPSQMLRTGNNFSFSYEFEKLPFHSMFMHSQSLDRLMNPLIDQYLWYLNATTGNNLSFNKAGAKNFPEYFRNWLPGPGQRVQQWSTIGTQNNAQTGTWASANKWILMGRSSKMAPGLAQPVRNAQTVTNGSQLIFNNETIKGSTATASTVHSGLLVTNESETAPTNPNSATKWGVMTDNQQTTSTTPTVSDDLEAHVFPGMVWQDRDIYLQGPIWAKIPETDGHFHPSPLMGGFGLKNPPPQILVKNTPVPAVPPTTFTPQKVNSFITQYSTGQVTVEIEWELRKEKSKRWNPEIQYTSNFENSANVQFSVNGDGAYIEPRPIGTRYLTHNL

SEQ ID NO: 39 - DNA, 2175 bp, Artificial Sequence
  AAV Capsid Ortholog sequence
  CDS (1)..(2175)
atgtcgtttgtcgatcaccccccagactggctcgaggagatcggagaggggctctccgagtttatcggactagaggcgggaccgccgaagcctaagccgggctaccaggaccgcgcgcgcggcctcgtcgtccccggctacaagtacctcgggcccttcaacggactcgacaggggcgagcccgtcaacgcggccgacgcggcggccaagaagcacgacgaggagtacgaccggctcctcaaggagggggacaacccgtacctcacctacaaccacgccgacgcccagttccagcaggacctccggggcgacgccagcctagccggcaacgccgcgaacgcgctcttccaggccaagaagagggtcctcgagcccttcggactggtcgagggagagccggagccgaagaagacgccttccgtcaagaggccgcacgcatcgccggactcgtcgagcggcgtcgggaagaagggcgaccagcccgcgaggaagaggctcgactttggaacggagcccgcgagtcaagacggagccggacgagcggcgcaggcaaccggagatatggcatctgctgaagtggctgcgggtggtggcggaccagtgggcgacgatgcacaaggtgccgatggagtgggtagcgcctcgggaaattggcattgcgattccgtttggatggacggcgctgtcatcaccaagtccacccgaacctggagcctgcccgcctacaacaaccacctctaccgccagatccagtccagcggcaccggagacggcacgtactttggttacagcacgccttggggatacttcgatttcaatcgattccactgccacttttctcccagggactggcagcggctcatcaacaaccactggggcatccgccccaagcggctccacttcaagctcttcaacatccaggtcaaggaggtcacgacgaccgacggcaccacgaccatcgccaacaacctcaccagcaccatacaggtctttgcggacacggagtaccagctcccgtacgtgctcggcaacgcccacgagggctgcctgccgccgttcccggcggacgtcttcatgctgccgcagtacgcgtacctgacgctcaacgccaacccgacgtcgcagagcgcggccgcgctcagcctgccgcagagcgccttctactgcctcgagtacttcccgagccagatgctgaggaccgggaacaacttctcgttcagctacgagttcgagaagctgcccttccactcgatgttcatgcacagccagagcctggaccggctcatgaacccgctcatcgaccagtacctctggtacctcaacgccacgaccggcaacaacctctccttcaacaaggccggcgccaagaacttccccgagtacttccgcaactggctgcccgggcccggccagcgcgtgcagcagtggagcacgatcgggacccagaacaacgcgcagaccggcacgtgggccagcgccaacaagtggatcctcatgggcaggtccagcaagatggcccccgggctggcccagccggtccggaacgcccagaccgtcaccaacggctcgcagctcatcttcaacaacgagaccatcaagggctccaccgcgacggcctccaccgtgcactcggggctgctcgtcaccaacgagtcggagaccgccccgaccaaccccaactcggcgacaaagtggggcgtcatgacggacaatcagcagaccacctccactacgccgaccgtcagcgacgacctcgaggcgcacgtcttcccgggcatggtctggcaggaccgcgacatctacctgcagggcccgatctgggccaagatcccggagacggacggccacttccacccgtccccgctcatgggcggcttcgggctcaagaacccgccgccgcagatcctcgtcaagaacacgcccgtgcccgccgtgccgcccacgaccttcacgccgcagaaggtcaactccttcatcacgcagtactccaccggccaggtcaccgtcgagatcgagtgggagctgcgcaaggagaagagcaagcgctggaaccccgagatccagtacacgtccaacttcgagaactcggccaacgttcagttctccgtcaacggcgacggcgcgtacatcgagccccgcccgatcggaacccgatacctcacccacaccctgtaa

SEQ ID NO: 40 - protein, 724 aa, Artificial Sequence
  Synthetic Construct
MSFVDHPPDWLEEIGEGLSEFIGLEAGPPKPKPGYQDRARGLVVPGYKYLGPFNGLDRGEPVNAADAAAKKHDEEYDRLLKEGDNPYLTYNHADAQFQQDLRGDASLAGNAANALFQAKKRVLEPFGLVEGEPEPKKTPSVKRPHASPDSSSGVGKKGDQPARKRLDFGTEPASQDGAGRAAQATGDMASAEVAAGGGGPVGDDAQGADGVGSASGNWHCDSVWMDGAVITKSTRTWSLPAYNNHLYRQIQSSGTGDGTYFGYSTPWGYFDFNRFHCHFSPRDWQRLINNHWGIRPKRLHFKLFNIQVKEVTTTDGTTTIANNLTSTIQVFADTEYQLPYVLGNAHEGCLPPFPADVFMLPQYAYLTLNANPTSQSAAALSLPQSAFYCLEYFPSQMLRTGNNFSFSYEFEKLPFHSMFMHSQSLDRLMNPLIDQYLWYLNATTGNNLSFNKAGAKNFPEYFRNWLPGPGQRVQQWSTIGTQNNAQTGTWASANKWILMGRSSKMAPGLAQPVRNAQTVTNGSQLIFNNETIKGSTATASTVHSGLLVTNESETAPTNPNSATKWGVMTDNQQTTSTTPTVSDDLEAHVFPGMVWQDRDIYLQGPIWAKIPETDGHFHPSPLMGGFGLKNPPPQILVKNTPVPAVPPTTFTPQKVNSFITQYSTGQVTVEIEWELRKEKSKRWNPEIQYTSNFENSANVQFSVNGDGAYIEPRPIGTRYLTHTL

SEQ ID NO: 41 - DNA, 2166 bp, Artificial Sequence
  AAV Capsid Ortholog sequence
  CDS (1)..(2166)
atgtcgtttgtcgatcaccccccagactggctcgaggagatcggagaggggctctccgagtttatcggactagaggcgggaccgccgaagcctaagccgggctaccaggaccgcgcgcgcggcctcgtcgtccccggctacaagtacctcgggcccttcaacggactcgacaggggcgagcccgtcaacgcggccgacgcggcggccaagaagcacgacgaggagtacgaccggctcctcaaggcgggggacaacccgtacctcacctacaaccacgcggacgccgagttccagaaggacctccggggcgacgccagcctagccggcaacgccgcgaacgcgctcttccaggccaagaagagggtcctcgagcccttcggactggtcgagggagagccggagccgaagaagacgccttccgtcaagaggccgcacgcatcgccggactcgtcgagcggaatcgggaagaggggcgaccagcccgcgaggaagaggctcgactttggaacggagcccgcgagtcaagacggagcgggaagagcggccaacgcagccggagatatggcatctgctgaggtggctgcgggtggtggcggaccagtgggcgacgatgcacaaggtgccgatggagtgggtcagtcctcgggaaattggcattgcgattccgtttggatgggcgaccgagtgctcaccaagtccacccggacctggagcctgcccacctacaacaaccacctgtacaagcagatcaacggcagcggaaccggggacgcggtgtactttggttacagcacgccttggggatacttcgatttcaatcgattccactgccacttttctccccgagactggcagcgcctcgtcaacaaccactggggcatccgcccccgaaggctcaacttcaagctcttcaacatccaggtcaaggaggtcacgacgacggacggcaccaagaccatcgccaataacctcacgtccaccgtccaggtgtttgcggacacggagcaccagctcccgtacatcctaggctccgcgcacgagggctgcatgccgcccttccccgcggacgtcttcatgctgccgcagtacggctacctcacgctcaacgggcccggcagcaacaacaacaacctcagcacgccctcgagcgccttctactgcctcgagtacttcccgagccagatgctgcgcacgggcaacaacttcgtcttcacctacgagttcgagaaggtgcccttccactcgatgttcatgcacaaccaggcgctggaccgcctcatgaacccgctcgtcgaccagtacctctggtacctcgacgccacctccggcaacaacctcaccttccgcaaggccggcgccaagaacttccccgagtacttccgcaactggatcccaggcccgggctgccgcaaccagcagtggaacaaggtcggcaccaagaacaacccgcagaccggcacgtgggcgtccgccaacaagtggcggctgcagggccgcctcaacaagtacgcgcccggacagcccaacgcccccgcggagggcttcctcaccaacgccggcgacctggccttcgccaacgccaaggccaccggcgccaccacggccgccggaaccgtgcccgccgacatcctgctcaccagcgagagcgagaccaccaccaccaacatgatgtccaacaacggctggggcgcgatcgcctccaacaaccagaacgcgtccgtcgcgcccaccgtgcagtacgaggactcggcgcacgtgctgcccggcatggtctggcaggaccgcgacatctacctgcagggcccgatctgggccaagatcccggagacggacggccacttccacccctcgccgctcatgggcggcttcggactcaagaacccgcccccgcagatcctcatcaagaacacgcccgtgcccgccgaccccccgacgcagttctcctcgcagaagatcaactccttcatcacgcagtactccaccggccagatgaccgtcgagatcgagtgggagctgcgcaaggagaactccaagcgctggaaccccgagatccagtacacggccaacttcaacaacagcgccaacgcgcagttctccgtcaacaacaacggcctctacatcgaggaccgcaccatcggaacccgatacctcacccacaccctgtaa

SEQ ID NO: 42 - protein, 721 aa, Artificial Sequence
  Synthetic Construct
MSFVDHPPDWLEEIGEGLSEFIGLEAGPPKPKPGYQDRARGLVVPGYKYLGPFNGLDRGEPVNAADAAAKKHDEEYDRLLKAGDNPYLTYNHADAEFQKDLRGDASLAGNAANALFQAKKRVLEPFGLVEGEPEPKKTPSVKRPHASPDSSSGIGKRGDQPARKRLDFGTEPASQDGAGRAANAAGDMASAEVAAGGGGPVGDDAQGADGVGQSSGNWHCDSVWMGDRVLTKSTRTWSLPTYNNHLYKQINGSGTGDAVYFGYSTPWGYFDFNRFHCHFSPRDWQRLVNNHWGIRPRRLNFKLFNIQVKEVTTTDGTKTIANNLTSTVQVFADTEHQLPYILGSAHEGCMPPFPADVFMLPQYGYLTLNGPGSNNNNLSTPSSAFYCLEYFPSQMLRTGNNFVFTYEFEKVPFHSMFMHNQALDRLMNPLVDQYLWYLDATSGNNLTFRKAGAKNFPEYFRNWIPGPGCRNQQWNKVGTKNNPQTGTWASANKWRLQGRLNKYAPGQPNAPAEGFLTNAGDLAFANAKATGATTAAGTVPADILLTSESETTTTNMMSNNGWGAIASNNQNASVAPTVQYEDSAHVLPGMVWQDRDIYLQGPIWAKIPETDGHFHPSPLMGGFGLKNPPPQILIKNTPVPADPPTQFSSQKINSFITQYSTGQMTVEIEWELRKENSKRWNPEIQYTANFNNSANAQFSVNNNGLYIEDRTIGTRYLTHTL

SEQ ID NO: 43 - DNA, 2142 bp, Artificial Sequence
  AAV Capsid Ortholog sequence
  CDS (1)..(2142)
atgattcgagtagtcatgtcgtttgttgactatcctccagattggctgaaagaggttgcgggtggactcaaagagttcattgacctcaaaccgggtgcgccgaagcccaaagcgaatcaacaaaaacaagatcaacgcggtcttgtcctcccgggttacaaatacctgggacccttcaacggccttgataaaggccaaccggtgaacaaagccgacgaagtcgcgcgggagcacgacatcgaatacgacaaactcctgaaagaaggagacaacccgtacgtgaagtacaaccacgccgatcaagagtttcaagagaaactcgagggcgacgaatcttttggcggtaacctcggacgagcggtcttccaagccaagaagcggttactggaaccgtttggtttatcggaagagacacctccgagaaagagacccgcttccgagaacaaccagtcagacgtcccggctaagagacgtctgatcctcgatcccgaagacaacggagccgccgaaggagaaccaatcgaacaacaacaaccagccaacgatatggcatctgctgaaatggctgcaggtggtggcgggccagtgggcgacgatgcacagggtgccgatggagtgggtagcgcctcaggaaattggcattgcgattccatttggatgggtgaccgagtcatcaccaaatccactcgcacctggagcctgcccacctacaacaaccacctctacaagcaaatcaactctaacaccaccgatgccaaagaaacctactttggctacagcactccttgggggtattttgacttcaaccggttccactgccacttctcgccgagggactggcaacgactcatcaacaaccattggggcatgcgaccaaagagactcaactttaaactctttaacatccaagtcaaagaggtcacgacgaacgacggtacgaaaacgatcgcgaacaatcttacctcgacggtgcaagtcttcgcggactcagagtaccaactcccctacgtgctaggcaacgcgcacgagggatgctttccaccgtttccggccgacgtcttcatgctaccgcagtatggatacctgaccttgaactcaaacgacaacaaagccatgccacagagctcgttctactgtctggaatattttccgagccagatgctgagaaccggcaacaacttcaccttcagctatgagttcgagagcgttccattccacagcatgtttatgcataaccaatcgctggacagattgatgaatccattgattgaccagtacttatggtacctacactctaccaacagtctatctctaaattttaaaaaagtgaagagagatcatctgtacaaatactacagaaattgggtacccggacccggtcaaagaagacaacaatggagcgaaagtggtggagcaaacaaacaattgacagacttgtggccatacaacaacaaatacaatctgatgggaagatattcttctttggcaccgggaatgccacaagcggcggcagatggattcatcacagccgatggacaattaatattttctaacgtgctacaagaatcagaagaagccagcggtggaaaaccatctccaaatctattaattacaaacgagtcagaagtcagaccaacaaatccagctatcaacgcaccttggggagatgtcgcattaaatcaacaaacgtcaacagtaacaccatcagtagctgaagacaacgaagcccacatttttcctggcatggtctggcaagaccgagacatctacctgcaaggacccatctgggccaagataccagacaccgacggcagcttccacccgtctccgctcatgggtggatttggactcaaacaccctccgccgcagatcctgatcaagaacacgccggtaccgtcggatcctccgctggaattcaacggcaagaagatccagtccttcatcacgcagtactcgaccggtcaggtgacggtggagatcgagtgggagctgaagaaggagcacagcaagcggtggaatccggagatccagtacacctccaacttcgacaacaccgaccgggtgcagttctcggtcaacggggcgggcaactatgtcgaagatcgaccaatcgggacccgttatctcacccacaatctgtaa

SEQ ID NO: 44 - protein, 713 aa, Artificial Sequence
  Synthetic Construct
MIRVVMSFVDYPPDWLKEVAGGLKEFIDLKPGAPKPKANQQKQDQRGLVLPGYKYLGPFNGLDKGQPVNKADEVAREHDIEYDKLLKEGDNPYVKYNHADQEFQEKLEGDESFGGNLGRAVFQAKKRLLEPFGLSEETPPRKRPASENNQSDVPAKRRLILDPEDNGAAEGEPIEQQQPANDMASAEMAAGGGGPVGDDAQGADGVGSASGNWHCDSIWMGDRVITKSTRTWSLPTYNNHLYKQINSNTTDAKETYFGYSTPWGYFDFNRFHCHFSPRDWQRLINNHWGMRPKRLNFKLFNIQVKEVTTNDGTKTIANNLTSTVQVFADSEYQLPYVLGNAHEGCFPPFPADVFMLPQYGYLTLNSNDNKAMPQSSFYCLEYFPSQMLRTGNNFTFSYEFESVPFHSMFMHNQSLDRLMNPLIDQYLWYLHSTNSLSLNFKKVKRDHLYKYYRNWVPGPGQRRQQWSESGGANKQLTDLWPYNNKYNLMGRYSSLAPGMPQAAADGFITADGQLIFSNVLQESEEASGGKPSPNLLITNESEVRPTNPAINAPWGDVALNQQTSTVTPSVAEDNEAHIFPGMVWQDRDIYLQGPIWAKIPDTDGSFHPSPLMGGFGLKHPPPQILIKNTPVPSDPPLEFNGKKIQSFITQYSTGQVTVEIEWELKKEHSKRWNPEIQYTSNFDNTDRVQFSVNGAGNYVEDRPIGTRYLTHNL

SEQ ID NO: 45 - DNA, 2175 bp, Artificial Sequence
  AAV Capsid Ortholog sequence
  CDS (1)..(2175)
atgagtcgagtagtcatgtcgtttgttgatcaccctccagattggctgaagcgtgtggcgagtggcttgactgagttcatcgacctcgaacccggacccccaaaacccaaacccggctatcaggaccagcagcgcggtctcgtggtacccggatacaaatacctcggtccctttaacggtctcgataaaggagaacccgttaacaaggctgacgaggttgctcgagagcacgacctcgagtacaacaagctacttgagcaaggagataatccttatctcaagtacaaccacgccgataaggagtttcaagagaaactctctgacgacacttcctggggcggcaacctcgggaaggcggtttttcaagccaagaagcgtatcctcgagccctttggcctagccgaagagctcggcggtgaaaagacggctcctggaaaaaagcgtcccaggcctccggaacttccaccttcggctccgcattcggacgacgaagaactgattgctcgaccggcagtcagagctcggctcgaaggtcaaaacggagcagcaaacgcaggaccagcagcagcaccagccgcagatatggcatctactgaagtggcttcaggcggtggcggcccaatgggcgacaataaccaaggttccgatggagtgggtagctcctccggaaattggcattgcgattccatttggatggacggcagagtcatcaccaagtctaccagaacctggagcctgcccacctacaacaaccacctctacaagcaaatcaactcaaatgccgaagtcaaagacgaaaagtactttggatatagtactccctggggatactttgatttcaatcgattccactgccacttttctcccagagactggcagcgactcatcaacaaccactggggaatgcgacccaaaagactcaactttaagctgttcaacatccaggtcaaggaggtcacgacgaatgacggaacgacaacgatcgccaataacctcacgtcgaccgtccaggtgtttgcagacacagagtaccagttgccgtacgtgctcgggaatgcgcacgagggctgcctcccgccgttcccggcagacgtgtttatgctgccgcagtacggatacttgactctaaacgacggcaaccaggtcattcctcagagctctttttactgcttggagtactttcccagtcagatgctgagaaccggaaacaactttacgttcagctacgagtttgagaacgtgcctttccacagcatgtttatgcataatcagtctctggaccgactgatgaatcctttagtcgatcagtacctgtggtacctctcggctaccaacggattaaatttacattttagaaaatccaagaagggagcgtttgccagtcaatacagaaactggatgcccggtcctaaccagcgacgtcagcagtggagtaaaggagctggaaacaacaacgccttgaccaatctctgggaatacaaaaacaagttccacatcatgaacagagactcggtactggctccaggactcgcacaggctgcggcggatggatatttcacagccgacggacaattaatttttgctaacgctacacaacaatcggacgaggcttccggtggaaatccaccgagcagcatgttaattaccggtgaatccgaaactcgacccactaatcccggagcaaacaccaagtggggtgaaatggccgacaaccaacagtctacaacggcaaagccgaccatggttatcgacaacgaggctcacatctttcccggcatggtctggcaggatcgggacatttacctgcaagggcccatctgggccaagattccagagacggacggacactttcatccttctccgctgatgggcggattcggtctcaagaatcctcctcctcagatactcatcaagaatacacccgttccggctaatcctccggaagcgttcaccgccaacaagataaactccttcatcacgcagtactcgaccggacaagtaacggtggaaattgaatgggagctcaggaaggagcacagcaagcgctggaacccagaggtgcagtacacgtccaactttgactctacagacagtctgcagtttaccgttaacaacgacggactctacatcgagactcgaccaatcggaacccgtttcctttcccacaatatttaa

SEQ ID NO: 46 - protein, 724 aa, Artificial Sequence
  Synthetic Construct
MSRVVMSFVDHPPDWLKRVASGLTEFIDLEPGPPKPKPGYQDQQRGLVVPGYKYLGPFNGLDKGEPVNKADEVAREHDLEYNKLLEQGDNPYLKYNHADKEFQEKLSDDTSWGGNLGKAVFQAKKRILEPFGLAEELGGEKTAPGKKRPRPPELPPSAPHSDDEELIARPAVRARLEGQNGAANAGPAAAPAADMASTEVASGGGGPMGDNNQGSDGVGSSSGNWHCDSIWMDGRVITKSTRTWSLPTYNNHLYKQINSNAEVKDEKYFGYSTPWGYFDFNRFHCHFSPRDWQRLINNHWGMRPKRLNFKLFNIQVKEVTTNDGTTTIANNLTSTVQVFADTEYQLPYVLGNAHEGCLPPFPADVFMLPQYGYLTLNDGNQVIPQSSFYCLEYFPSQMLRTGNNFTFSYEFENVPFHSMFMHNQSLDRLMNPLVDQYLWYLSATNGLNLHFRKSKKGAFASQYRNWMPGPNQRRQQWSKGAGNNNALTNLWEYKNKFHIMNRDSVLAPGLAQAAADGYFTADGQLIFANATQQSDEASGGNPPSSMLITGESETRPTNPGANTKWGEMADNQQSTTAKPTMVIDNEAHIFPGMVWQDRDIYLQGPIWAKIPETDGHFHPSPLMGGFGLKNPPPQILIKNTPVPANPPEAFTANKINSFITQYSTGQVTVEIEWELRKEHSKRWNPEVQYTSNFDSTDSLQFTVNNDGLYIETRPIGTRFLSHNI

SEQ ID NO: 47 - DNA, 2211 bp, Artificial Sequence
  AAV Capsid Ortholog sequence
  CDS (1)..(2211)
atgagtcgagtagtcatgtcgtttgtcgatcaccctccagattggctgaaagaggccgcgggcggactacgcgagtttctcgatctcgagccgggagcgcctaaacccaaacctaatcaacaaaaacaggaccagcgaggtctggttctgccgggatacaaatatctcggacctttcaacggccttgataaaggccagcccgtcaataaggcggacgaggtcgcgcgagagcacgatctcgagtataacaagctacttgaggcgggcgacaatccctacctcaagtacaaccacgcagaccaagagtttcaggagaaactcgagggggacacttccttcggaggtaacctcggtcgcgcggtattccaggccaagaagagggtgctagagccctttggattggccgaagaagctccggcgaagacggctcctgggaagaaacgtcccaggcccatcgaaccccctccttcctcggagaactcggttgacgaggacttggtcgagcgccaagctcagcgtcaacgtctcggatttcaagacggacccggaaacagcgctaaccaagtccagcaacaaccagccgcaaatatggcatctgctgaagtggctgcgggcagtggcggcccaatgggcgacaatgcacaaggtgccgatggagtgggtagctcctccggaaattggcattgcgattccctttggatgggcgatagggtgatgcgattcccttggatgggagaccgagtcatcaccaagtccacccgaacctggagcctgcccacctacaacaaccatctctacaagcaaatcaattccaactcggatgtcaccgacgaaaagtactttggatacagtaccccgtggggatactttgactttaaccgcttccactgccacttttcaccccgagactggcagcgcctcatcaacaaccattggggtatgcgcccgaagaggctcaactttaagctgttcaacatccaggttaaggaggtcacgacgaacgacggcaccacgaccatcgccaacaacctcacgtctaccgtccagattttcgccgacagcgagtaccagttgccgtacgtgctcgggaatgcgcacgagggctgcctcccgccgttcccggcagacgtgttcatgctgccgcagtacgggtacctcacgttgaacgacggaaatcaagtgattccacaaagttccttctactgtctggagtattttcccagccagatgctgagaaccggcaacaacttttccttcagctacgaatttgaaaacgtgcctttccacagcatgtttatgcacaaccagtctttggatcggttaatgaaccccctcatggaccagtacctgtggtacctgacggccaccaacggcgttcagttgcactttaaaaagtccaagaagggggcgtttgccagccagtttaggaactggctgccgggtcccaaccagcggcgtcagcagtggagcaaaggggccggtaacaacaacgctcagaccaacctgtggagttacaaaaacaagtaccggatcatgaaccgggacagcgtggcggctcctggactggctcaagcggcggccgatggcttcatcacggccgacggacagctcatcttctccaacgccggacaacagtcggatgaagcggctggagctaaaccaccgaccacgttgctgattacagacgagagtgaaactcgtcctaccaacccgggtgccaacaccaaatggggagaagtggcagacaaccaacagacatcttccacggcccccaccatggccatcgacaacgaggcccacatctttcccggaatggtgtggcaggatcgagacatttacctgcagggtcccatctgggccaagattccagagacggacggtcactttcacccgtctcctctgatgggaggattcggactgcgaaatcctcctcctcagattctcatcaaaaacacaccggtaccggccaatccaccagagaccttcaacgccaacaagatcaactcattcatcacccagtactcaaccggacaggttacagtcgaaatagagtgggagctgaagaaggagcacagcaaacgctggaacccggaggtgcagtacacgtccaattttgcctcggcggacagcctccaattcaccgtcaacggagaaggcgtgtacatcgaagacagaccaatcggaacccgctacctctcccacaacatttaa

SEQ ID NO: 48 - protein, 736 aa, Artificial Sequence
  Synthetic Construct
MSRVVMSFVDHPPDWLKEAAGGLREFLDLEPGAPKPKPNQQKQDQRGLVLPGYKYLGPFNGLDKGQPVNKADEVAREHDLEYNKLLEAGDNPYLKYNHADQEFQEKLEGDTSFGGNLGRAVFQAKKRVLEPFGLAEEAPAKTAPGKKRPRPIEPPPSSENSVDEDLVERQAQRQRLGFQDGPGNSANQVQQQPAANMASAEVAAGSGGPMGDNAQGADGVGSSSGNWHCDSLWMGDRVMRFPWMGDRVITKSTRTWSLPTYNNHLYKQINSNSDVTDEKYFGYSTPWGYFDFNRFHCHFSPRDWQRLINNHWGMRPKRLNFKLFNIQVKEVTTNDGTTTIANNLTSTVQIFADSEYQLPYVLGNAHEGCLPPFPADVFMLPQYGYLTLNDGNQVIPQSSFYCLEYFPSQMLRTGNNFSFSYEFENVPFHSMFMHNQSLDRLMNPLMDQYLWYLTATNGVQLHFKKSKKGAFASQFRNWLPGPNQRRQQWSKGAGNNNAQTNLWSYKNKYRIMNRDSVAAPGLAQAAADGFITADGQLIFSNAGQQSDEAAGAKPPTTLLITDESETRPTNPGANTKWGEVADNQQTSSTAPTMAIDNEAHIFPGMVWQDRDIYLQGPIWAKIPETDGHFHPSPLMGGFGLRNPPPQILIKNTPVPANPPETFNANKINSFITQYSTGQVTVEIEWELKKEHSKRWNPEVQYTSNFASADSLQFTVNGEGVYIEDRPIGTRYLSHNI

SEQ ID NO: 49 - DNA, 2175 bp, Artificial Sequence
  AAV5 capsid construct
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 50 - protein, 724 aa, Artificial Sequence
  AAV5 capsid construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 51 - DNA, 3162 bp, Staphylococcus aureus
  CDS (1)..(3162)
atgaaaaggaattatatcttaggattagatatcggaattacatcagtgggttatggaattattgattatgaaactagagatgtcatagatgcgggcgtacgtttatttaaagaggctaatgttgaaaataatgaaggacgacgatcaaaaagaggtgccagaaggcttaagaggcgtcgtagacatagaatacaaagagtaaagaaacttttatttgattacaatttgttgacagatcatagtgagctaagtggaatcaatccttacgaggcgcgcgtaaagggattaagtcaaaaattaagtgaagaggaattttctgcggcattgctacatttagcaaagcgtagaggtgtacataatgttaatgaagtggaagaagatacaggtaatgaattatccactaaagaacaaatttcaagaaatagtaaagcgttagaagagaagtatgttgcagaattacagttggaacgtttgaaaaaagacggtgaagtgagaggttcgattaaccgtttcaaaacatctgactatgtaaaagaagcaaagcagttattaaaagtacaaaaagcatatcatcaacttgatcaatcatttatagacacttatattgatttattggaaacaagaagaacatattatgagggaccaggtgaaggtagcccatttggatggaaagatattaaagaatggtatgaaatgttaatgggacattgtacgtatttcccagaagaattacgtagtgtgaaatatgcctataatgctgatttatataatgcgctgaatgatttgaacaacttggttattacacgagatgagaatgagaagctagagtattatgaaaaattccaaattatcgagaatgtctttaaacaaaagaaaaagccgacgcttaaacaaattgcgaaggaaatcttggtgaatgaagaagacatcaaaggctatcgtgtcacaagtacaggtaaaccagaatttacaaacttgaaagtttatcacgatatcaaagatattacagcaagaaaagaaattatcgagaatgcagagctactcgatcaaatagctaaaatattaactatttaccagtcatcagaagatatacaagaagaattaacaaacctaaattcagaattgacacaagaagagattgaacaaatttcaaacttgaaaggttatacaggaactcataacctttcactaaaggcaataaatttaatattagacgaattgtggcatacgaacgataatcaaatagctattttcaatcgtttgaaacttgtacctaaaaaggtagatttaagccaacaaaaagaaattcctactactttagttgatgattttatactgtctccagtagtgaaacgttcatttatacaatctattaaagttattaacgctattattaaaaaatacggtttgccaaatgatattattattgaacttgcgagagaaaagaattctaaagatgcacaaaaaatgattaatgaaatgcagaagagaaatcgtcaaacgaatgaacgtattgaggaaattataagaacgacaggtaaagaaaatgctaaatatttaattgaaaaaattaagctgcacgatatgcaagaagggaaatgtttatactcgttagaagcaatccctctagaagatttacttaataatccattcaattacgaagtagaccatatcattccacgttctgtttctttcgataactctttcaataataaagtgttggtgaaacaagaagaaaatagtaaaaaaggtaatagaacgccatttcaatatttaagttcttcagattctaaaataagttatgagacattcaaaaagcatattttaaatcttgctaaaggcaaaggtagaatctctaagacgaaaaaagaatatttgttagaagaacgagatatcaatcgcttcagtgtccaaaaagattttattaaccgtaacttagtagatacacgctatgcgacaagagggttaatgaacttattaagatcttattttagagtgaataacttagatgtcaaagtgaaatcgattaatggcggattcacaagtttcttaagaaggaaatggaagttcaaaaaagaaagaaataagggctacaaacaccatgctgaagatgcactgattattgcgaacgctgattttattttcaaagaatggaaaaaactagataaagctaaaaaagtgatggaaaatcaaatgtttgaagaaaagcaagctgaaagtatgcctgaaattgagactgagcaagagtataaagaaatttttataacgcctcatcaaattaaacatattaaggattttaaagattataaatattcacatagagttgataaaaagccgaatagagagttaataaatgatacattatattctacgagaaaagatgacaagggtaatacattaatcgttaataacttaaatggtttatacgataaagataatgataaattgaaaaaattaattaataaatcacctgaaaaattattgatgtatcatcatgatccacaaacatatcaaaaattaaaattgatcatggaacaatatggcgatgagaaaaatccgctttataaatattatgaagaaacaggcaattacttaacaaaatatagtaaaaaagataacggaccagtcatcaaaaaaattaaatattatggtaacaagctaaatgcgcatttagatattacggatgattatccaaatagcagaaataaagtagtaaaactttcattaaaaccatatcgctttgatgtttatttagataatggggtatataaatttgtgacagttaaaaatttagatgttatcaaaaaagaaaactactatgaagttaattcaaagtgttatgaagaagcaaaaaaactgaagaaaattagtaatcaagcagaatttatcgcaagtttttacaataatgacttgattaagattaacggagaattatatagagtcataggtgtaaataatgatctacttaacagaattgaagtaaatatgatagacatcacatatagagaatatttagagaacatgaatgataaaagaccacctagaataattaaaacaatagcaagcaaaacacaatctattaaaaagtattctacagatattctaggcaatctttatgaagtgaagagtaaaaagcatcctcaaatcataaagaaaggatga

SEQ ID NO: 52 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 53 - protein, 984 aa, Campylobacter jejuni
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 54 - protein, 986 aa, Deltaproteobacteria bacterium
MEKRINKIRKKLSADNATKPVSRSGPMKTLLVRVMTDDLKKRLEKRRKKPEVMPQVISNNAANNLRMLLDDYTKMKEAILQVYWQEFKDDHVGLMCKFAQPASKKIDQNKLKPEMDEKGNLTTAGFACSQCGQPLFVYKLEQVSEKGKAYTNYFGRCNVAEHEKLILLAQLKPEKDSDEAVTYSLGKFGQRALDFYSIHVTKESTHPVKPLAQIAGNRYASGPVGKALSDACMGTIASFLSKYQDIIIEHQKVVKGNQKRLESLRELAGKENLEYPSVTLPPQPHTKEGVDAYNEVIARVRMWVNLNLWQKLKLSRDDAKPLLRLKGFPSFPVVERRENEVDWWNTINEVKKLIDAKRDMGRVFWSGVTAEKRNTILEGYNYLPNENDHKKREGSLENPKKPAKRQFGDLLLYLEKKYAGDWGKVFDEAWERIDKKIAGLTSHIEREEARNAEDAQSKAVLTDWLRAKASFVLERLKEMDEKEFYACEIQLQKWYGDLRGNPFAVEAENRVVDISGFSIGSDGHSIQYRNLLAWKYLENGKREFYLLMNYGKKGRIRFTDGTDIKKSGKWQGLLYGGGKAKVIDLTFDPDDEQLIILPLAFGTRQGREFIWNDLLSLETGLIKLANGRVIEKTIYNKKIGRDEPALFVALTFERREVVDPSNIKPVNLIGVDRGENIPAVIALTDPEGCPLPEFKDSSGGPTDILRIGEGYKEKQRAIQAAKEVEQRRAGGYSRKFASKSRNLADDMVRNSARDLFYHAVTHDAVLVFENLSRGFGRQGKRTFMTERQYTKMEDWLTAKLAYEGLTSKTYLSKTLAQYTSKTCSNCGFTITTADYDGMLVRLKKTSDGWATTLNNKELKAEGQITYYNRYKRQTVEKELSAELDRLSEESGNNDISKWTKGRRDEALFLLKKRFSHRPVQEQFVCLDCGHEVHADEQAALNIARSWLFLNSNSTEFKSYKSGKQPFVGAWQAFYKRRLKEVWKPNA

SEQ ID NO: 55 - protein, 757 aa, Biggievirus Mos11
MPKPAVESEFSKVLKKHFPGERFRSSYMKRGGKILAAQGEEAVVAYLQGKSEEEPPNFQPPAKCHVVTKSRDFAEWPIMKASEAIQRYIYALSTTERAACKPGKSSESHAAWFAATGVSNHGYSHVQGLNLIFDHTLGRYDGVLKKVQLRNEKARARLESINASRADEGLPEIKAEEEEVATNETGHLLQPPGINPSFYVYQTISPQAYRPRDEIVLPPEYAGYVRDPNAPIPLGVVRNRCDIQKGCPGYIPEWQREAGTAISPKTGKAVTVPGLSPKKNKRMRRYWRSEKEKAQDALLVTVRIGTDWVVIDVRGLLRNARWRTIAPKDISLNALLDLFTGDPVIDVRRNIVTFTYTLDACGTYARKWTLKGKQTKATLDKLTATQTVALVAIDLGQTNPISAGISRVTQENGALQCEPLDRFTLPDDLLKDISAYRIAWDRNEEELRARSVEALPEAQQAEVRALDGVSKETARTQLCADFGLDPKRLPWDKMSSNTTFISEALLSNSVSRDQVFFTPAPKKGAKKKAPVEVMRKDRTWARAYKPRLSVEAQKLKNEALWALKRTSPEYLKLSRRKEELCRRSINYVIEKTRRRTQCQIVIPVIEDLNVRFFHGSGKRLPGWDNFFTAKKENRWFIQGLHKAFSDLRTHRSFYVFEVRPERTSITCPKCGHCEVGNRDGEAFQCLSCGKTCNADLDVATHNLTQVALTGKTMPKREEPRDAQGTAPARKTKKASKSKAPPAEREDQTPAQEPSQTS

SEQ ID NO: 56 - protein, 1307 aa, Acidaminococcus sp. (strain BV3L6)
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

(19) United States
(12) Patent Application Publication
(10) Pub. No.: US 2022/0364073 A1
(43) Pub. Date: Nov. 17, 2022

(54) ENGINEERING IMMUNE ORTHOGANOL AAV AND IMMUNE STEALTH CRISPR-CAS

(71) Applicant: The Regents of the University of California, Oakland, CA (US)

(72) Inventors: Prashant Mali, La Jolla, CA (US); Nathan Palmer, La Jolla, CA (US); Aditya Kumar, La Jolla, CA (US); Amanda Suhardjo, La Jolla, CA (US)

(21) Appl. No.: 17/851,972

(22) Filed: Jun. 28, 2022

Related U.S. Application Data
(63) Continuation of application No. PCT/US2021/061682, filed on Dec. 2, 2021.
(60) Provisional application No. 63/120,376, filed on Dec. 2, 2020.
(60) Provisional application No. 63/216,135, filed on Jun. 29, 2021.

(51) Int. Cl. C12N 15/10; C12N 15/86; C12N 9/22

(52) U.S. Cl. CPC C12N 15/10; C12N 15/86; C12N 9/22

(57) ABSTRACT

Described herein are methods for engineering proteins and viruses to reduce their immunogenicity, proteins and viruses made by using said methods, including proteins having Cas9 like activity and viruses having AAV5 like activity.

### CROSS REFERENCE TO RELATED APPLICATIONS

This application claims priority under 35 U.S.C. § 119 from Provisional Application Ser. No. 63/120,376, filed Dec. 2, 2020, International Application No. PCT/US2021/061682, filed Dec. 2, 2021, and Provisional Application Ser. No. 63/216,135, filed Jun. 29, 2021, the disclosures of which are incorporated herein by reference in its entirety.

### STATEMENT REGARDING FEDERALLY SPONSORED RESEARCH

This invention was made with Government support under RO1HG009285, RO1CA22826, and RO1GM123313 awarded by the National Institutes of Health. The government has certain rights in the invention.

### TECHNICAL FIELD

Described herein are methods for engineering proteins and viruses to reduce their immunogenicity, proteins and viruses made by using said methods, including proteins having Cas9 like activity and viruses having AAV5 like activity.

### INCORPORATION BY REFERENCE OF SEQUENCE LISTING

Accompanying this filing is a Sequence Listing entitled “Sequence-Listing_ST25.txt”, created on Jun. 27, 2022, and having 491,603 bytes of data, machine formatted on IBM-PC, MS-Windows operating system. The sequence listing is hereby incorporated herein by reference in its entirety for all purposes.

### BACKGROUND

Immunogenicity is a major concern for protein-based therapeutics, particularly those derived from non-human species. Induction of the immune response can render treatments ineffective and cause serious, even life-threatening side effects. One strategy to overcome this issue is to mutate particularly immunogenic epitopes in the therapeutic target. However, this strategy is hindered by the ability of the adaptive immune system to recognize multiple epitopes across large regions of the antigen. While epitope deletion efforts to date have focused on a few major antibody binding sites, it is not possible to make these studies comprehensive due to the vast possible epitope space. Variant library screening has proven to be an effective approach to protein engineering but applying it in this case faces several technical challenges. One problem is the vast mutational space created by the need for full combinatorial libraries. Fully degenerate libraries quickly become intractably large as the number of target sites increases beyond just a few. Narrowing down this space by intelligent selection of library members is necessary to define a reasonable mutational landscape to explore and critical for maximizing the chance of functional hits. Another problem is that reading out combinatorial mutations scattered across large (>1 kb) regions of the protein is extremely difficult using short read sequencing. Using short barcodes attached to each variant to genotype libraries post-screen has proved effective but is limited by the difficulty of constructing large combinatorial libraries in which each member has a short, unique barcode. These issues have generally limited combinatorial library screens to short regions able to be sequenced directly.

### SUMMARY

The disclosure provides a method that overcomes the library size constraints in both the number of unique members and the length of the mutagenized region. The method comprises selecting target regions within a protein of interest using software which predicts HLA-binding and peptide immunogenicity. To generalize immunogenicity predictions and select appropriate targets, an approximation of global HLA allele frequencies is generated using data from the Allele Frequency Net Database. These frequencies were used to scale immunogenicity predictions such that the top hits are the peptides likely to be the most immunogenic epitopes for the largest number of people globally. In order to narrow down the mutational space associated with fully degenerate combinatorial libraries, an approach guided by evolution and natural variation was used. As de-immunizing protein engineering seeks to alter the amino acid sequence of a protein without disrupting functionality, it would be extremely useful to narrow down mutations to those less likely to result in non-functional variants. The method identifies these mutants by leveraging the large amounts of sequencing data available to identify low-frequency SNPs that have been observed in natural environments. Such variants are likely to have limited effect on protein function, as highly deleterious alleles would likely be immediately selected out of the natural population and not appear in sequencing data. Using these more neutral amino acid substitutions in combinatorial libraries increases the likelihood of functional hits with enough epitope variation to evade immune induction. Once the targets are identified and the mutations defined, the library is assembled piecewise using standard synthesis and assembly methods and apply the screen.

In a particular embodiment, the disclosure provides a method for engineering a protein or virus to be less immunoreactive, comprising: identifying target regions of the polynucleotide sequence encoding a protein or virus that are predicted to have human leukocyte antigen (HLA)-binding and/or peptide immunogenicity; identifying single nucleotide polymorphisms (SNPs) or mutations in the targeted region and other regions that are not deleterious to the functioning of the protein; screening a library assembled using standard synthesis and assembly methods by applying the above identifying criteria to find functional variants of the protein or virus; sequencing the functional variants of the protein or virus; mapping genotype to phenotype from the sequences of the functional variants to identify variant candidates that are likely functionally active and have mutations that result in the protein or virus exhibiting less immunogenicity. In another embodiment, the protein is a CRISPR associated protein. In yet another embodiment, the CRISPR associated protein is a Cas9. In a further embodiment, the Cas9 is Streptococcus pyogenes Cas9 (SpCas9). In yet a further embodiment, the target regions are identified using a model that predicts human leukocyte antigen (HLA)-binding and peptide immunogenicity. In a certain embodiment, the prediction model is selected from NetMHC, MHCAttnNet, MHCSeqNET, ACME, NetMHCpan EL 4.1, NetMHCstabpan, SMM, SMMPMBEC, PickPocket, Comblib_Sidney2008, NetMHCcons, MHCflurry 2.0, and IConMHC. In another embodiment, the SNPs or mutations are identified by using phylogenetic methods to scan natural variation among naturally occurring SpCas9, mutations generated in the course of research and engineering efforts, and the Cas9 orthologs of closely related bacterial species. In yet another embodiment, the SNPS or mutations are identified, or further identified, by using immunological prediction of candidate mutations to ensure significant loss of immunogenicity within the targeted region in order to preserve function while reducing immunogenicity. In a further embodiment, the virus is an adeno-associated virus (AAV). In yet a further embodiment, the AAV is selected from AAV1, AAV2, AAV5, AAV6, AAV7, and AAV8. In another embodiment, the AAV is AAV5. In yet another embodiment, the target regions are identified by aligning conserved sequence regions across AAV serotypes. In a further embodiment, the SNPs are identified by aligning the sequences of 50, 60, 70, 80, 90, 100, 110, 120, 130, 140, 150, 160, 170, 180, 190, 200 or more AAV variants that have been sequenced from natural or engineered sources. In yet a further embodiment, the SNPs are located in the target regions. In another embodiment, wherein long-read sequencing technologies capable of sequencing the entire sequence of each protein or viral variant in one sequencing reaction is used to sequence the functional variants of the protein or virus. In yet another embodiment, the long-read sequencing technologies is capable of generating 10-15 Gb of sequencing reads per run. In a further embodiment, a method disclosed herein further comprises the step of evaluating the immunoreactivity of variant candidates in one or more immunoassays. In yet a further embodiment, the one or more immunoassays comprise detecting the presence of antibodies to the variant candidates (AVA antibodies), when the variant candidates are administered in vivo to an animal. In another embodiment, the one or more immunoassays comprise an enzyme-linked immunosorbent assays (ELISAs), electrochemiluminescence (ECL) assays and/or antigen-binding tests, wherein the one or more immunoassays utilize AVA antibodies. The disclosure provides an isolated polypeptide encoded by a polynucleotide sequence having at least 90%, 95%, 97%, 98%, or 99% sequence identity to SEQ ID NO:1, wherein the polypeptide has Cas9 like activity and is less immunogenic that SEQ ID NO:2. In one embodiment, the polynucleotide encodes a polypeptide of SEQ ID NO:2 having one or more mutations selected from the group consisting of: P28L, L237C, Y286Q, S318(H/C), S368C, F498T, L514(T/G), L616G, L623Q, L636D, F704A, L727(P/G), L816D, Y1016(K/G), L1245G, I1273Q, L1282(A/E), and Y1294Q. In particular, the disclosure contemplates any combination of the foregoing mutations to SEQ ID NO:2, wherein the number of mutations comprises 1, 2, 3, 4, 5, 6, 7, 8, 9, 10, 11, 12, 13, 14, 15, 16, 17, or 18 mutations as recited above. In particular, the disclosure contemplates any combination of the foregoing mutations to SEQ ID NO:2, wherein the number of mutations comprises 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, or 18 mutations as recited above. In addition, the polypeptide may have 1-10 additional conservative mutations at positions other than those set forth above.

The disclosure also provides an isolated polypeptide having a sequence that has at least 90%, 95%, 97%, 98%, or 99% sequence identity to the sequence presented in SEQ ID NO:2 wherein the protein has Cas9 like activity and is immuno-silenced compared to a polypeptide of SEQ ID NO:2. In one embodiment, the polypeptide comprises the sequence of SEQ ID NO:2 and having one or more mutations selected from the group consisting of: P28L, L237C, Y286Q, S318(H/C), S368C, F498T, L514(T/G), L616G, L623Q, L636D, F704A, L727(P/G), L816D, Y1016(K/G), L1245G, I1273Q, L1282(A/E), and Y1294Q. In particular, the disclosure contemplates any combination of the foregoing mutations to SEQ ID NO:2, wherein the number of mutations comprises 1, 2, 3, 4, 5, 6, 7, 8, 9, 10, 11, 12, 13, 14, 15, 16, 17, or 18 mutations as recited above. In particular, the disclosure contemplates any combination of the foregoing mutations to SEQ ID NO:2, wherein the number of mutations comprises 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, or 18 mutations as recited above. In addition, the polypeptide may have 1-10 additional conservative mutations at positions other than those set forth above.

The disclosure also provides a virus comprising a viral capsid encoded by a polynucleotide having 90-99% sequence identity to SEQ ID NO:49 and encoding a viral capsid polypeptide having AAV5 like activity and is immuno-silenced compared to a wild-type viral capsid of SEQ ID NO:50. In one embodiment, the viral capsid comprises a sequence of SEQ ID NO:50 and comprises two or more mutation selected from the group consisting of R42G, P47L, Y49C, G55S, N56H, G57S, D59Y, Y89H, L90(P/I), A95V, D96G, E98(K/Q), F99L, T107A, S108P, Q119(R/E), R123(L/T), V124A, V131A, E132(G/R), E133(Q/D), G134(V/S), T137A, A214V, S222T, T223(A/K), S267A, Y272H, F273L, W287R, L290P, I291V, I309V, K312(E/R), N400D, F402L, F413L, S415T, 5416(M/G), Q604R, P606Q, I607T, F627L, L629(P/F), K630E, H631(R/N), S663G, T664A, R682C, W683R, N684(D/S), T717(S/A), Y719F, and L720P. In particular, the disclosure contemplates any combination of the foregoing mutations to SEQ ID NO:50, wherein the number of mutations comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54 mutations as recited above. In particular, the disclosure contemplates any combination of the foregoing mutations to SEQ ID NO:50, wherein the number of mutations comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, or 54 mutations as recited above. In a particular embodiment, the viral capsid comprises no more than 60 mutations. In addition, the viral capsid may have 1-10 additional conservative mutations at positions other than those set forth above.

In a certain embodiment, the disclosure also provides for an AAV system comprising the virus disclosed herein that has AAV5 like activity. In a further embodiment, the AAV system is used for gene therapy.

### DESCRIPTION OF DRAWINGS

FIG. 1A-B provides (A) schematic of library design methodology; (B) tables showing the location, codon information, etc. for the generation of a library of hCas9 constructs using a method disclosed herein. As indicated in the lower table, there were 56 total constructs generated from a library having the size of 995328.

FIG. 2 provides a library construction schematic.

FIG. 3 provides Immunogenicity scores of Cas orthologs. As more immunogenic epitopes are mutated total protein immunogenicity decreases rapidly at first and then with diminishing returns at high mutation numbers.

FIG. 4 provides Cas9 functional screen schematic.

FIG. 5 provides mutation density distribution of Cas9 library before and after screening.

FIG. 6 provides HeLa cells transduced with Cas9 and HPRT1 or non-targeting guide were treated with 0-14 μg/mL 6TG. Viable cells are shown by crystal violet staining.

FIG. 7 provides replicate correlation of post-screen element frequencies on logarithmic (left) and linear (right) scale. R2=0.925.

FIG. 8 provides pre- and post-screen allele frequencies at each of the 18 mutation sites. Each site shows enrichment of the wild-type allele, but most sites retain a substantial fraction of mutant alleles.

FIG. 9 provides neighbor score as a metric for differentiating true positive hits. Left: Neighbor score is positively correlated with screen score. Right: Divergence between neighbor score and screen score decreases as read coverage increases.

FIG. 10 shows a network diagram of screen hits. Nodes are Cas9 variant hits with high coverage in each replicate and enrichment greater than wild-type. Edges between nodes correspond to the distance between genotypes and are weighted as 1/n for n<5 where n is the number of non-shared mutations. Node colors correspond to mutational distance from wild-type. Dark blue=1, blue=2, light blue=3, white=4, pink=>4, red=tested variant, yellow=variant testing underway. Network layout was computed using the Fruchterman-Reingold algorithm.

FIG. 11 shows HDR efficiencies for wildtype and variant Cas as quantified by FACS.

FIG. 12 provides NHEJ efficiencies for wildtype and variant Cas as quantified by NGS.

FIG. 13 provides Correlation between pLDDT and epistasis in our Cas9 screen (top left), as well as third party double mutant datasets for GFP (top right, DOI: 10.1038/nature17995), human YAP65 WW domain (bottom left, DOI: 10.1073/pnas.1209751109), and yeast Pab1 RRM domain (bottom right, DOI: 10.1261/rna.040709.113).

FIG. 14 shows maximum likelihood cladogram of AAVs including the main human serotypes and new tested orthologs.

FIG. 15 provides Viral formation titers of AAV orthologs relative to AAV5.

FIG. 16 provides tables showing the location, codon information, etc. for the generation of a library of AAV5 constructs using a method disclosed herein. As indicated in the lower table, there were 40 total constructs generated from a library having the size of 2097152.

FIG. 17 provides a DNA sequence (SEQ ID NO:49) listing an exemplary AAV5 construct that has modified so as to be less immunoreactive.

FIG. 18 provides an AA sequence (SEQ ID NO:50) of an exemplary AAV5 construct that has modified so as to be less immunoreactive.

### DETAILED DESCRIPTION

As used herein and in the appended claims, the singular forms “a,” “an,” and “the” include plural referents unless the context clearly dictates otherwise. Thus, for example, reference to “a cell” includes a plurality of such cells and reference to “the fragment” includes reference to one or more fragments and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this disclosure is not limited to the particular methodology, protocols, and reagents, etc., described herein and as such may vary. The terminology used herein is for the purpose of describing particular embodiments or aspects only and is not intended to limit the scope of the present disclosure.

Other than in the operating examples, or where otherwise indicated, all numbers expressing quantities of ingredients or reaction conditions used herein should be understood as modified in all instances by the term “about.” The term “about” when used to described the present invention, in connection with percentages means±1%. The term “about,” as used herein can mean within an acceptable error range for the particular value as determined by one of ordinary skill in the art, which can depend in part on how the value is measured or determined, e.g., the limitations of the measurement system. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value can be assumed. Also, where ranges and/or subranges of values are provided, the ranges and/or subranges can include the endpoints of the ranges and/or subranges. In some cases, variations can include an amount or concentration of 20%, 10%, 5%, 1%, 0.5%, or even 0.1% of the specified amount.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “adeno-associated virus” or “AAV” as used herein refers to a member of the class of viruses associated with this name and belonging to the genus dependoparvovirus, family Parvoviridae. Multiple serotypes of this virus are known to be suitable for gene delivery; all known serotypes can infect cells from various tissue types. Non-limiting exemplary serotypes useful in the methods disclosed herein include any of the 11 or 12 serotypes, e.g., AAV2, AAV5, and AAV8, or variant serotypes such as AAV-DJ. The AAV structural particle is composed of 60 protein molecules made up of VP1, VP2 and VP3. Each particle contains approximately 5 VP1 proteins, 5 VP2 proteins and 50 VP3 proteins ordered into an icosahedral structure. Non-limiting exemplary VP1 sequences useful in the methods disclosed herein are provided below.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and 0-phosphoserine. In some embodiments, an amino acid analog refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. In some embodiments, an amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature. In certain instances one or more D-amino acids can be used in various peptide compositions of the disclosure. The disclosure provides various peptides that are useful for treating various diseases and infections. These peptides can comprise naturally occurring amino acid. In other embodiments, the peptides can comprise non-natural amino acids. The use of non-natural amino acids can improve the peptides stability, decrease degradation and/or improve biological activity. For example, in some embodiments, one or more D-amino acids. In other embodiments, retroinverso peptides are contemplated using various amino acid configurations.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “Cas9” refers to a CRISPR-associated, RNA-guided endonuclease such as Streptococcus pyogenes Cas9 (spCas9; see Accession Number Q99ZW2.1, the sequence of which is incorporated herein by reference) and orthologs and biological equivalents thereof. Biological equivalents of Cas9 include, but are not limited to, C2c1 from Alicyclobacillus acideterrestris and Cpf1 (which performs cutting/cleaving functions analogous to Cas9) from various bacterial species including Acidaminococcus spp. and Francisella novicida U112. Cas9 may refer to an endonuclease that causes double stranded breaks in DNA, a nickase variant such as a RuvC or HNH mutant that causes a single stranded break in DNA, as well as other variations such as deadCas-9 (“dCas9”), which lack endonuclease activity. Cas9 may also refer to “split-Cas9” in which Cas9 is split into two halves—C-terminal Cas9 (C-Cas9) and an N-terminal Cas-9 (N-Cas9)—which can be fused with two intein moieties. See, e.g., U.S. Pat. No. 9,074,199 B1; Zetsche et al. (2015) Nat Biotechnol. 33(2):139-42; Wright et al. (2015) PNAS 112(10) 2984-89. Non-limiting examples of commercially available sources of SpCas9 comprising plasmids can be found under the following AddGene reference numbers:

42230: PX330; SpCas9 and single guide RNA;

48138: PX458; SpCas9-2A-EGFP and single guide RNA;

62988: PX459; SpCas9-2A-Puro and single guide RNA;

48873: PX460; SpCas9n (D10A nickase) and single guide RNA;

48140: PX461; SpCas9n-2A-EGFP (D10A nickase) and single guide RNA;

62987: PX462; SpCas9n-2A-Puro (D10A nickase) and single guide RNA; and

48137: PX165; SpCas9;

all of which are incorporated herein by reference.

As used herein, the term “CRISPR” refers to Clustered Regularly Interspaced Short Palindromic Repeats (CRISPR). CRISPR may also refer to a technique or system of sequence-specific genetic manipulation relying on the CRISPR pathway. A CRISPR recombinant expression system can be programmed to cleave a target polynucleotide using a CRISPR endonuclease and a guideRNA. A CRISPR system can be used to cause double stranded or single stranded breaks in a target polynucleotide. A CRISPR system can also be used to recruit proteins or label a target polynucleotide. In some aspects, CRISPR-mediated gene editing utilizes the pathways of nonhomologous end-joining (NHEJ) or homologous recombination to perform the edits. These applications of CRISPR technology are known and widely practiced in the art. See, e.g., U.S. Pat. No. 8,697,359 and Hsu et al. (2014) Cell 156(6): 1262-1278.

As used herein, the term “domain” can refer to a particular region of a larger molecule (e.g., a particular region of a protein or polypeptide), which can be associated with a particular function. For example, “a domain which binds to a cognate” can refer to the domain of a protein that binds one or more receptors or other protein moieties. Similarly, a corresponding coding sequence for a particular polypeptide domain can be referred to as a polynucleotide domain.

The term “encode” as it is applied to polynucleotides can refer to a polynucleotide which is said to “encode” a polypeptide if, in its native state or when manipulated by methods well known to those skilled in the art, it can be transcribed and/or translated to produce the mRNA for the polypeptide and/or a fragment thereof. In some cases the antisense strand is the complement of such a nucleic acid, and the encoding sequence can be deduced therefrom.

The terms “equivalent” or “biological equivalent” are used interchangeably when referring to a particular molecule, biological, or cellular material and intend those having minimal homology while still maintaining desired structure or functionality.

As used herein, “expression” can refer to the process by which polynucleotides are transcribed into mRNA and/or the process by which the transcribed mRNA is subsequently being translated into peptides, polypeptides, or proteins. If the polynucleotide is derived from genomic DNA, expression can include splicing of the mRNA in a eukaryotic cell.

As used herein, the term “functional” may be used to modify any molecule, biological, or cellular material to intend that it accomplishes a particular, specified effect.

The term “gRNA” or “guide RNA” as used herein refers to the guide RNA sequences used to target specific genes for correction employing the CRISPR technique. Techniques of designing gRNAs and donor therapeutic polynucleotides for target specificity are well known in the art. For example, Doench, J., et al. Nature biotechnology 2014; 32(12): 1262-7, Mohr, S. et al. (2016) FEBS Journal 283: 3232-38, and Graham, D., et al. Genome Biol. 2015; 16: 260. gRNA comprises or alternatively consists essentially of, or yet further consists of a fusion polynucleotide comprising CRISPR RNA (crRNA) and trans-activating CRIPSPR RNA (tracrRNA); or a polynucleotide comprising CRISPR RNA (crRNA) and trans-activating CRIPSPR RNA (tracrRNA). In some aspects, a gRNA is synthetic (Kelley, M. et al. (201) J of Biotechnology 233 (2016) 74-83).

“Homology” or “identity” or “similarity” can refer to sequence similarity between two peptides or between two nucleic acid molecules. Homology can be determined by comparing a position in each sequence which can be aligned for purposes of comparison. For example, when a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the disclosure.

Homology refers to a percent (%) identity of a sequence to a reference sequence. As a practical matter, any particular sequence can be at least 50%, 60%, 70%, 80%, 85%, 90%, 92%, 95%, 96%, 97%, 98% or 99% identical to any sequence described herein. Whether such particular peptide, polypeptide or nucleic acid sequence has a particular identity/homology can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence, the parameters can be set such that the percentage of identity is calculated over the full length of the reference sequence and that gaps in homology of up to 5% of the total reference sequence are allowed.

For example, in a specific embodiment the identity between a reference sequence (query sequence, i.e., a sequence of the disclosure) and a subject sequence, also referred to as a global sequence alignment, can be determined using the FASTDB computer program based on the algorithm of Brutlag et al. (Comp. App. Biosci. 6:237-245 (1990)). In some cases, parameters for a particular embodiment in which identity is narrowly construed, used in a FASTDB amino acid alignment, can include: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject sequence, whichever is shorter. According to this embodiment, if the subject sequence is shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that are lateral to the N- and C-terminal of the subject sequence, which are not matched/aligned with a corresponding subject residue, as a percent of the total bases of the query sequence. A determination of whether a residue is matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some cases, only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence are considered for this manual correction. For example, a 90 residue subject sequence can be aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity can be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for.

“Hybridization” can refer to a reaction in which one or more polynucleotides react to form a complex that is stabilized via hydrogen bonding between the bases of the nucleotide residues. The hydrogen bonding can occur by Watson-Crick base pairing, Hoogstein binding, or in any other sequence-specific manner. The complex can comprise two strands forming a duplex structure, three or more strands forming a multi-stranded complex, a single self-hybridizing strand, or any combination of these. A hybridization reaction can constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

As used herein, the term “immune orthogonal” refers to a lack of immune cross-reactivity between two or more antigens. In some embodiments, the antigens are proteins (e.g., Cas9). In some embodiments, the antigens are viral antigens associated with a particular viral vector (e.g., AAV). As is recognized in the art, antigens typically include antigenic determinants having a particular sequence of 3 dimensional structure. Moreover, an antigenic determinant can comprise a domain or subsequence of a larger polypeptide or molecular sequence. In some embodiments, antigens that are immune orthogonal do not share an amino acid sequence of greater than 5, greater than 6, greater than 7, greater than 8, greater than 9, greater than 10, greater than 11, greater than 12, greater than 13, greater than 14, greater than 15, or greater than 16 consecutive amino acids. In some embodiments, antigens that are immune orthogonal do not share any highly immunogenic peptides. In some embodiments, antigens that are immune orthogonal do not share affinity for a major histocompatibility complex (e.g., MHC class I or class II). Antigens that are immune orthogonal are amenable for sequential dosing to evade a host immune system.

The term “immunosilent” refers to an epitope or foreign peptide, polypeptide or protein that does not elicit an immune response from a host upon administration. In some embodiments, the peptide, polypeptide or protein does not elicit an adaptive immune response. In some embodiments, the peptide, polypeptide or protein does not elicit an innate immune response. In some embodiments, the peptide, polypeptide or protein does not elicit either an adaptive or an innate immune response. In some embodiments, an immunosilent peptide, polypeptide or protein has reduced immunogenicity.

The term “isolated” as used herein can refer to molecules or biologicals or cellular materials being substantially free from other materials. In one aspect, the term “isolated” can refer to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also can refer to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and may not be found in the natural state. In some cases, the term “isolated” is also used herein to refer to polypeptides which are isolated from other cellular proteins and is meant to encompass both purified and recombinant polypeptides. In some cases, the term “isolated” is also used herein to refer to cells or tissues that are isolated from other cells or tissues and is meant to encompass both cultured and engineered cells or tissues.

“Messenger RNA” or “mRNA” is a nucleic acid molecule that is transcribed from DNA and then processed to remove non-coding sections known as introns. In some cases, the resulting mRNA is exported from the nucleus (or another locus where the DNA is present) and translated into a protein. The term “pre-mRNA” can refer to the strand prior to processing to remove non-coding sections. mRNA has “U” in place of “T” in cDNA coding sequences.

The term “Major Histocompatibility Complex” (MHC) refers to a family of proteins responsible for the presentation of peptides, including self and non-self (antigenic) to T-cells. T-cells recognize antigenic peptides and trigger a cascade of events which leads to the destruction of pathogens and infected cells. The MHC family is divided into three subgroups: class I, class II, and class III. Class I MHC molecules have β2 subunits that are only recognized by CD8 co-receptors. Class II MHC molecules have β1 and β2 subunits that are only recognized by CD4 co-receptors. In this way MHC molecules chaperone which type of lymphocytes may bind to the given antigen with high affinity, since different lymphocytes express different T-Cell Receptor (TCR) co-receptors. In general, MHC class I molecules bind short peptides, whose N- and C-terminal ends are anchored into pockets located at the ends of a peptide binding groove. While the majority of the peptides are nine amino acid residues in length, longer peptides can be accommodated by the bulging of their central portion, resulting in binding peptides of length 8 to 15. Peptides binding to class II proteins are not constrained in size and can vary from 11 to 30 amino acids long. The peptide binding groove in the MHC class II molecules is open at both ends, which enables binding of peptides with relatively longer length. The “core” refers to the amino acid residues that contribute the most to the recognition of the peptide. In some embodiments, the core is nine amino acids in length. In addition to the core, the flanking regions are also important for the specificity of the peptide to the MHC molecule.

The term “ortholog” is used in reference of another gene or protein and intends a homolog of said gene or protein that evolved from the same ancestral source or which are evolved artificially using molecular biology and genetic engineering. Orthologs may or may not retain the same function as the gene or protein to which they are orthologous. Non-limiting examples of Cas9 orthologs include S. aureus Cas9 (“spCas9”), S. thermophiles Cas9, L. pneumophilia Cas9, N. lactamica Cas9, N. meningitides Cas9, B. longum Cas9, A. muciniphila Cas9, and O. laneus Cas9.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. Non-limiting exemplary promoters include CMV promoter and U6 promoter.

The term “protein”, “peptide” and “polypeptide” are used interchangeably and in their broadest sense to refer to a compound of two or more subunit amino acids, amino acid analogs or peptidomimetics. The subunits can be linked by peptide bonds. In another embodiment, the subunit can be linked by other bonds, e.g., ester, ether, etc. A protein or peptide can contain at least two amino acids and no limitation is placed on the maximum number of amino acids which can comprise a protein's or peptide's sequence. As mentioned above, the term “amino acid” can refer to either natural and/or unnatural or synthetic amino acids, including glycine and both the D and L optical isomers, amino acid analogs and peptidomimetics. As used herein, the term “fusion protein” can refer to a protein comprised of domains from more than one naturally occurring or recombinantly produced protein, where generally each domain serves a different function. In this regard, the term “linker” can refer to a peptide fragment that is used to link these domains together—optionally to preserve the conformation of the fused protein domains and/or prevent unfavorable interactions between the fused protein domains which can compromise their respective functions.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and can perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also can refer to both double and single stranded molecules. Unless otherwise specified or required, any embodiment of this disclosure that is a polynucleotide can encompass both the double stranded form and each of two complementary single stranded forms known or predicted to make up the double stranded form.

The term “polynucleotide sequence” can be the alphabetical representation of a polynucleotide molecule. This alphabetical representation can be input into databases in a computer having a central processing unit and used for bioinformatics applications such as functional genomics and homology searching.

Similarly, the term “polypeptide sequence”, “peptide sequence” or “protein sequence” can be the alphabetical representation of a polypeptide molecule. This alphabetical representation can be input into databases in a computer having a central processing unit and used for bioinformatics applications such as functional proteomics and homology searching.

As used herein, the term “recombinant expression system” refers to a genetic construct or constructs for the expression of certain genetic material formed by recombination.

As used herein, the term “recombinant protein” can refer to a polypeptide or peptide which is produced by recombinant DNA techniques, wherein generally, DNA encoding the polypeptide or peptide is inserted into a suitable expression vector which is in turn used to transform a host cell to produce the heterologous polypeptide or peptide.

The term “sequencing” as used herein, can comprise bisulfite-free sequencing, bisulfite sequencing, TET-assisted bisulfite (TAB) sequencing, ACE-sequencing, high-throughput sequencing, Maxam-Gilbert sequencing, massively parallel signature sequencing, Polony sequencing, 454 pyrosequencing, Sanger sequencing, Illumina sequencing, SOLiD sequencing, Ion Torrent semiconductor sequencing, DNA nanoball sequencing, Heliscope single molecule sequencing, single molecule real time (SMRT) sequencing, nanopore sequencing, shot gun sequencing, RNA sequencing, Enigma sequencing, or any combination thereof.

As used herein, the term “subject” is intended to mean any animal. In some embodiments, the subject may be a mammal; in further embodiments, the subject may be a bovine, equine, feline, murine, porcine, canine, human, or rat.

As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection (e.g., using commercially available reagents such as, for example, LIPOFECTIN® (Invitrogen Corp., San Diego, Calif.), LIPOFECTAMINE® (Invitrogen), FUGENE® (Roche Applied Science, Basel, Switzerland), JETPEI™ (Polyplus-transfection Inc., New York, N.Y.), EFFECTENE® (Qiagen, Valencia, Calif.), DREAMFECT™ (OZ Biosciences, France) and the like), or electroporation. Suitable methods for transforming or transfecting host cells can be found in Sambrook, et al. (Molecular Cloning: A Laboratory Manual. 2nd, ed., Cold Spring harbor Laboratory, Cold Spring Harbor Laboratory Press, Cold Spring Harbor, N.Y., 1989), and other laboratory manuals. Standard recombinant DNA and molecular cloning techniques used herein are well known in the art and are described in Sambrook, J., Fritsch, E. F. and Maniatis, T., Molecular Cloning: A Laboratory Manual, 2^{nd} ed.; Cold Spring Harbor Laboratory: Cold Spring Harbor, N.Y., (1989) and by Silhavy, T. J., Bennan, M. L. and Enquist, L. W., Experiments with Gene Fusions; Cold Spring Harbor Laboratory: Cold Spring Harbor, N.Y., (1984); and by Ausubel, F. M. et. al., Current Protocols in Molecular Biology, Greene Publishing and Wiley-Interscience (1987) each of which are hereby incorporated by reference in its entirety. Additional useful methods are described in manuals including Advanced Bacterial Genetics (Davis, Roth and Botstein, Cold Spring Harbor Laboratory, 1980), Experiments with Gene Fusions (Silhavy, Berman and Enquist, Cold Spring Harbor Laboratory, 1984), Experiments in Molecular Genetics (Miller, Cold Spring Harbor Laboratory, 1972) Experimental Techniques in Bacterial Genetics (Maloy, in Jones and Bartlett, 1990), and A Short Course in Bacterial Genetics (Miller, Cold Spring Harbor Laboratory 1992) each of which are hereby incorporated by reference in its entirety.

The terms “treat”, “treating” and “treatment”, as used herein, refers to ameliorating symptoms associated with a disease or disorder (e.g., cancer, Covid-19 etc.), including preventing or delaying the onset of the disease or disorder symptoms, and/or lessening the severity or frequency of symptoms of the disease or disorder.

As used herein, the term “vector” can refer to a nucleic acid construct deigned for transfer between different hosts, including but not limited to a plasmid, a virus, a cosmid, a phage, a BAC, a YAC, etc. In some embodiments, a “viral vector” is defined as a recombinantly produced virus or viral particle that comprises a polynucleotide to be delivered into a host cell, either in vivo, ex vivo or in vitro. In some embodiments, plasmid vectors can be prepared from commercially available vectors. In other embodiments, viral vectors can be produced from baculoviruses, retroviruses, adenoviruses, AAVs, etc. according to techniques known in the art. In one embodiment, the viral vector is a lentiviral vector. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. In aspects where gene transfer is mediated by a retroviral vector, a vector construct can refer to the polynucleotide comprising the retroviral genome or part thereof, and a gene of interest. Further details as to modern methods of vectors for use in gene transfer can be found in, for example, Kotterman et al. (2015) Viral Vectors for Gene Therapy: Translational and Clinical Outlook Annual Review of Biomedical Engineering 17. Vectors that contain both a promoter and a cloning site into which a polynucleotide can be operatively linked are well known in the art. Such vectors are capable of transcribing RNA in vitro or in vivo and are commercially available from sources such as Agilent Technologies (Santa Clara, Calif.) and Promega Biotech (Madison, Wis.). In one aspect, the promoter is a pol III promoter.

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and ‘Vector” can be used interchangeably. However, the disclosure is intended to include such other forms of expression vectors, such as viral vectors (e.g., replication defective retroviruses, adenoviruses and adeno-associated viruses), which serve equivalent functions. Typically, the vector or plasmid contains sequences directing transcription and translation of a relevant gene or genes, a selectable marker, and sequences allowing autonomous replication or chromosomal integration. Suitable vectors comprise a region 5′ of the gene which harbors transcriptional initiation controls and a region 3′ of the DNA fragment which controls transcription termination. Both control regions may be derived from genes homologous to the transformed host cell, although it is to be understood that such control regions may also be derived from genes that are not native to the species chosen as a production host.

Typically, the vector or plasmid contains sequences directing transcription and translation of a gene fragment, a selectable marker, and sequences allowing autonomous replication or chromosomal integration. Suitable vectors comprise a region 5′ of the gene which harbors transcriptional initiation controls and a region 3′ of the DNA fragment which controls transcription termination. Both control regions may be derived from genes homologous to the transformed host cell, although it is to be understood that such control regions may also be derived from genes that are not native to the species chosen as a production host.

Initiation control regions or promoters, which are useful to drive expression of the relevant pathway coding regions in the desired host cell are numerous and familiar to those skilled in the art. Virtually any promoter capable of driving these genetic elements is suitable for the present invention including, but not limited to, lac, ara, tet, trp, IPL, IPR, T7, tac, and trc (useful for expression in Escherichia coli and Pseudomonas); the amy, apr, npr promoters and various phage promoters useful for expression in Bacillus subtilis, and Bacillus licheniformis; nisA (useful for expression in gram positive bacteria, Eichenbaum et al. Appl. Environ. Microbiol. 64(8):2763-2769 (1998)); and the synthetic P11 promoter (useful for expression in Lactobacillus plantarum, Rud et al., Microbiology 152:1011-1019 (2006)). Termination control regions may also be derived from various genes native to the preferred hosts.

Immunogenicity is a major concern for protein-based therapeutics, particularly those derived from non-human species. Induction of the immune response can render treatments ineffective and cause serious, even life-threatening side effects. One strategy to overcome this issue is to mutate particularly immunogenic epitopes in the therapeutic target. However, this strategy is hindered by the ability of the adaptive immune system to recognize multiple epitopes across large regions of the antigen. While epitope deletion efforts to date have focused on a few major antibody binding sites, it is not possible to make these studies comprehensive due to the vast possible epitope space. Variant library screening has proven to be an effective approach to protein engineering but applying it in this case faces several technical challenges. One problem is the vast mutational space created by the need for full combinatorial libraries. Fully degenerate libraries quickly become intractably large as the number of target sites increases beyond just a few. Narrowing down this space by intelligent selection of library members is necessary to define a reasonable mutational landscape to explore and critical for maximizing the chance of functional hits. Another problem is that reading out combinatorial mutations scattered across large (>1 kb) regions of the protein is extremely difficult using short read sequencing. Using short barcodes attached to each variant to genotype libraries post-screen has proved effective but is limited by the difficulty of constructing large combinatorial libraries in which each member has a short, unique barcode. These issues have generally limited combinatorial library screens to short regions able to be sequenced directly.

The scale of engineering which would be required to generate an effectively de-immunized Cas9, for example, is not fully understood, as combinatorial de-immunization efforts at the scale of proteins thousands of amino acids long have not yet been possible. Therefore, to roughly estimate these parameters an immunogenicity scoring metric was developed that takes into account all epitopes across a protein and the known diversity of MHC variants in a species weighted by population frequency to generate a single combined score representing the average immunogenicity of a full-length protein as a function of each of its immunogenic epitopes. Formally, this score is calculated as:

$${I}_{x} = \frac{{\sum }_{i}^{m} {\sum }_{j}^{n} {w}_{j} * ( 1 - log log ( {k}_{ij} * \hat{J} ) )}{y}$$

where Iₓ=Immunogenicity score of protein x, i=epitopes, j=HLA alleles, Ĵ=allele specific standardization coefficient, wⱼ=HLA allele weights, kᵢⱼ=predicted binding affinity of epitope i to allele j, pᵢⱼ=percentile rank of epitope i binding to allele j, and y=protein specific scaling factor.

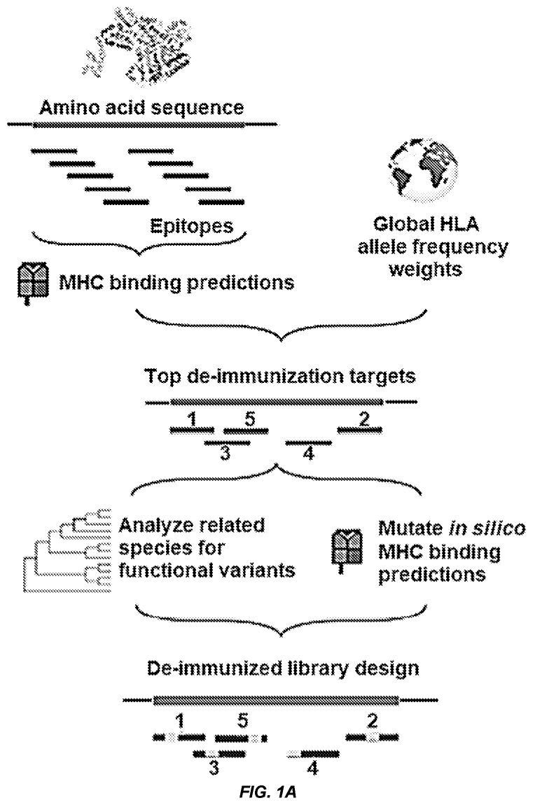

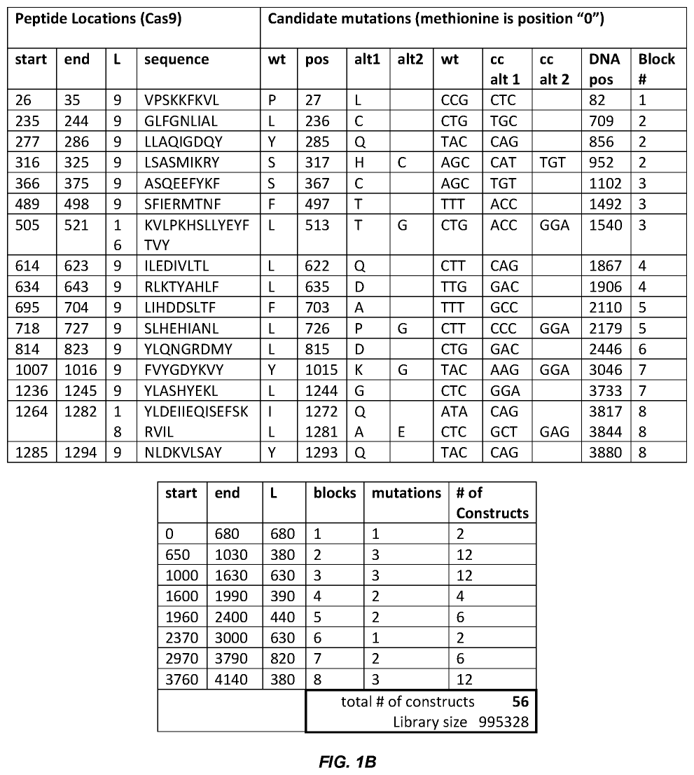

The disclosure provides a 3-part strategy to overcome library size constraints in both the number of unique members and the length of the mutagenized region. The disclosure provides a protein engineering platform capable of screening millions of combinatorial variants simultaneously with mutations spread across the full length of arbitrarily large proteins, with computation-guided mutation design to maximize the probability of exploring functional mutation space (FIG. 1A). In addition to these advantages, the platform can be applied iteratively to tackle particularly challenging engineering tasks by exploring huge swaths of combinatorial mutation space unapproachable using previous techniques. Furthermore, while this methodology is particularly suited to the unique challenges of protein de-immunization, which is demonstrated here as a proof-of-concept use case, it is also applicable to any potential protein engineering goal, so long as there exists an appropriate screening procedure to select for the desired protein functionality. The specific innovations which enable this technology are threefold: library design, library construction, and sequencing readout linking genotype to phenotype.

First, target regions were selected within the protein of interest using software which predicts HLA-binding and peptide immunogenicity. It can be difficult to functionalize these predictions, however, because HLA loci are highly polymorphic, and each HLA allele will have its own particular ligand binding profile. To generalize immunogenicity predictions and select appropriate targets, an approximation of global HLA allele frequencies was created using data from the Allele Frequency Net Database. These frequencies were used to scale immunogenicity predictions such that the top hits are the peptides likely to be the most immunogenic epitopes for the largest number of people globally.

Next, in order to narrow down the vast mutational space associated with fully degenerate combinatorial libraries, an approach guided by evolution and natural variation was utilized. As deimmunizing protein engineering seeks to alter the amino acid sequence of a protein without disrupting functionality, it would be extremely useful to narrow down mutations to those less likely to result in non-functional variants. Mutants were identified by leveraging the large amounts of sequencing data available to identify low-frequency SNPs that have been observed in natural environments. Such variants are likely to have limited effect on protein function, as highly deleterious alleles would likely be immediately selected out of the natural population and not appear in sequencing data. Using these likely neutral amino acid substitutions in combinatorial libraries should substantially increase the likelihood of functional hits with enough epitope variation to evade immune induction. Once the targets are identified and the mutations defined, the library is assembled piecewise using standard synthesis and assembly methods and apply the screen.

In order to read out the library containing mutations dispersed along a long sequence, a long read nanopore sequencing system was utilized. This circumvents the limit of short target regions and obviates the need for barcodes altogether by single-molecule sequencing of the entire target gene. The adoption of nanopore sequencing has been limited by its high error rate compared to established short read techniques; however, careful library design can yield multiple nucleotide changes for each single target amino acid change, effectively increasing the sensitivity of nanopore based readouts exponentially with increasing numbers of nucleotide changes per library member. The large majority of amino acid substitutions are amenable to a library design paradigm in which each substitution is encoded by two, rather than one, nucleotide change, due to the degeneracy of the genetic code and the highly permissive third “wobble” position of codons. For example, if the wild-type amino acid leucine is encoded by the codon CTG, typically a substitution to the amino acid proline would be encoded by the single nucleotide change T to C at the second position, resulting in a CCG codon. However, it is also possible to use any of the other three codons encoding proline, CCT, CCC, and CCA, each of which is two nucleotide changes away from the wild-type sequence. These changes are much easier to reliably detect with error-prone long read nanopore sequencing.

Disclosed herein are methods for identifying or modifying a protein sequence to reduce immunogenicity, and optionally be immunosilent. The method comprises, consists of, or consists essentially of identifying targeted regions of a protein associated with HLA binding. The targeted regions can be ranked by HLA allele frequency using data from the Allele Frequency Net Database ([www.]allelefrequencies.net; brackets provided to eliminate hyperlinks). The frequencies are used for immunogenicity predictions, such that the top hits are the peptides likely to be the most immunogenic epitopes for the largest number of people globally. Next, mutational variants are narrowed by identifying mutations that have the least disruption to protein function. These mutations are identified by sequence comparison analysis using various databases available to the public. Using the databases, low frequency SNPs that have been observed in natural environments are then identified. These SNP variants are likely to have limited effect on protein function; as highly deleterious alleles would likely be immediately selected out of the natural population and not appear in sequencing data. The amino acid variant is identified and these substitutions are used and screened for functional activity and for the ability to generate an immune response.

It was postulated that the disclosure's library design principles informed by natural variation, and long read nanopore sequencing readouts, would allow for the reliable mapping of genotype to phenotype in large scale combinatorial screens of mutations scattered across the full length of a gene. The information obtained from these screens will allow for evaluation of the effects of epistasis, and allow for the tackling of design problems not amenable to current screening technologies, such as full-length epitope deletion and de-immunization.

Disclosed herein are methods for modifying a sequence of a protein or virus to reduce immunogenicity, and optionally be immunosilent. In a particular embodiment, the disclosure provides a method for engineering a protein or virus to be less immunoreactive, comprising one or more of the following steps: identifying target regions of the DNA sequence of protein or virus that are predicted to have human leukocyte antigen (HLA)-binding and/or peptide immunogenicity; identifying single nucleotide polymorphisms (SNPs) or mutations in the targeted region and other regions that are not deleterious to the functioning of the protein; screening a library assembled using standard synthesis and assembly methods by applying the above identifying criteria to find functional variants of the protein or virus; sequencing the functional variants of the protein or virus; and/or mapping genotype to phenotype from the sequences of the functional variants to identify variant candidates that are likely functionally active and have mutations that result in the protein or virus exhibiting less immunogenicity.

The disclosure contemplates use of the methods of the disclosure for reducing the immunogenicity of a protein can be applied to a variety of proteins that present a risk of eliciting an immune response. Non-limiting exemplary proteins of interest include cytidine deaminases, which can be used for gene editing via catalysis of DNA base change from C to T (e.g. APOBEC—Conserved across many species e.g. Rat APOBEC3, Rat APOBEC1, Resus Macaque APOBEC3G, human APOBEC1 (A1), AID, APOBEC2 (A2), APOBEC3A (A3A), APOBEC3B (A3B), APOBEC3C (A3C), APOBEC3DE (A3DE), APOBEC3F (A3F), APOBEC3G (A3G), APOBEC3H (A3H) and APOBEC4 (A4)); adenosine deaminases, which can be used for gene editing via catalysis of DNA base change from A to G (e.g. ADA (DNA editor)—Widely conserved across virtually all species and ADAR (RNA editor)—Conserved across most metazoan species); Zing Finger nucleases (ZFNs), which can be used for genome engineering in a similar manner to CRISPR/Cas9 and are engineered site-specific nucleases consisting of: 3-6 repeated zinc finger domains, which is a widely conserved DNA-binding motif and a nuclease domain; transcriptional activator-like effector nucleases (TALENs), which be used for genome engineering in a similar manner to CRISPR/Cas9 and are similar to ZFNs in that they are engineered site-specific nucleases consisting of: a TAL effector DNA binding domain (generally derived from a species of Xanthomonas proteobacteria) and a nuclease domain. The domains of the site-specific enzymes mentioned above (ZFNs and TALENs) are well characterized and subject of extensive engineering to generate the desired specificity. Thus, many variants exist of such proteins. Additional proteins for which HLA-binding affinity analysis is relevant include Cas9 proteins and AAV capsids, both of which are used in CRISPR based gene editing.

In a particular embodiment, the methods disclosed herein provide for reducing the immunogenicity of a CRISPR associated protein. Examples of CRISPR associated proteins include, but are not limited to, Cas9, Cas12, Cas13, Cas14. In yet another embodiment, the CRISPR associated protein is a Cas9. In a further embodiment, the Cas9 is Streptococcus pyogenes Cas9 (SpCas9). In some embodiments, the Cas9 proteins the orthologs are selected from S. pyogenes Cas9 (spCas9), S. aureus Cas9 (saCas9), B. longum Cas9, A. muiciniphilia Cas9, or O. laneus Cas9. In order to optimize and broaden the application of CRIPSR based therapeutics the disclosure provides methods to “humanize” the CRISPR associated protein by swapping high immunogenic domains or peptides with less immunogenic counterparts. This is particularly useful to enable the application of CRIPSR based therapeutics for repeat treatments. The disclosure teaches methods and methodology to screen mutations in selected targeted regions of proteins, such as CRISPR associated proteins, in order to reduce immunogenicity.

Thus, embodiments of the disclosure relate to a modified CRISPR associated protein that has lower immunogenicity to promote immune evasion. The modified proteins can replace existing wildtype proteins for any application requiring in vivo delivery, which would potentially have no loss of efficacy after repetitive use.

In some aspects, provided herein are isolated polynucleotides encoding a modified Cas9 protein, wherein the modified Cas9 comprises, consists of, or consists essentially of one or more, two or more, three or more, four or more, five or more, six or more, seven or more, eight or more, nine or more, ten or more, fifteen or more, or twenty or more of the amino acid modifications in targeted regions to lower the immunogenicity of the protein. In one embodiment, the disclosure provides an engineered immune-silenced Cas9 comprising a sequence of SEQ ID NO:2 and having one or more mutations selected from the group consisting of: P28L, L237C, Y286Q, S318(H/C), S368C, F498T, L514(T/G), L616G, L623Q, L636D, F704A, L727(P/G), L816D, Y1016(K/G), L1245G, I1273Q, L1282(A/E), and/or Y1294Q. In some aspects, provided herein are vectors comprising an isolated polynucleotide encoding an engineered Cas9 comprising one or more mutations selected from the group consisting of: P28L, L237C, Y286Q, S318(H/C), S368C, F498T, L514(T/G), L616G, L623Q, L636D, F704A, L727(P/G), L816D, Y1016(K/G), L1245G, I1273Q, L1282(A/E), and/or Y1294Q. In some embodiments, the vector is an AAV vector, optionally wherein the AAV vector is AAV5.

The disclosure provides an isolated polypeptide comprising (i) SEQ ID NO:2 having a mutation(s) selected from P28L, L237C, Y286Q, S318(H/C), S368C, F498T, L514(T/G), L616G, L623Q, L636D, F704A, L727(P/G), L816D, Y1016(K/G), L1245G, I1273Q, L1282(A/E), Y1294Q, and any combination thereof, wherein the polypeptide has Cas9 activity and wherein the polypeptide has reduced immunogenicity compared to SEQ ID NO:2 lacking any one or more of the mutations; (ii) a sequence that is 95%-99% identical to (i); and (iii) related homolog/orthologs having mutations corresponding to the mutations in SEQ ID NO:2 and having Cas9 activity. The disclosure also provided isolated polynucleotides encoding the polypeptide of (i)-(iii) above. The polynucleotides can be RNA or DNA. The polynucleotides can be cloned into a vector for expression and/or delivery to a subject.

In yet a further embodiment, the targeted regions to lower the immunogenicity of the protein are identified using a model that predicts human leukocyte antigen (HLA)-binding and peptide immunogenicity. Models for determining HLA-binding affinity and peptide immunogenicity are likewise known in the art and may include computational methods available through software or publicly accessible databases or “wet lab” assays. Examples of computational methods of predicting HLA-binding affinity include, but are not limited to, the MHC prediction models available through the IEDB Analysis Resource ([http://]tools.immuneepitope.org/mhci/ (MHC I) and [http://]tools.immuneepitope.org/mhcii/ (MHC II)) or NetMHC ([http://www.]cbs.dtu.dk/services/NetMHC/). Other examples of prediction models include, but are not limited to, NetMHC, MHCAttnNet, MHCSeqNET, ACME, NetMHCpan EL 4.1, NetMHCstabpan, SMM, SMMPMBEC, PickPocket, Comblib_Sidney2008, NetMHCcons, MHCflurry 2.0, and IConMHC. Alternatively or in addition, HLA-binding can be determined or computational predictions thereof can be validated using assays, such as, but not limited to, immunoassays, such as ELISA, microarray, tetramer assay, and peptide-induced MHC stabilization assay. Using such assays and computational methods can further be adapted to account for immune response of a specific subject or patient being treated. Thus, modifications in the proteins can be optimized to reduce the immunogenicity of the protein when administered to a particular subject or patient. Similarly, the comparisons can be host-restricted, such that the protein is optimized to reduce the immunogenicity of the protein when administered to a particular host, e.g., a mouse or a human. Examples of such, include “humanizing” the protein by swapping high immunogenic domains or peptides with less immunogenic counterparts.

In order to narrow down the vast mutational space associated with fully degenerate combinatorial libraries, an approach guided by evolution and natural variation is utilized. As deimmunizing protein engineering seeks to alter the amino acid sequence of a protein without disrupting functionality, it would be extremely useful to narrow down mutations to those less likely to result in non-functional variants. These mutants are identified by leveraging the large amounts of sequencing data available to identify low-frequency SNPs and mutations that have been observed in natural environments. Such variants are likely to have limited effect on protein function, as highly deleterious alleles would likely be immediately selected out of the natural population and not appear in sequencing data. In a particular embodiment, SNPs or mutations are identified by using phylogenetic methods to scan natural variation among naturally occurring proteins, mutations generated in the course of research and engineering efforts, and protein orthologs from closely related species. In yet another embodiment, the SNPS or mutations are identified, or further identified, by using immunological prediction of candidate mutations to ensure significant loss of immunogenicity within the targeted region in order to preserve function while reducing immunogenicity.

The disclosure contemplates use of the methods of the disclosure for reducing the immunogenicity of viruses. The methods can be applied to a variety of types of viruses that present a risk of eliciting an immune response, particularly those used in gene therapy or gene delivery. Examples of such viruses, include but are not limited to, retroviruses, adenoviruses, adeno-associated viruses (AAVs), alphaviruses, lentiviruses, pox viruses, and herpes viruses. In a further embodiment, the virus is an AAV. In yet a further embodiment, the AAV is selected from AAV1, AAV2, AAV5, AAV6, AAV7, and AAV8. In another embodiment, the AAV is AAV5.

The disclosure provides methods encompassing a step of identifying target regions of proteins and the corresponding polynucleotide coding sequence of a virus that are predicted to have human leukocyte antigen (HLA)-binding and/or peptide immunogenicity target regions. In a further embodiment, the targeted regions are identified by aligning conserved sequence regions across AAV serotypes. In a further embodiment, the SNPs are identified by aligning the sequences of 50, 60, 70, 80, 90, 100, 110, 120, 130, 140, 150, 160, 170, 180, 190, 200 or more AAV variants that have been sequenced from natural or engineered sources. In yet a further embodiment, the SNPs are located in the target regions.

In another embodiment, in order to identify and characterize the function of variant protein or viral candidates that are likely functionally active and have mutations that result in the protein or virus exhibiting less immunogenicity, long-read sequencing technologies capable of sequencing the entire sequence of each protein or viral variant in one sequencing reaction are used. In yet another embodiment, the long-read sequencing technologies is capable of generating 10-15 Gb of sequencing reads per run. Examples of such sequencing technologies include, but are not limited to, Oxford Nanopore's MinIon sequencer.

In a further embodiment, a method disclosed herein encompasses the step of evaluating the immunoreactivity of variant candidates in one or more immunoassays. In yet a further embodiment, the one or more immunoassays comprise detecting the presence of antibodies to the variant candidates (AVA antibodies), when the variant candidates are administered in vivo to an animal. In another embodiment, the one or more immunoassays comprise an enzyme-linked immunosorbent assays (ELISAs), electrochemiluminescence (ECL) assays and/or antigen-binding tests, wherein the one or more immunoassays utilize AVA antibodies.

The disclosure also provide composition used in various therapies wherein the composition comprises a potentially immunogenic molecule such as a protein or polypeptide. The methods of the disclosure can be used to identify domains that are immunogenic and identify mutations that reduce immunogenicity. For example, Cas9 is a protein used in gene editing in vivo, but has been shown to have immunogenic potential. In a particular embodiment, the disclosure provides for a polynucleotide having at least 90%, 95%, 97%, 98%, or 99% sequence identity to the sequence presented in SEQ ID NO:1, wherein the polynucleotide encodes a polypeptide having at least 90% identity to SEQ ID NO:2 having reduced immunogenicity and wherein the protein has Cas9 like activity. In another embodiment, the disclosure also provides for a protein having a polypeptide sequence that has at least 90%, 95%, 97%, 98%, or 99% sequence identity to the sequence presented in SEQ ID NO:2, wherein the protein has Cas9 like activity. In a further embodiment, the protein has less immunogenicity than Cas9.

In a certain embodiment, the disclosure further provides for a CRISPR-Cas9 system comprising a protein disclose herein that has Cas9 like activity. In another embodiment, the CRISPR-Cas9 system further comprises RNA that comprise a shot sequence that binds to a specific target sequence of DNA in a genome. It is appreciated by those skilled in the art that RNAs can be generated for target specificity to target a specific gene, optionally a gene associated with a disease, disorder, or condition. Thus, in combination with Cas9, the guide RNAs facilitate the target specificity of the CRISPR/Cas9 system. Further aspects such as promoter choice, may provide additional mechanisms of achieving target specificity—e.g., selecting a promoter for the guide RNA encoding polynucleotide that facilitates expression in a particular organ or tissue. Accordingly, the selection of suitable RNAs for the particular disease, disorder, or condition is contemplated herein.

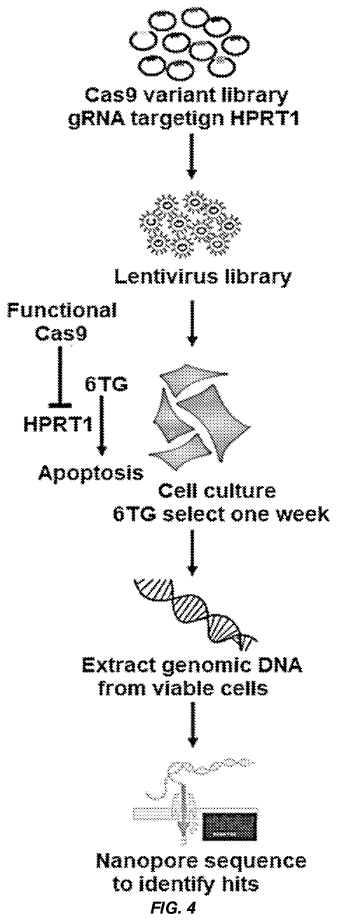

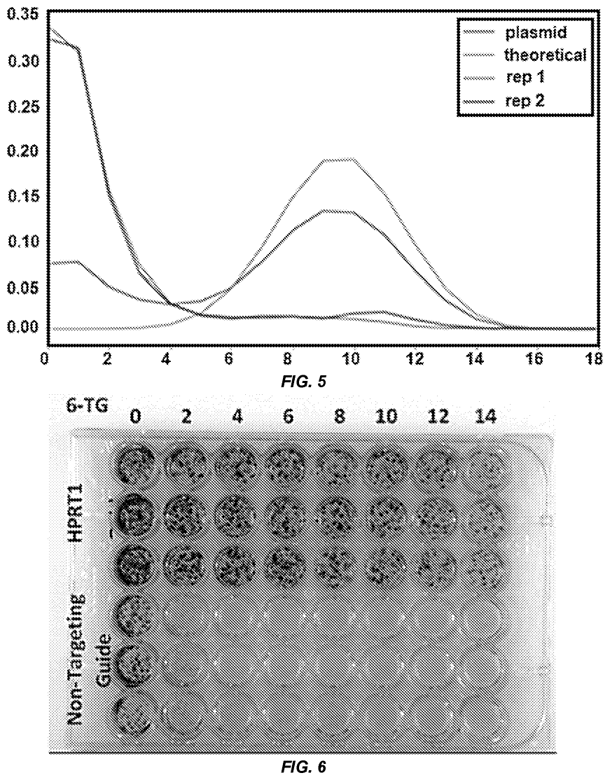

In a particular embodiment, the disclosure provides for a virus encoded by a polynucleotide sequence having at least 90%, 95%, 97%, 98%, or 99% sequence identity to the sequence presented in FIG. 5, wherein the virus has AAV5 like activity. In a certain embodiment, the disclosure also provides for a virus capsid having at least 90%, 95%, 97%, 98%, or 99% sequence identity to the sequence presented in SEQ ID NO:50, wherein the virus has AAV5 like activity. In another embodiment, the virus has less immunogenicity than AAV5.

In a certain embodiment, the disclosure also provides for an AAV system comprising a virus disclosed herein that has AAV5 like activity. In a further embodiment, the AAV system is used for gene therapy. Administration of the AAV variant or compositions thereof can be affected in one dose, continuously or intermittently throughout the course of treatment. Administration may be through any suitable mode of administration, including but not limited to: intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmuccosal, and inhalation.

Methods of determining the most effective route and dosage of administration are known to those of skill in the art and will vary with the composition used for therapy, the purpose of the therapy and the subject being treated. Single or multiple administrations can be carried out with the dose level and pattern being selected by the treating physician. It is noted that dosage may be impacted by the route of administration. Suitable dosage formulations and methods of administering the agents are known in the art. Non-limiting examples of such suitable dosages may be as low as 1 E+9 vector genomes to as much as 1 E+17 vector genomes per administration.

In a further embodiment, a modified virus and compositions of the disclosure having reduced immunogenicity can be administered in combination with other treatments, e.g. those approved treatments suitable for the particular disease, disorder, or condition.

Doses suitable for uses herein may be delivered via any suitable route, e.g. intravenous, transdermal, intranasal, oral, mucosal, or other delivery methods, and/or via single or multiple doses. It is appreciated that actual dosage can vary depending on the recombinant expression system used (e.g. AAV or lentivirus), the target cell, organ, or tissue, the subject, as well as the degree of effect sought. Size and weight of the tissue, organ, and/or patient can also affect dosing. Doses may further include additional agents, including but not limited to a carrier. Non-limiting examples of suitable carriers are known in the art: for example, water, saline, ethanol, glycerol, lactose, sucrose, dextran, agar, pectin, plant-derived oils, phosphate-buffered saline, and/or diluents. Additional materials, include those disclosed in paragraph [00533] of WO 2017/070605 may be used with the compositions disclosed herein. Paragraphs [00534] through [00537] of WO 2017/070605 also provide non-limiting examples of dosing conventions for CRISPR-Cas systems which can be used herein. In general, dosing considerations are well understood by those in the art.

The disclosure also provides for compositions or kits comprising any one or more of the variant proteins and/or variant viruses described herein. In one embodiment, the carrier is a pharmaceutically acceptable carrier. These compositions can be used therapeutically as described herein and can be used in combination with other known therapies and/or according to the method aspects described herein.

Briefly, pharmaceutical compositions of the present invention may comprise a variant Cas9 described herein or a polynucleotide encoding said Cas9, optionally comprising a variant AAV described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure may be formulated for oral, intravenous, topical, enteral, and/or parenteral administration. In certain embodiments, the compositions of the present disclosure are formulated for intravenous administration.

The following examples are intended to illustrate but not limit the disclosure. While they are typical of those that might be used, other procedures known to those skilled in the art may alternatively be used.

### Examples

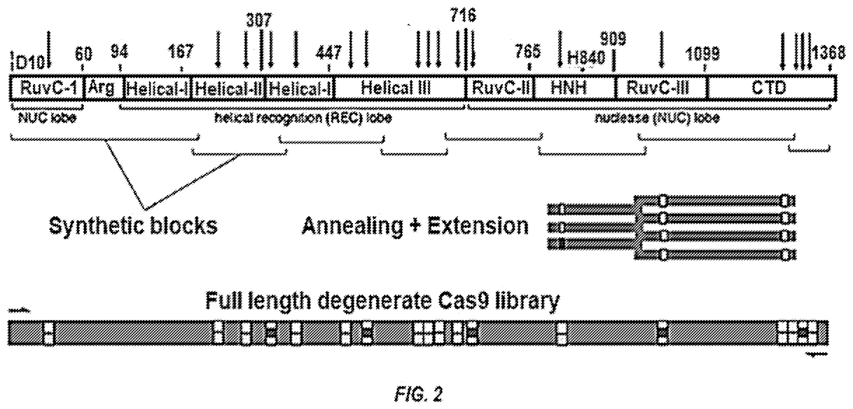

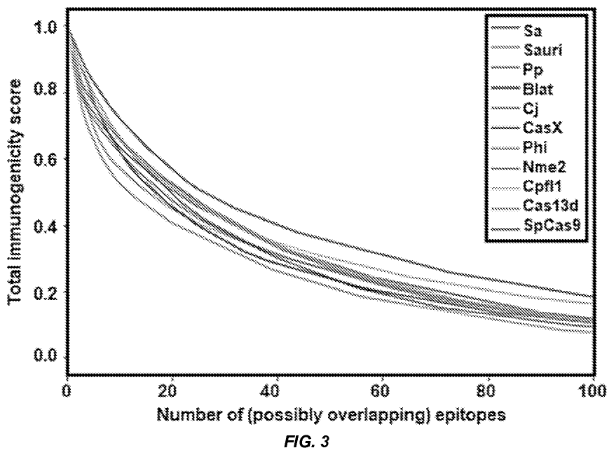

Applying the procedure described herein, a library of Cas9 variants were designed based on the SpCas9 backbone containing 23 different mutations across 17 immunogenic epitopes (FIG. 2A) (e.g., P28L, L237C, Y286Q, S318(H/C), S368C, F498T, L514(T/G), L616G, L623Q, L636D, F704A, L727(P/G), L816D, Y1016(K/G), L1245G, I1273Q, L1282(A/E), and/or Y1294Q; relative to SEQ ID NO:2). Combining these in all possible combinations yields a library of 1,492,992 unique elements. With this design in hand, a library was constructed in a stepwise process. First, the full-length gene was broken up into short blocks of no more than 1000 bp, which overlap by 30 bp on each end. Each block is designed such that it contains no more than 3 or 4 target epitopes to mutagenize. With few epitopes per block and few variant mutations per epitope, it becomes feasible to chemically synthesize each combination of mutations for each block. For example, block 2 of the SpCas9 library contains 3 target epitopes (see, e.g., FIG. 2B). One variant mutation was designed to test at two of these sites, and two variant mutations at the final site. These variants plus the wild-type result in a total of 2*2*3=12 combinations. Each of these combinations is then synthesized and mixed at equal ratios to make a degenerate block mix. This is repeated for each of the blocks necessary to complete the full-length protein sequence.

Subsequently, each of these block mixes were assembled into a fully combinatorial library of Cas9 sequences using a two-step PCR assembly method. The first annealing-extension step allows each of the blocks to anneal to and prime their attachment to neighboring blocks using their 30 bp overlapping ends. DNA polymerase then extends these attached fragments into full length Cas9 genes with the order of block assembly being specified by the unique 30 bp overhangs at each block junction. In the second step, primers binding to only the far 3′ and 5′ ends of the Cas9 sequence are used to amplify the full length Cas9 library in a standard PCR reaction. The library is then purified and inserted into an appropriate cloning/expression vector via Gibson assembly.

Each of these components of library design and construction, the selection of target sites, the identification of high-likelihood functional mutations, the multiple nucleotide mutation scheme, the subdivision of the protein into blocks, and the assembly of those blocks into a fully degenerate combinatorial library, come together to enable the functionality of this protein engineering platform. To demonstrate the utility of this methodology, a combinatorial library screen was generated to heavily de-immunize the Streptococcus pyogenes Cas9 nuclease (SpCas9; SEQ ID NO:1 and 2, polynucleotide and polypeptide, respectively). This classical Cas9 is currently under clinical development for use in gene therapy, but being of microbial origin, indeed, from a species which opportunistically causes human disease, this protein may be expected to generate an immune response which can inhibit therapeutic efficacy.

After construction of the Cas9 variant library, nanopore sequencing was applied using the Oxford Nanopore (ONT) MinION platform. To generate the sequencing library, PCR amplification was performed on the full-length Cas9 gene from the plasmid library preparation and the nanopore sequencing adapters were ligated as per standard ONT protocol. Using a single MinION flow cell, a 1× sequencing depth of the library was performed, which was sufficient to serve as a QC check and to ensure library diversity. Although the low sequencing depth and noisy nature of nanopore reads only allowed reliable identification of 304,060 unique library elements. The read count distribution suggests that this is an under-sampling of the library diversity, and the mutation density distribution, i.e., the number of detected elements with each possible number of mutations closely matches theoretical expectation, except for a slight oversampling of sequences with small numbers of mutations. In spite of this, the majority of the pre-screen library consists of Cas9 sequences with significant numbers of mutations, with most falling into a broad peak between 6 and 14 mutations per sequence (FIG. 5).

To identify functional variants still capable of editing DNA, a positive screen targeting the hypoxanthine phosphor-ribosyltransferase 1 (HPRT1) gene was designed and tested. In the context of the screen, HPRT1 converts 6-thioguanine (6TG), an analogue of the DNA base guanine, into 6-thioguanine nucleotides that are cytotoxic to cells via incorporation into the DNA during S-phase. Thus, only cells containing functional Cas9 variants capable of disrupting the HPRT1 gene can survive in 6TG (FIG. 4).

To first identify the optimal 6TG concentration, HeLa cells were transduced with lentivirus particles containing wild-type Cas9 and either a HPRT1-targeting guide RNA (gRNA) or a non-targeting guide. After selection with puromycin, cells were treated with 6TG concentrations ranging from 0-14 μg/mL for one week. Cells were stained with crystal violet at the end of the experiment and imaged. 6 μg/mL was selected as all cells containing non-targeting guide had died while cells containing the HPRT1 guide remained viable (FIG. 6).

To perform the screen, HeLa cells were transduced with lentiviral particles containing variant library or wild-type Cas9 along with the HPRT1-targeting gRNA at 0.3 MOI and at greater than 75-fold coverage of the library elements. Cells were selected using puromycin after two days and 6TG was added once cells reached 75% confluency. After two weeks, genomic DNA was extracted from remaining cells and full-length Cas9 sequences were PCR amplified. Nanopore-compatible sequencing libraries were prepared per manufacturer's instructions and sequenced on the MinION platform. This screening procedure was performed in two replicates.

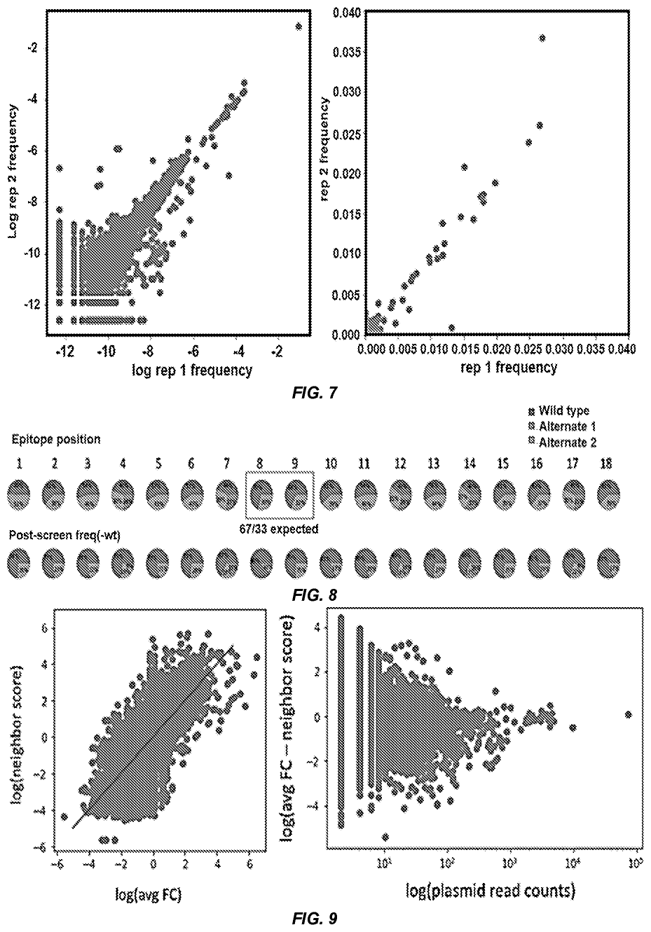

After screening, the library was significantly shifted in the mutation density distribution, suggesting that the majority of the library with large (>4) numbers of mutations resulted in non-functional proteins which were unable to survive the screen. Meanwhile, wild-type, single, and double mutants were generally enriched as these proteins proved more likely to retain functionality and pass through the screen (FIG. 5). Additionally, the two independent replicates of the screen showed strong correlation (R²=0.925) providing further evidence of the robustness of the screen (FIG. 7).

In addition, the overall frequency of mutations in the pre- and post-screen libraries was analyzed to see if a pattern of mutation effects could be inferred. Although the wild-type allele was enriched at every site in the post-screen sequences, nearly every site retained a significant fraction of mutated alleles, suggesting that the mutations, at least individually, are fairly well-tolerated and do not disrupt Cas9 functionality (FIG. 8).

In order to select hits from the screen for downstream validation and analysis, a method for differentiating high-support hits likely to be real from noise-driven false positive hits was devised. To do this it was hypothesized that the fitness landscape of the screen mutants is likely to be smooth, i.e. variants that contain similar mutations are more likely to have similar fitnesses in terms of editing efficiency compared to randomly selected pairs. This was confirmed by computing a predicted screen score for each variant based on a weighted regression of its nearest neighbors in the screen. This metric correlates well with the actual screen scores and approaches the screen scores even more closely as read coverage increases. This provides good evidence that the fitness landscape is indeed somewhat smooth (FIG. 9).

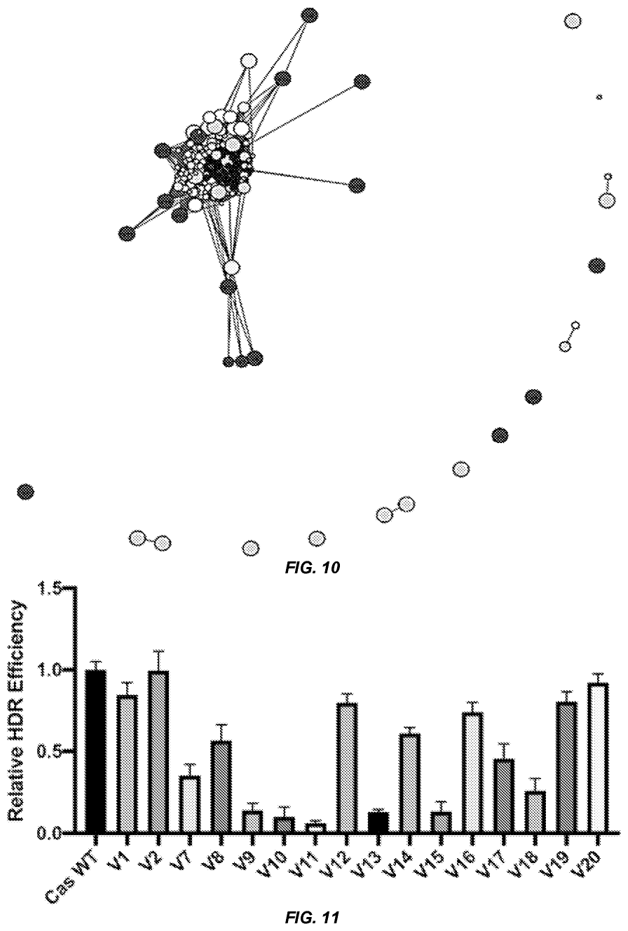

Next, a network analysis was performed to try to differentiate noise-driven hits from bona fide hits by looking at the degree of connectivity with other hits. The rationale here is that because the fitness landscape is smooth, real hits should reside in broad fitness peaks including many neighbors that also show high screen scores, whereas hits that are less supported by near neighbors are more likely to be spurious as they represent non-smooth fitness peaks (FIG. 10).

To validate and characterize hits from the screen, two independent methods were applied to quantify editing of the de-immunized Cas9 variants. First, a gene-rescue experiment was performed using low frequency homology directed repair (HDR) to repair a genetically encoded broken green fluorescent protein (GFP) gene. Upon successful editing and co-transfection with a correct donor GFP copy, a fraction of cells will convert to GFP+. Briefly, a HEK293T cell line containing the GFP DNA sequence with the insertion of a stop codon and a genomic fragment of the AAVS1 locus was developed. Due to the stop codon, this line is naturally nonfluorescent. However, GFP expression can be restored via homologous recombination, where the DNA is edited via a guide that targets the AAVS1 locus fragment and repaired with a GFP donor sequence. GFP+ cells can then be quantified by flow-activated cell sorting (FACS), providing information on editing efficiency. Second, editing was quantified by genomic DNA extraction and Illumina next generation sequencing (NGS) using the CRISPResso package.

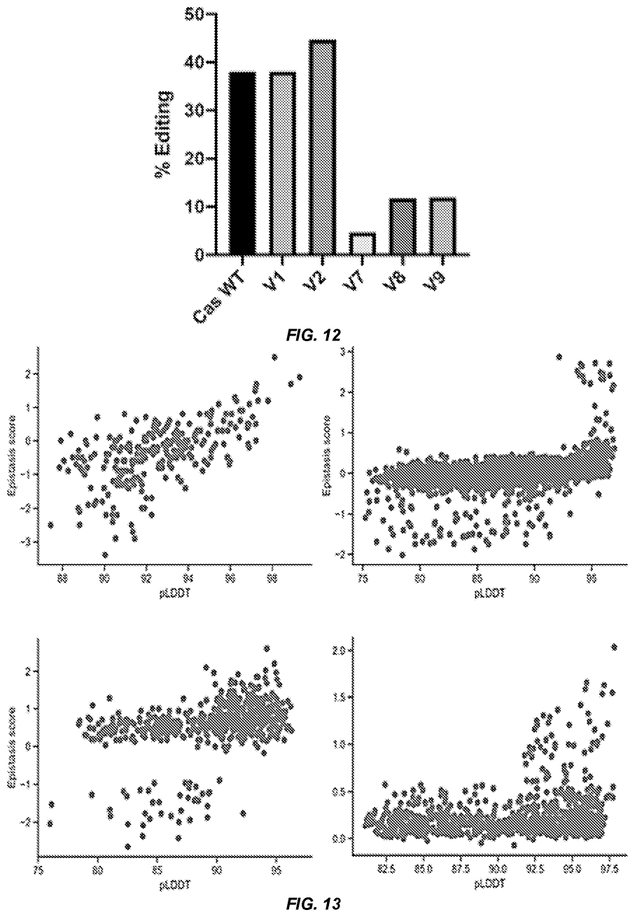

To specifically validate this network, 20 variants (V1-20) were constructed as detailed in Table A. A large majority of the variants were capable of editing, providing further confidence in the network we constructed, and in particular highlighted variants V8 and V12 with high editing capability and 8 and 7 mutations respectively (FIGS. 11 and 12). Leveraging the unique combinatorial library design and screening strategy, Cas9 variants were generated with multiple top immunogenic epitopes simultaneously de-immunized while retaining editing functionality.

TABLE A
De-Immunized Cas Variant Hits (mutational numbering
excludes amino acid 1 (Met) with respect to SEQ ID NO: 2.

| ID | 1 | 2 | 3 | 4 | 5 | 6 | 7 | 8 | 9 |
|---|---|---|---|---|---|---|---|---|---|
| V1 | — | — | — | — | — | — | — | L615G | — |
| V2 | — | — | — | — | — | — | — | — | L622Q |
| V3 | — | — | — | S317H | S367C | — | — | — | — |
| V4 | P27L | — | — | S317H | S367C | F497T | — | L615G | — |
| V5 | — | — | Y285Q | S317C | — | F497T | — | — | — |
| V6 | — | — | — | S317C | — | — | — | — | L622Q |
| V7 | P27L | — | Y285Q | — | S367C | — | L513T | — | L622Q |
| V8 | — | — | Y285Q | — | S367C | — | L513T | — | L622Q |
| V9 | — | L236C | — | — | S367C | F497T | — | L615G | — |
| V10 | — | — | Y285Q | S317H | S367C | — | L513T | — | L622Q |
| V11 | — | — | — | — | — | — | — | — | L622Q |
| V12 | — | — | Y285Q | — | — | — | L513T | — | L622Q |
| V13 | — | L236C | — | S317H | — | — | — | — | — |
| V14 | — | L236C | — | — | S367C | F497T | — | L615G | — |
| V15 | — | — | — | S317C | — | — | L513T | — | — |
| V16 | — | — | — | — | — | F497T | — | — | L622Q |
| V17 | — | — | — | — | S367C | — | — | — | L622Q |
| V18 | — | — | — | — | — | — | — | — | L622Q |
| V19 | — | — | — | S317H | — | F497T | L513G | — | L622Q |
| V20 | — | — | — | — | — | — | L513T | — | L622Q |
| ID | 10 | 11 | 12 | 13 | 14 | 15 | 16 | 17 | 18 |
| V1 | — | — | — | — | — | — | — | — | — |
| V2 | — | — | — | — | — | — | — | — | — |
| V3 | — | F703A | L726G | L815D | Y1015G | L1244G | I1272Q | L1281A | Y1293Q |
| V4 | — | F203A | — | — | Y1015K | — | I1272Q | L1281A | Y1293Q |
| V5 | L635D | F703A | L726P | — | Y1015G | L1244G | — | L1281A | — |
| V6 | L635D | F703A | L726P | L815D | Y1015K | — | — | L1281A | Y1293Q |
| V7 | — | — | L726G | L815D | — | L1244G | — | L1281A | — |
| V8 | — | — | L726G | L815D | — | L1244G | — | L1281A | — |
| V9 | — | — | — | — | Y1015G | — | I1272Q | — | Y1293Q |
| V10 | — | — | L726G | L815D | — | L1244G | — | L1281A | — |
| V11 | L635D | F703A | — | L815D | — | L1244G | I1272Q | L1281E | — |
| V12 | — | — | L726G | L815D | — | L1244G | — | L1281A | — |
| V13 | — | — | — | L815D | — | — | I1272Q | L1281E | Y1293Q |
| V14 | — | — | — | — | Y1015G | — | — | — | Y1293Q |
| V15 | L635D | F703A | — | — | Y1015G | — | — | — | Y1293Q |
| V16 | — | — | L726G | — | — | — | — | L1281A | Y1293Q |
| V17 | — | — | L726P | — | Y1015G | — | I1272Q | — | — |
| V18 | L635D | F703A | — | — | Y1015G | L1244G | — | — | — |
| V19 | — | — | — | — | — | — | — | — | — |
| V20 | — | — | — | — | — | — | — | — | — |

In silico Screens. To demonstrate the broad applicability of this combinatorial protein engineering platform, top high-priority immunogenic epitopes were identified, similar to the SpCas9 library, for 10 alternative Cas orthologs (Staphylococcus aureus Cas9 (Accession Number: J7RUA5.1; SEQ ID NO:51 and 52, polynucleotide and polypeptide, respectively), Campylobacter jejuni Cas9 (Accession Number: YP_002344900.1; SEQ ID NO:53), Staphylococcus auricularis Cas9 (Accession Number: WP_107392933.1), CasX (Accession Number: OGP07438.1; SEQ ID NO:54), Cas-Phi (Accession Number: 7LYS_A; SEQ ID NO:55), Cas13d (Accession Number: QMT62609.1), Acidaminococcus Cas12a (Accession Number: U2UMQ6.1; SEQ ID NO:56), Pasteurella pneumotropica Cas9 (Accession Number: WP_018356570.1), Brevibacillus lacterosporus Cas9 (Accession Number: WP_003343632.1), Neisseria meningitidis Cas9 (Accession Number: WP_002260677.1) focusing on small Cas9s amenable to in vivo use via adeno-associated viral vectors (AAVs), and on Cas orthologs which extend the utility of the CRISPR system beyond the Cas9 case, such as the RNA-targeting Cas13d. (Table B; all sequences associated with the accession numbers above are incorporated herein by reference). It will be readily apparent to one of skill in the art that additional immuno-silenced Cas protein constructs can be generated using the information above and in Table B and C in combination with the sequence listings accompanying the application.

TABLE B
Top immunogenic epitopes across Cas9 orthologs (Mutational reference
amino acids exclude position 1 methionine, e.g., P27L excludes methionine
at position 1 of SEQ ID NO: 2 Sp-Cas9; or alternatively stated, methionine
is amino acid ″0″).

| Source | AA position | | |
|---|---|---|---|
| protein | (0-based) | Epitope | Mutation |
| Sp-Cas9 | 26 | VPSKKFKVL | P27L (e.g., P28L of SEQ ID NO: 2) |
| Sp-Cas9 | 235 | GLFGNLIAL | L236C |
| Sp-Cas9 | 277 | LLAQIGDQY | Y285Q |
| Sp-Cas9 | 316 | LSASMIKRY | S317H; S317C; Y324D |
| Sp-Cas9 | 366 | ASQEEFYKF | S367C |
| Sp-Cas9 | 489 | SFIERMTNF | F497T; F490N |
| Sp-Cas9 | 505 | KVLPKHSLLYEYFTVY | L513T; L513G |
| Sp-Cas9 | 614 | ILEDIVLTL | L615G; L622Q |
| Sp-Cas9 | 634 | RLKTYAHLF | L635D; R634E |
| Sp-Cas9 | 695 | LIHDDSLTF | F703A; I696D |
| Sp-Cas9 | 718 | SLHEHIANL | L726P; L726G |
| Sp-Cas9 | 814 | YLQNGRDMY | L815D |
| Sp-Cas9 | 1007 | FVYGDYKVY | Y1015K; Y1015G; Y1015E |
| Sp-Cas9 | 1236 | YLASHYEKL | L1244G |
| Sp-Cas9 | 1264 | YLDEIIEQISEFSKRVIL | I1272Q; L1281A; L1281E |
| Sp-Cas9 | 1285 | NLDKVLSAY | Y1293Q; L1286K |
| Sp-Cas9 | 11 | GTNSVGWAV | T12N |
| Sp-Cas9 | 65 | RTARRRYTR | T66R |
| Sp-Cas9 | 155 | LALAHMIKFRGHFL | M160P |
| Sp-Cas9 | 441 | KILTFRIPYYV | T444E |
| Sp-Cas9 | 805 | LQNEKLYLY | Q806R |
| Sp-Cas9 | 877 | KMKNYWRQL | M878D |
| Sp-Cas9 | 1030 | KATAKYFFYSNIMNFFK | Y1038E |
| Sp-Cas9 | 1086 | LSMPQVNIV | V1094S |
| Sp-Cas9 | 1137 | TVAYSVLVV | V1138K; V1145E |
| Sp-Cas9 | 1211 | RMLASAGEL | L1219N |
| Sa-Cas9 | 4 | YILGLDIGI | I12G; I12N (e.g., I13G of SEQ 52) |
| Sa-Cas9 | 68 | KLLFDYNLL | L76S; L76Q |
| Sa-Cas9 | 104 | FSAALLHLA | A112N |
| Sa-Cas9 | 230 | EMLMGHCTYF | Y238W |
| Sa-Cas9 | 257 | ALNDLNNLV | V265S |
| Sa-Cas9 | 349 | QIAKILTIY | Y357N |
| Sa-Cas9 | 388 | YTGTHNLSLKAINLI | L396T; L396G |
| Sa-Cas9 | 408 | HTNDNQIAIFNRL | T409G; I416Q |
| Sa-Cas9 | 455 | QSIKVINAI | S456G |
| Sa-Cas9 | 556 | HIIPRSVSFDNSF | F564A |
| Sa-Cas9 | 650 | YATRGLMNL | L658T |
| Sa-Cas9 | 660 | RSYFRVNNL | S661D |
| Sa-Cas9 | 674 | SINGGFTSF | F682K; F682G |
| Sa-Cas9 | 706 | IIANADFIFK | I707C |
| Sa-Cas9 | 735 | KQAESMPEI | I743Q |
| Sa-Cas9 | 800 | IVNNLNGLY | Y808T |
| Sa-Cas9 | 916 | YLDNGVYKF | F924E; F924G |
| Sa-Cas9 | 957 | NQAEFIASF | Q958K |
| Sa-Cas9 | 972 | KINGELYRV | V980Q |
| Sa-Cas9 | 992 | EVNMIDITY | Y1000E |
| Sa-Cas9 | 1014 | RIIKTIASKTQSI | K1022C |
| As-Cas12a | 1 | TQFEGFTNL | — |
| As-Cas12a | 159 | RSFDKFTTYFSGF | — |
| As-Cas12a | 209 | RLITAVPSL | — |
| As-Cas12a | 234 | TSIEEVFSFPFYNQL | — |
| As-Cas12a | 356 | NSIDLTHIF | — |
| As-Cas12a | 472 | SLLGLYHLL | — |
| As-Cas12a | 556 | FVKNGLYYL | — |
| As-Cas12a | 678 | FTRDFLSKY | — |
| As-Cas12a | 716 | LLYHISFQR | — |
| As-Cas12a | 760 | HTLYWTGLF | — |
| As-Cas12a | 793 | RMKRMAHRL | — |
| As-Cas12a | 929 | RSLNTIQQF | — |
| As-Cas12a | 970 | YLSQVIHEI | — |
| As-Cas12a | 981 | LMIHYQAVVV | — |
| As-Cas12a | 1017 | MLIDKLNCL | — |
| As-Cas12a | 1034 | KVGGVLNPY | — |
| As-Cas12a | 1055 | GTQSGFLFYVPAPY | — |
| As-Cas12a | 1131 | FMPAWDIVF | — |
| As-Cas12a | 1209 | HAIDTMVALIRSVL | — |
| As-Cas12a | 1292 | ISNQDWLAY | — |
| Blat-Cas9 | 0 | MAYTMGIDVGI | — |
| Blat-Cas9 | 191 | LLVEIHTLF | — |
| Blat-Cas9 | 236 | KMIGTCTFL | — |
| Blat-Cas9 | 252 | KASWHFQYF | — |
| Blat-Cas9 | 261 | MLLQTINHI | — |
| Blat-Cas9 | 343 | KLNKIFNEV | — |
| Blat-Cas9 | 366 | TVAYALTFFK | — |
| Blat-Cas9 | 404 | YTNELIGKV | — |
| Blat-Cas9 | 426 | KALRKIIPFL | — |
| Blat-Cas9 | 439 | MTYDKACQA | — |
| Blat-Cas9 | 583 | HIIPYSRSM | — |
| Blat-Cas9 | 669 | YITKYLSHFISTNLEF | — |
| Blat-Cas9 | 722 | AMDAIVIAV | — |
| Blat-Cas9 | 735 | FIQQVTNYYK | — |
| Blat-Cas9 | 831 | ITAKKTALVDISY | — |
| Blat-Cas9 | 900 | RIMENKTLV | — |
| Blat-Cas9 | 917 | VVYNSSIVR | — |
| Blat-Cas9 | 976 | SLYPNDLIF | — |
| Blat-Cas9 | 1009 | EVQEIHAYY | — |
| Blat-Cas9 | 1023 | STAAIEFIIHDGSYYA | — |
| Cas13d | 5 | KSFAKGMGV | — |
| Cas13d | 72 | FSHPKGYAVVANNPLY | — |
| Cas13d | 135 | YITNAAYAVNNISGL | — |
| Cas13d | 160 | STVYTYDEF | — |
| Cas13d | 267 | YISTLNYLY | — |
| Cas13d | 278 | ITNELTNSF | — |
| Cas13d | 357 | KVYTMMDFVIYRY | — |
| Cas13d | 397 | FVINLRGSF | — |
| Casl3d | 461 | VSAFSKLMYALTMFL | — |
| Cas13d | 495 | QSFLKVMPL | — |
| Cas13d | 528 | RLIKSFARM | — |
| Cas13d | 553 | RILGTNLSY | — |
| Cas13d | 565 | KALADTFSL | — |
| Cas13d | 596 | ISNKRFHYL | — |
| Cas13d | 702 | KIISLYLTVIY | — |
| Cas13d | 798 | KLYANYIKY | — |
| Cas13d | 864 | EVARYVHAY | — |
| Cas13d | 878 | EVNSYFQLYHYI | — |
| Cas13d | 905 | KVSEYFDAV | — |
| Cas13d | 925 | KLLCVPFGY | — |
| Cas-Phi | 79 | KASEAIQRYIYAL | — |
| Cas-Phi | 124 | HVQGLNLIFDHTLGR | — |
| Cas-Phi | 180 | ATNETGHLL | — |
| Cas-Phi | 192 | GINPSFYVY | — |
| Cas-Phi | 201 | QTISPQAYR | — |
| Cas-Phi | 223 | YVRDPNAPI | — |
| Cas-Phi | 269 | VTVPGLSPK | — |
| Cas-Phi | 330 | SLNALLDLF | — |
| Cas-Phi | 349 | NIVTFTYTLDAC | — |
| Cas-Phi | 361 | GTYARKWTL | — |
| Cas-Phi | 381 | LTATQTVAL | — |
| Cas-Phi | 396 | QTNPISAGI | — |
| Cas-Phi | 428 | LLKDISAYR | — |
| Cas-Phi | 492 | KMSSNTTFISEALL | — |
| Cas-Phi | 513 | QVFFTPAPK | — |
| Cas-Phi | 541 | RAYKPRLSV | — |
| Cas-Phi | 564 | RTSPEYLKL | — |
| Cas-Phi | 582 | RSINYVIEK | — |
| Cas-Phi | 595 | TQCQIVIPVIEDL | — |
| Cas-Phi | 647 | RTHRSFYVFEVRPER | — |
| CasX | 66 | MLLDDYTKMKEAILQV | — |
| CasX | 199 | VTKESTHPV | — |
| CasX | 233 | GTIASFLSKY | — |
| CasX | 284 | HTKEGVDAY | — |
| CasX | 300 | RMWVNLNLW | — |
| CasX | 361 | RVFWSGVTA | — |
| CasX | 374 | TILEGYNYL | — |
| CasX | 404 | RQFGDLLLY | — |
| CasX | 435 | KIAGLTSHI | — |
| CasX | 525 | IQYRNLLAW | — |
| CasX | 541 | REFYLLMNY | — |
| CasX | 578 | KAKVIDLTF | — |
| CasX | 608 | FIWNDLLSL | — |
| CasX | 759 | VTHDAVLVF | — |
| CasX | 780 | RTFMTERQY | — |
| CasX | 809 | YLSKTLAQYTSK | — |
| CasX | 863 | ITYYNRYKR | — |
| CasX | 939 | LNIARSWLF | — |
| CasX | 947 | FLNSNSTEF | — |
| CasX | 965 | FVGAWQAFY | — |
| Cj-Cas9 | 2 | RILAFDIGISSIGWAF | — |
| Cj-Cas9 | 63 | RLNHLKHLI | — |
| Cj-Cas9 | 209 | KQREFGFSFSK | — |
| Cj-Cas9 | 224 | EVLSVAFYK | — |
| Cj-Cas9 | 234 | ALKDFSHLVGNCSFF | — |
| Cj-Cas9 | 262 | FMFVALTRIINLLNNL | — |
| Cj-Cas9 | 325 | GTYFIEFKK | — |
| Cj-Cas9 | 398 | KALKLVTPL | — |
| Cj-Cas9 | 471 | KVHKINIEL | — |
| Cj-Cas9 | 552 | KMLEIDHIYPYSRSF | — |
| Cj-Cas9 | 569 | SYMNKVLVFT | — |
| Cj-Cas9 | 641 | YIARLVLNYTKDYLDFL | — |
| Cj-Cas9 | 685 | LTSALRHTWGF | — |
| Cj-Cas9 | 712 | IIAYANNSI | — |
| Cj-Cas9 | 720 | IVKAFSDFK | — |
| Cj-Cas9 | 849 | YTMDFALKV | — |
| Cj-Cas9 | 875 | ILMDENYEF | — |
| Cj-Cas9 | 911 | FTSSTVSLI | — |
| Cj-Cas9 | 949 | KSIGIQNLK | — |
| Cj-Cas9 | 962 | YIVSALGEV | — |
| Nme2-Cas9 | 0 | MAAFKPNPI | — |
| Nme2-Cas9 | 17 | GIASVGWAM | — |
| Nme2-Cas9 | 57 | LAMARRLAR | — |
| Nme2-Cas9 | 126 | WSAVLLHLIKHRGYL | — |
| Nme2-Cas9 | 207 | LQAELILLF | — |
| Nme2-Cas9 | 237 | LLMTQRPAL | — |
| Nme2-Cas9 | 271 | YTAERFIWL | — |
| Nme2-Cas9 | 347 | MEMKAYHAI | — |
| Nme2-Cas9 | 414 | ISFDKFVQI | — |
| Nme2-Cas9 | 425 | KALRRIVPL | — |
| Nme2-Cas9 | 583 | VEIDHALPFSRTW | — |
| Nme2-Cas9 | 671 | YVNRFLCQFVADHILLT | — |
| Nme2-Cas9 | 693 | RVFASNGQI | — |
| Nme2-Cas9 | 703 | NLLRGFWGL | — |
| Nme2-Cas9 | 728 | VVACSTVAMQQKITRFV | — |
| Nme2-Cas9 | 778 | FAQEVMIRV | — |
| Nme2-Cas9 | 819 | AVHEYVTPLFVSR | — |
| Nme2-Cas9 | 965 | NQYFIVPIYAWQVAENI | — |
| Nme2-Cas9 | 996 | YTFCFSLHK | — |
| Nme2-Cas9 | 1015 | KSKVEFAYY | — |
| Pp-Cas9 | 0 | MQNNPLNYI | — |
| Pp-Cas9 | 14 | GIASIGWAV | — |
| Pp-Cas9 | 186 | GSYTHTFSR | — |
| Pp-Cas9 | 195 | LDLLAEMELLF | — |
| Pp-Cas9 | 219 | TLLENLTALLMWQKPAL | — |
| Pp-Cas9 | 261 | YSAERFVWL | — |
| Pp-Cas9 | 304 | LTYAQVRAM | — |
| Pp-Cas9 | 313 | LALSDNAIF | — |
| Pp-Cas9 | 419 | KALHQILPL | — |
| Pp-Cas9 | 479 | KVINAVVRLYGSPARI | — |
| Pp-Cas9 | 546 | ILKMRLYEL | — |
| Pp-Cas9 | 631 | RVQTSGFSYAK | — |
| Pp-Cas9 | 665 | YVARFLCNFIADNMLLV | — |
| Pp-Cas9 | 697 | ALLRHRWGL | — |
| Pp-Cas9 | 722 | WACSTVAM | — |
| Pp-Cas9 | 829 | RLNEGLSVL | — |
| Pp-Cas9 | 878 | KAFAEPFYK | — |
| Pp-Cas9 | 931 | YFLVPIYTW | — |
| Pp-Cas9 | 965 | EMATFQFSL | — |
| Pp-Cas9 | 987 | TIFGYFNGL | — |
| Sauri-Cas9 | 78 | YQMIDLNNV | — |
| Sauri-Cas9 | 96 | RVKGLREPL | — |
| Sauri-Cas9 | 109 | FAIALLHIA | — |
| Sauri-Cas9 | 121 | GLHNISVSM | — |
| Sauri-Cas9 | 239 | KLMGRCTYF | — |
| Sauri-Cas9 | 258 | YSADLFNAL | — |
| Sauri-Cas9 | 403 | SLKCIHIVI | — |
| Sauri-Cas9 | 420 | NQMEIFTRL | — |
| Sauri-Cas9 | 467 | VINAVINRF | — |
| Sauri-Cas9 | 554 | LSNPTHYEV | — |
| Sauri-Cas9 | 564 | HIIPRSVSFDNSL | — |
| Sauri-Cas9 | 668 | KTYFSTHDY | — |
| Sauri-Cas9 | 713 | LVIANADFLFKTHKAL | — |
| Sauri-Cas9 | 839 | KTFEKLMTILNQY | — |
| Sauri-Cas9 | 914 | KSFRFDIYK | — |
| Sauri-Cas9 | 964 | DLFVGSFYY | — |
| Sauri-Cas9 | 975 | LIMYEDELF | — |
| Sauri-Cas9 | 1003 | ITYKDFCEV | — |
| Sauri-Cas9 | 1022 | KTIGKRVVL | — |
| Sauri-Cas9 | 1034 | YTTDILGNL | — |

To extend and complement the Cas9 screening efforts, the effect of the combinations of mutations on editing functionality, were recapitulated through in silico structural analyses using state-of-the-art protein structure prediction software: Google Deepmind's Alphafold. Through predicting the structures of high-confidence double mutants within the screen, we noticed a positive correlation between the position-specific local structure and confidence metric pLDDT, and the epistatic permissiveness of mutations within the screen (FIG. 13). This is to say that while pLDDT does not correlate with the effects of a mutation on editing functionality, it does correlate with how well combinations of mutations edit in comparison to the expected editing based on a null, additive model of epistasis. Mutation combinations with high epistatic scores, i.e. mutants with better editing than expected based on the effects of the individual mutations tend to have high pLDDT scores, while double mutants with negative epistasis rarely have high pLDDT scores. To validate this observation, the Alphafold workflow was used to assess this effect in third-party datasets across a variety of proteins (FIG. 13).

Using this pLDDT-epistasis connection, it is possible to subset de-immunizing mutations to exclude those which have a low likelihood of being epistatically permissive. This is of critical importance to moving further down the spectrum of de-immunization into combinations of mutations disrupting multiple epitopes, as would be needed to circumvent the immune response to Cas9 in a clinical context. Towards this end, single mutants were identified among several clinically-relevant Cas9 orthologs which are less likely to produce negative epistatic effects that will substantially reduce editing efficiency when combined with mutations across other epitopes (Table C). These mutations may contribute to de-immunized versions of these Cas9 orthologs upon further development.

TABLE C
High pLDDT mutations predicted to be
epistatically permissive: engineered de-immunized Cas9s would bear
combinations of these. pLDDT measures are standardized across
proteins. (Amino acid position is based upon methionine being
position “O”).

| Source protein | Mutation | pLDDT |
|---|---|---|
| Sp-Cas9 | S14T | 97.21972 |
| Sp-Cas9 | L34N | 96.29946 |
| Sp-Cas9 | A156G | 97.70565 |
| Sp-Cas9 | I161T | 96.70064 |
| Sp-Cas9 | L240C | 95.5989 |
| Sp-Cas9 | F374Q | 95.1961 |
| Sp-Cas9 | T444E | 96.71938 |
| Sp-Cas9 | L513D | 96.8859 |
| Sp-Cas9 | L615N | 95.7314 |
| Sp-Cas9 | F703T | 95.70707 |
| Sp-Cas9 | Y813E | 96.28403 |
| Sp-Cas9 | L815K | 95.2872 |
| Sp-Cas9 | K877N | 96.38619 |
| Sp-Cas9 | V1094T | 97.93581 |
| Sp-Cas9 | V1138F | 95.65631 |
| Sp-Cas9 | R1211G | 97.46792 |
| Sp-Cas9 | L1265D | 95.5318 |
| Sp-Cas9 | I1272N | 95.7981 |
| Sp-Cas9 | Y1293H | 95.3305 |
| Sa-Cas9 | I10E | 96.04671618970484 |
| Sa-Cas9 | L69G | 96.4474038803064 |
| Sa-Cas9 | L111G | 97.5246901174282 |
| Sa-Cas9 | Y238D | 95.7959234150505 |
| Sa-Cas9 | L261Q | 95.36556536646381 |
| Sa-Cas9 | I353R | 97.05582266121893 |
| Sa-Cas9 | H392V | 96.40816573690797 |
| Sa-Cas9 | N410Y | 97.39004196840122 |
| Sa-Cas9 | I457G | 96.99516367772516 |
| Sa-Cas9 | F564R | 96.04196929055283 |
| Sa-Cas9 | Y650G | 95.33378157800597 |
| Sa-Cas9 | F679H | 95.6997935920167 |
| Sa-Cas9 | I706E | 96.21671919911144 |
| Sa-Cas9 | P741Q | 96.76471819653675 |
| Sa-Cas9 | N803K | 95.70463233624467 |
| Sa-Cas9 | K923I | 95.81887787225756 |
| Sa-Cas9 | F924G | 96.3942586623205 |
| Sa-Cas9 | S964C | 97.46403 / 08042462 |
| Sa-Cas9 | N974F | 96.28983910214497 |
| Sa-Cas9 | I1016Y | 96.88397168045735 |
| Cj-Cas9 | A5C | 95.42456861125262 |
| Cj-Cas9 | L70F | 95.96944171410574 |
| Cj-Cas9 | Q210P | 95.53016253543593 |
| Cj-Cas9 | L235T | 95.59476304574478 |
| Cj-Cas9 | R269D | 95.57444080957478 |
| Cj-Cas9 | E330N | 95.4689925 8457661 |
| Cj-Cas9 | L400H | 97.57050152845376 |
| Cj-Cas9 | E478V | 95.969187864883 |
| Cj-Cas9 | E555T | 97.12260803185546 |
| Cj-Cas9 | V576W | 96.5828278683 5 804 |
| Cj-Cas9 | A643L | 98.33207173472285 |
| Cj-Cas9 | R690V | 95.74871521269799 |
| Cj-Cas9 | S719N | 97.95496295071534 |
| Cj-Cas9 | F724S | 96.22792344876726 |
| Cj-Cas9 | T850N | 95.41876846442275 |
| Cj-Cas9 | Y881H | 96.86255346228734 |
| Cj-Cas9 | S917Q | 96.2179175861716 |
| Cj-Cas9 | Q954D | 97.52472596837454 |
| Cj-Cas9 | V970H | 97.50358598810789 |
| Cas-Phi | Q85Y | 96.81295319454516 |
| Cas-Phi | V125L | 95.12108676130539 |
| Cas-Phi | T184R | 95.1772971119881 |
| Cas-Phi | G192K | 96.31102265697412 |
| Cas-Phi | R209V | 95.17061366434854 |
| Cas-Phi | D226G | 96.73279555432283 |
| Cas-Phi | F338S | 96.4409845122545 |
| Cas-Phi | T356A | 98.46569344377559 |
| Cas-Phi | A364Q | 96.9761260500557 |
| Cas-Phi | A388I | 96.4379215589604 |
| Cas-Phi | S401V | 95.34453659826832 |
| Cas-Phi | R436L | 96.35999339995325 |
| Cas-Phi | N496L | 95.26296308688346 |
| Cas-Phi | P518L | 95.59765365564213 |
| Cas-Phi | L547S | 95.62780934854082 |
| Cas-Phi | T565D | 96.87703607854039 |
| Cas-Phi | K590V | 96.03570468291413 |
| Cas-Phi | F655E | 95.63672115804113 |
| Cas-X | M66V | 96.66213349474216 |
| Cas-X | E202Q | 96.24421703611794 |
| Cas-X | T234G | 95.41585619353467 |
| Cas-X | K286F | 97.0668726663819 |
| Cas-X | R300D | 97.2909813029547 |
| Cas-X | V362F | 96.10786479141233 |
| Cas-X | L411H | 96.34639451882234 |
| Cas-X | A437P | 96.2646137070106 |
| Cas-X | Q526K | 96.16734163961726 |
| Cas-X | E542R | 95.86976977015941 |
| Cas-X | S615Q | 95.14185165348067 |
| Cas-X | L765C | 95.79820047448582 |
| Cas-X | R786P | 96.47451095139678 |
| Cas-X | Y817I | 97.2015805474868 |
| Cas-X | W945N | 97.64293477689652 |
| Cas-X | F955T | 95.70627543561199 |
| Cas-X | G967K | 97.79349327320138 |
| AsCas12a (Cpf1) | N8W | 95.92802445100253 |
| AsCas12a (Cpf1) | L210K | 95.10630901001409 |
| AsCas12a (Cpf1) | S235Y | 96.11142315459921 |
| AsCas12a (Cpf1) | I363T | 95.60480914360495 |
| AsCas12a (Cpf1) | G475N | 95.86498924015017 |
| AsCas12a (Cpf1) | F556I | 96.03740222311919 |
| AsCas12a (Cpf1) | R680Y | 95.47851908471684 |
| AsCas12a (Cpf1) | K795H | 96.45630801435865 |
| AsCas12a (Cpf1) | Q936R | 95.82214160865102 |
| AsCas12a (Cpf1) | I978T | 96.48655903840778 |
| AsCas12a (Cpf1) | V988A | 96.70259243879435 |
| AsCas12a (Cpf1) | C1024A | 97.018068389001 |
| AsCas12a (Cpf1) | Y1042D | 96.87401194635731 |
| AsCas12a (Cpf1) | V1215T | 95.68310287241839 |
| AsCas12a (Cpf1) | N1294S | 96.90764727328744 |

Inserting T-regitopes. A further de-immunization approach beyond the mutation or deletion of MHC-binding cores is to inhibit immune activation at the level of cell signaling. Paramount in facilitating this process is the induction of inhibitory T-reg cells which modulate the activity of other T-cells to promote tolerance of foreign antigens. One canonical pathway in which T-reg induction helps to facilitate tolerance of antigenic diversity is in the potential adaptive response to antibodies themselves. As antibodies are highly polymorphic and undergo substantial mutational remodeling during the process of B-cell activation and maturation. As a result, it might be expected that these neo-antigens may create an adaptive immune response. However, this potential problem is mitigated by certain sequences within the conserved regions of immunoglobulins, termed T-regitopes, which are recognized specifically by regulatory T-cells to promote a tolerogenic response to proteins bearing these T-regitopes. Correspondingly these immune-modulating sequences can be utilized in the context of foreign protein therapeutics to dampen or avoid a problematic immune response.

Additionally, a multifaceted approach was used to de-immunizing the AAV capsid itself. Applying similar methodological principles as above, an alignment of the major AAV serotypes was constructed, identifying 21 conserved regions across serotypes which may constitute cross-reacting T-cell epitopes. By definition, these regions will be the most conserved across AAV serotypes, and presumably the least mutation tolerant. This presents a unique challenge to engineering efforts and predicts that the vast majority of mutants in these regions will be strongly deleterious.

To narrow down the space of possible mutations, and to increase the probability of including functional mutations in the library, a large alignment of over 200 AAV variants was also constructed which were sequenced from natural or engineered sources. This larger alignment contains many SNPs which may constitute some of the natural variation among AAVs, some of which occur in the highly conserved regions as identified. As these mutations have been observed to occur in a natural and presumably functional context, they are much more likely to serve as useful library members (Table D)

TABLE D
Immunogenic and conserved AAV-VP1 epitopes and de-immunizing mutations
(methionine is position ″0″)

| Source | AA position | | |
|---|---|---|---|
| protein | (0-based) | Epitope | Mutation |
| AAV5-VP1 | 41 | ARGLVLPGY (SEQ ID NO: 4 from | P47L; R42G; Y49C |
| | | 41-49) | |
| AAV5-VP1 | 51 | YLGPGNGLD (SEQ ID NO: 4 from | G55S; D59Y; G57S; N56H |
| | | 51-59) | |
| AAV5-VP1 | 84 | AGDNPYLKYNHADAEFQE (SEQ | L90P; Y89H; E98K; A95V; |
| | | ID NO: 4 from 84-101) | D96G; F99L |
| AAV5-VP1 | 106 | DTSFGGNLG (SEQ ID NO: 4 from | T107A; S108P |
| | | 106-114) | |
| AAV5-VP1 | 116 | AVFQAKKRVLEP (SEQ ID NO: 14 | Q119R; V124A; R123L |
| | | from 116-127) | |
| AAV5-VP1 | 129 | GLVEEGAKTAPTGK (SEQ ID | E133Q; G134V; T137A; |
| | | NO: 14 from 129-142) | V131A; E132G |
| AAV5-VP1 | 206 | QGADGVGNASGDWHCDSTWMG | A214V; T223A; S222T |
| | | DRV (SEQ ID NO: 14 from 206-229) | |
| AAV5-VP1 | 263 | YFGYSTPWGYFDFNRFH (SEQ ID | S267A; Y272H; F273L |
| | | NO: 14 from 263-279) | |
| AAV5-VP1 | 283 | SPRDWQRLINN (SEQ ID NO: 14 | W287R; I291V; L290P |
| | | from 283-293) | |
| AAV5-VP1 | 303 | RVKIFNIQVKEVT (SEQ ID NO: 14 | K312E; I309V |
| | | from 303-315) | |
| AAV5-VP1 | 320 | TTTIANNLTSTVQVFTD (SEQ ID | T328A |
| | | NO: 14 from 320-336) | |
| AAV5-VP1 | 395 | MLRTGNNFEF (SEQ ID NO: 14 | N400D; F402L |
| | | from 395-404) | |
| AAV5-VP1 | 408 | FEEVPFHSS (SEQ ID NO: 50 from | S416M; F413L; S415T |
| | | 408-416) | |
| AAV5-VP1 | 599 | RDVYLQGPIWAKIP (SEQ ID | Q604R; I607T; P606Q |
| | | NO: 50 from 599-612) | |
| AAV5-VP1 | 624 | MGGFGLKHPPP (SEQ ID NO: 50 | L629P; K630E; F627L; |
| | | from 624-634) | H631R |
| AAV5-VP1 | 657 | SFITQYSTGQV (SEQ ID NO: 50 | T664A; S663G |
| | | from 657-667) | |
| AAV5-VP1 | 677 | KENSKRWNPE (SEQ ID NO: 50 | N684D; R682C; W683R |
| | | from 677-686) | |
| AAV5-VP1 | 713 | RPIGTRYLTR (SEQ ID NO: 50 from | T717S; Y719F; L720P |
| | | 713-722) | |

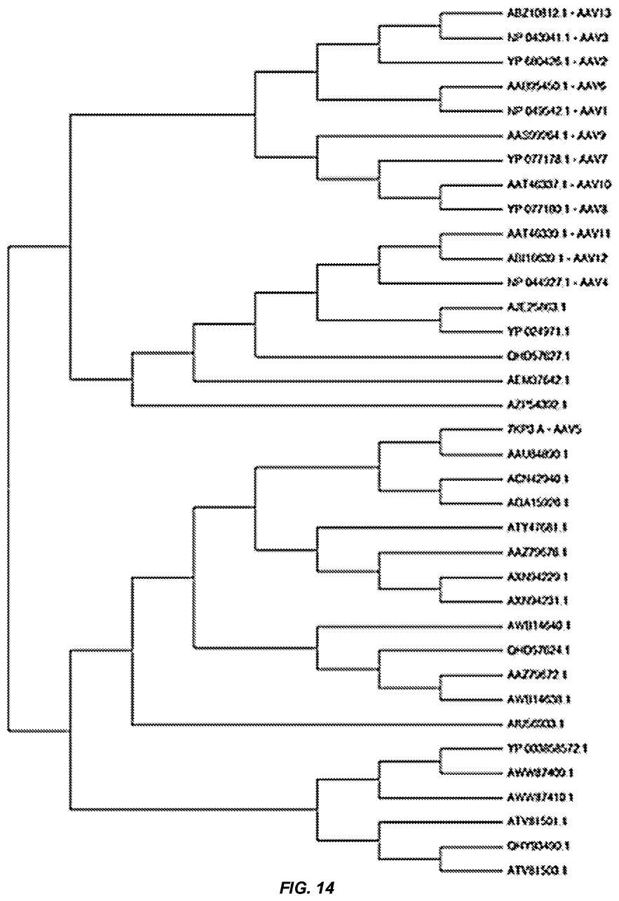

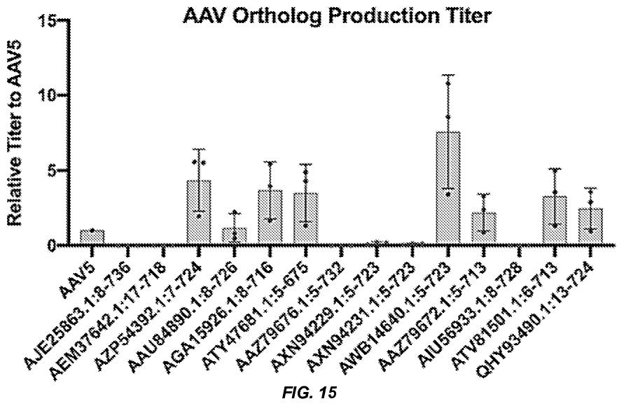

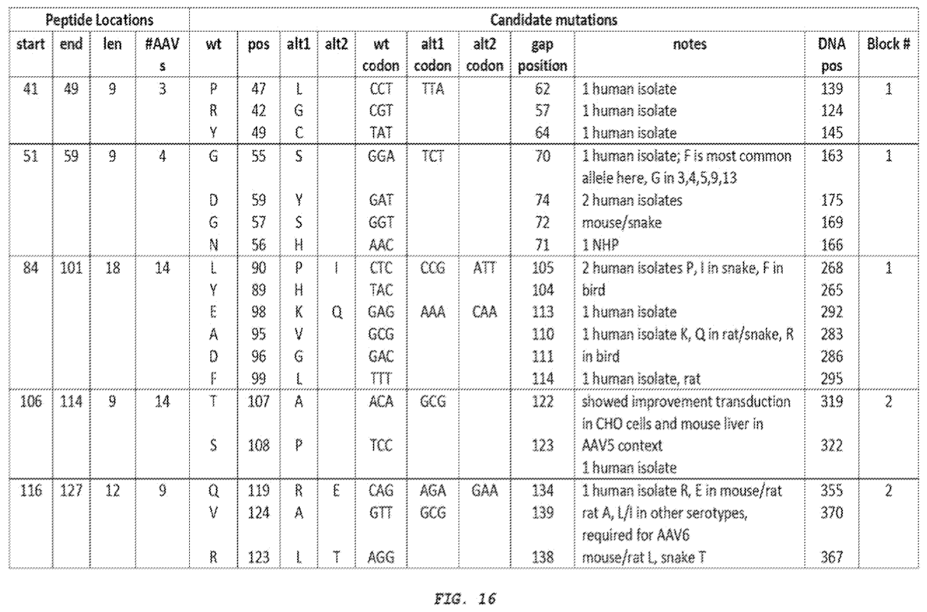

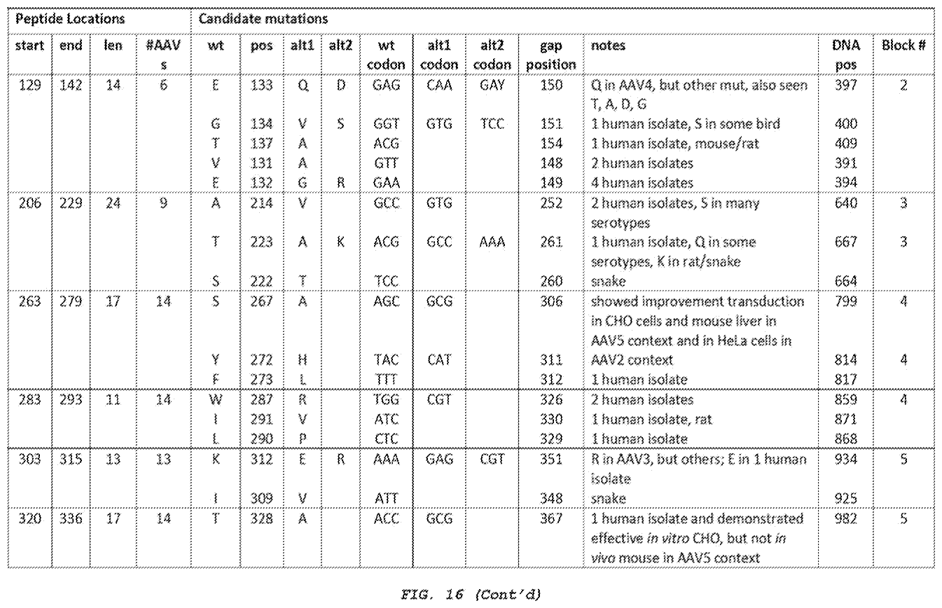

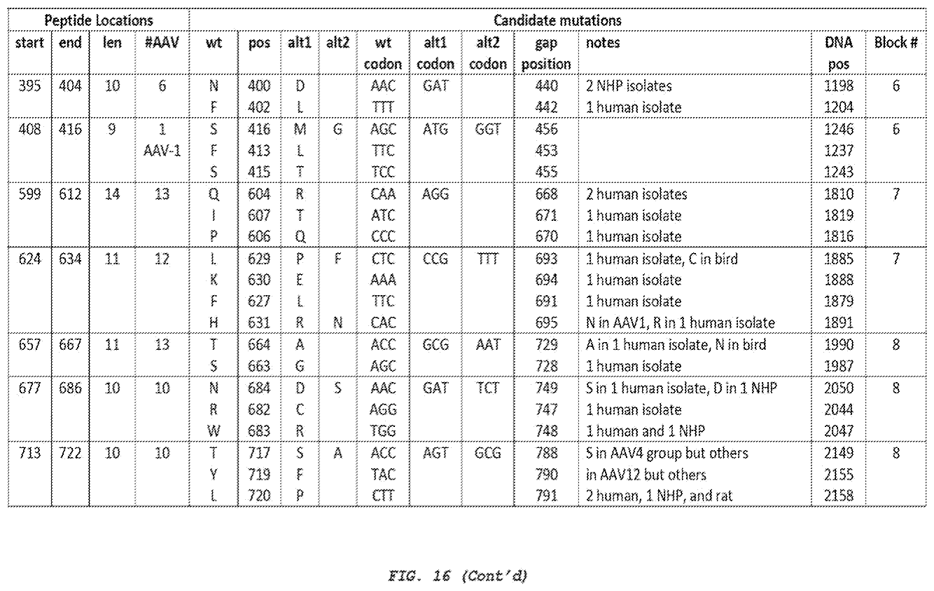

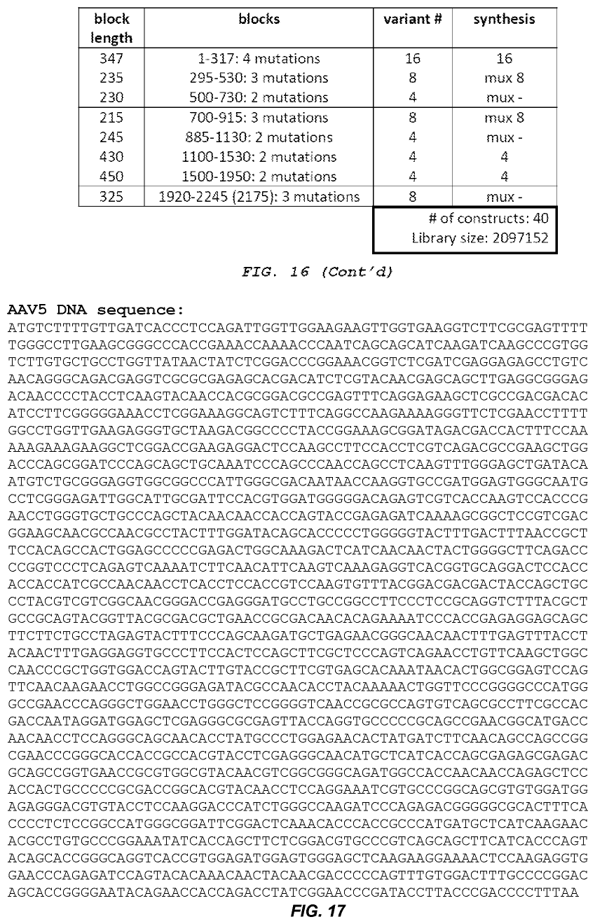

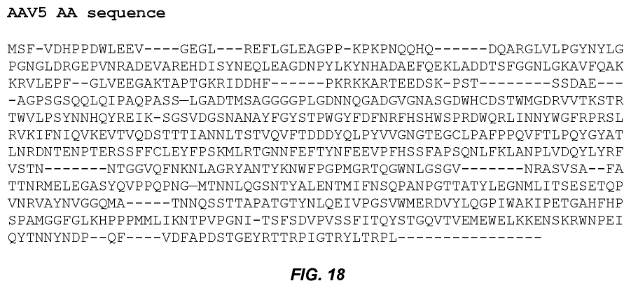

Given the substantially more challenging prospect of engineering multiple mutations simultaneously in a highly conserved structural protein such as a viral capsid, an orthogonal approach was taken to identifying a potentially immune orthogonal AAV capsid by exploring the utility of highly divergent natural orthologs from various mammalian species which have not yet been thoroughly tested for use as in vivo vectors. On this front, 687 AAV capsid sequences were scraped from publicly available sequencing data, filtering this set down to 224 capsids by removing truncated and redundant sequences. From there the list of potential AAVs was narrowed by removing sequences within the main clade of human AAVs that are unlikely to boast novel structural properties (104), removing sequences from non-mammalian hosts to maximize the potential of identifying an AAV with strong transduction potential in the human setting (31), and finally condensing similar sequences to a final set of 23 natural AAV orthologs which we are in the process of constructing and testing for viral formation and transduction efficiency (FIG. 14).

The capsid sequences of each ortholog were chemically synthesized and split into two blocks from the 5′ and 3′ end. In order to assemble the two blocks from each ortholog, a process similar to the one used for assembling the Cas9 variants was done. The first step involves the annealing-extension step followed by the addition of primers to amplify the full length ortholog. Then the full length capsid sequences (see, e.g., SEQ ID NO:2-48, which include both polynucleotide coding sequences and polypeptide sequence) are then inserted and cloned into a pAAV RC2 vector via Gibson assembly.

To test for viral formation, a triple transfection method was used for AAV production using HEK 293T cells and purified with an iodixanol gradient. Cells were transfected once the confluence reached between 70% and 90% in a 5×15 cm² plate. Plates were transfected with 10 μg of the plasmid with full length inserted ortholog capsid sequence or pxR-5 used as a control, 10 μg of transfer vector, and 10 μg of pAd4 helper vector. The virus was collected after 72 hours and purified using an iodixanol-density-gradient ultracentrifugation method. After dialysis and filtration, the virus was quantified by qPCR. Results of viral formation were analyzed by comparing titers of the AAV orthologs to AAV5 titers used as a control. The viral formation of 14 AAV orthologs were tested and have demonstrated 8 AAV orthologs successfully packaging and producing virus (FIG. 15).

It will be understood that various modifications may be made without departing from the spirit and scope of this disclosure. Accordingly, other embodiments are within the scope of the following claims.

What is claimed is:

1. A method for engineering a protein or virus to be less immunoreactive or to be immuno-silent, comprising:
identifying target regions of the polynucleotide sequence encoding a protein or virus that are predicted to have human leukocyte antigen (HLA)-binding and/or peptide immunogenicity;
identifying single nucleotide polymorphisms (SNPs) or mutations in the targeted region and other regions that are not deleterious to the functioning of the protein to obtain mutational criteria;
screening a library assembled using standard synthesis and assembly methods by applying the above identifying criteria to find one or more functional variants of the protein or virus;
sequencing the one or more functional variants of the protein or virus;
mapping genotype to phenotype from the sequences of the functional variants to identify variant candidates that are likely functionally active and have mutations that result in the protein or virus exhibiting less immunogenicity or are immune-silent.

2. The method of claim 1, wherein the protein is a CRISPR associated protein.

3. The method of claim 1, wherein the CRISPR associated protein is a Cas9.

4. The method of claim 3, wherein the Cas9 is Streptococcus pyogenes Cas9 (SpCas9).

5. The method of claim 1, wherein the target regions are identified using a model that predicts human leukocyte antigen (HLA)-binding and peptide immunogenicity.

6. The method of claim 5, wherein the prediction model is selected from NetMHC, MHCAttnNet, MHCSeqNET, ACME, NetMHCpan EL 4.1, NetMHCstabpan, SMM, SMMPMBEC, PickPocket, Comblib_Sidney2008, NetMHCcons, MHCflurry 2.0, and IConMHC.

7. The method of claim 4, wherein the SNPs or mutations are identified by using phylogenetic methods to scan natural variation among naturally occurring SpCas9, mutations generated in the course of research and engineering efforts, and the Cas9 orthologs of closely related bacterial species.

8. The method of claim 4, wherein the SNPS or mutations are identified, or further identified, by using immunological prediction of candidate mutations to ensure significant loss of immunogenicity within the targeted region in order to preserve function while reducing immunogenicity.

9. The method of claim 1, wherein the virus is an adeno-associated virus (AAV).

10. The method of claim 9, wherein the AAV is selected from AAV1, AAV2, AAV5, AAV6, AAV7, and AAV8.

11. The method of claim 10, wherein the AAV is AAV5.

12. The method of claim 9, wherein the target regions are identified by aligning conserved sequence regions across AAV serotypes.

13. The method of claim 12, wherein the SNPs or mutations are identified by aligning the sequences of 50, 60, 70, 80, 90, 100, 110, 120, 130, 140, 150, 160, 170, 180, 190, 200 or more AAV variants that have been sequenced from natural or engineered sources.

14. The method of claim 13, wherein the SNPs or mutations are located in the target regions.

15. The method of claim 1, wherein long-read sequencing technologies capable of sequencing the entire sequence of each protein or viral variant in one sequencing reaction is used to sequence the functional variants of the protein or virus.

16. The method of claim 15, wherein the long-read sequencing technologies is capable of generating 10-15 Gb of sequencing reads per run.

17. The method of claim 1, wherein the method further comprises:
evaluating the immunoreactivity of variant candidates in one or more immunoassays.

18. The method of claim 17, wherein the one or more immunoassays comprise detecting the presence of antibodies to the variant candidates (AVA antibodies), when the variant candidates are administered in vivo to an animal.

19. The method of claim 17, wherein the one or more immunoassays comprise an enzyme-linked immunosorbent assays (ELISAs), electrochemiluminescence (ECL) assays and/or antigen-binding tests, wherein the one or more immunoassays utilize AVA antibodies.

20. An isolated polynucleotide having at least 80%, 90%, 95%, 97%, 98%, or 99% sequence identity to SEQ ID NO:1, and encoding a polypeptide of SEQ ID NO:2 having one or more mutations selected from the group consisting of: P28L, L237C, Y286Q, S318(H/C), S368C, F498T, L514(T/G), L616G, L623Q, L636D, F704A, L727(P/G), L816D, Y1016(K/G), L1245G, I1273Q, L1282(A/E), and/or Y1294Q and wherein the polypeptide has Cas9 like activity.

21. An isolated polypeptide having a sequence that has at least 90%, 95%, 97%, 98%, or 99% sequence identity to the sequence presented in SEQ ID NO:2, wherein the protein has Cas9 like activity and is immuno-silenced.

22. The isolated polypeptide of claim 21, wherein the protein has less immunogenicity than wild-type Cas9 of SEQ ID NO:2.

23. The protein of claim 21, wherein the polypeptide comprises the sequence of SEQ ID NO:2 and having one or more mutations selected from the group consisting of: P28L, L237C, Y286Q, S318(H/C), S368C, F498T, L514(T/G), L616G, L623Q, L636D, F704A, L727(P/G), L816D, Y1016(K/G), L1245G, I1273Q, L1282(A/E), and/or Y1294Q.

24. A CRISPR-Cas9 system comprising the protein of claim 20.

25. The CRISPR-Cas9 system of claim 24, wherein the CRISPR-Cas9 system further comprises RNA that comprise a shot sequence that binds to a specific target sequence of DNA in a genome.

26. A virus encoded by a polynucleotide sequence having at least 90%, 95%, 97%, 98%, or 99% sequence identity to the sequence of SEQ ID NO:49, wherein the virus has AAV5 like activity and is immuno-silenced.

27. A AAV viral capsid having at least 90%, 95%, 97%, 98%, or 99% sequence identity to the sequence presented in SEQ ID NO:50, wherein the virus has AAV5 like activity and is immuno-silenced.

28. The AAV capsid of claim 27, wherein the viral capsid comprises a sequence of SEQ ID NO:50 and comprises two or more mutation selected from the group consisting of R42G, P47L, Y49C, G55S, N56H, G57S, D59Y, Y89H, L90(P/I), A95V, D96G, E98(K/Q), F99L, T107A, S108P, Q119(R/E), R123(L/T), V124A, V131A, E132(G/R), E133(Q/D), G134(V/S), T137A, A214V, S222T, T223(A/K), S267A, Y272H, F273L, W287R, L290P, I291V, I309V, K312(E/R), N400D, F402L, F413L, S415T, S416(M/G), Q604R, P606Q, I607T, F627L, L629(P/F), K630E, H631(R/N), S663G, T664A, R682C, W683R, N684(D/S), T717(S/A), Y719F, and L720P.

29. The virus of claim 26, wherein the virus has less immunogenicity than a wild-type AAV5.

30. An AAV system comprising the virus of claim 26.

31. The AAV system of claim 30, wherein the AAV system is used for gene therapy.
